(19) United States
(12) Patent Application Publication
(10) Pub. No.: US 2026/0027227 A1
(43) Pub. Date: Jan. 29, 2026

(54) NOVEL THIOL REDUCTANT, METHOD AND USE THEREOF

(71) Applicant: SUZHOU BIOREINNO BIOTECHNOLOGY LIMITED COMPANY, Suzhou, Jiangsu (CN)

(72) Inventors: Lili WU, Suzhou, Jiangsu (CN); Ao JI, Suzhou, Jiangsu (CN); Yajun RAN, Suzhou, Jiangsu (CN); Yaru SHAO, Suzhou, Jiangsu (CN); Yicheng WANG, Suzhou, Jiangsu (CN)

(21) Appl. No.: 18/995,114

(22) PCT Filed: Aug. 22, 2023
(86) PCT No.: PCT/CN2023/114310

§ 371 Date: Jan. 15, 2025

(30) Foreign Application Priority Data
Aug. 22, 2022 (WO) PCT/CN2022/113992
Sep. 20, 2022 (WO) PCT/CN2022/119955
Sep. 20, 2022 (WO) PCT/CN2022/119999
Jan. 19, 2023 (WO) PCT/CN2023/073070

(51) Int. Cl. A61K 47/68; C07F 9/50; C07F 9/58; C07F 9/59; C07F 9/60; C07F 9/6506; C07F 9/6509; C07F 9/6533; C07F 9/655; C07K 16/32

(52) U.S. Cl. CPC A61K 47/6851; A61K 47/6803; A61K 47/68031; C07F 9/5004; C07F 9/5018; C07F 9/5022; C07F 9/5054; C07F 9/58; C07F 9/59; C07F 9/60; C07F 9/6506; C07F 9/650905; C07F 9/650952; C07F 9/6533; C07F 9/6552; C07K 16/32; C07K 2317/24

(57) ABSTRACT

The present disclosure relates to a novel thiol reductant having the formula (I), the preparation and the use in the preparation of an antibody with thiol group site-specific modifications with improved homogeneity.

### CROSS-REFERENCE TO RELATED APPLICATIONS

This application claims the priority to PCT Application No. PCT/CN2022/113992, filed on Aug. 22, 2022, PCT Application No. PCT/CN2022/119999, filed on Sep. 20, 2022, PCT Application No. PCT/CN2022/119955, filed on Sep. 20, 2022, and PCT Application No. PCT/CN2023/073070, filed on Jan. 19, 2023. The contents of the prior PCT applications are considered as a part of the present disclosure and are incorporated herein in its entirety.

### TECHNICAL FIELD

The disclosure relates to a novel thiol reductant, method and use thereof. The thiol reductant could be used in antibody modification.

### BACKGROUND

The statements in this section merely provide background information related to the present disclosure and do not necessarily constitute prior art.

Antibody-drug conjugates (ADCs) are innovative biopharmaceutical products in which a monoclonal antibody is linked to a small molecule drug with a stable linker. ADCs ideally combine the specificity of antibodies and high potency of cytotoxic drugs by delivering potent cytotoxic drugs to antigen-expressing cells, thereby enhancing their targeted cytotoxic activity.

Generally, antibody conjugation to cytotoxic agents commonly involves conjugation to exposed residues including lysines or reduction of disulfide bonds to expose free interchain cysteines on a therapeutic IgG (Immunoglobulin G) antibody. There are other, more recent approaches that introduce conjugation sites to the mAb such as site-specific glycan conjugation, cysteine engineering, incorporation of unnatural amino acids and coupling short peptide tags to drug-linkers. There are typically 80 lysine residues on an antibody; however, less than ten residues are chemically accessible for conjugation. Cysteine conjugation eventuates in the reduction of four interchain disulfide bonds. These bonds are reduced under specific conditions and subsequently result in two, four, six or eight exposed sulfhydryl groups. Both Cys and Lys conjugation methods result in heterogeneous mixtures. (“Advances and Limitations of Antibody Drug Conjugates for Cancer”. Biomedicines. 2021 August; 9 (8): 872.)

The drug-antibody ratio (DAR), or number of drug molecules conjugated to a single ADC, is very important for the determination of efficacy of ADCs. DAR widely varies and depends on other ADC variables. The DAR values are also dependent on the site of conjugation and the use of light or heavy conjugated chains. The DAR value influences the effectiveness of the medicine due to the depression in potency caused by low drug loading, while elevated drug loading can impact toxicity and pharmacokinetics (“Introduction to Antibody-Drug Conjugates”. Antibodies (Basel). 2021 December; 10 (4): 42). The conventional non-specific conjugation and conjugate distribution are largely influenced by factors such as pH, concentration, salt concentration, and co-solvents, so establishing a robust conjugation process always is challenging.

A number of methods have been developed to improve the homogeneity of ADCs. For example, Genentech's THIOMAB technology is developed based on improve the homogeneity of ADCs through antibody engineering, by introducing cysteine in the primary sequence of the antibody and realizing site-directed coupling to improve the uniformity of the product (“Cysteine-Based Coupling: Challenges and Solutions”. Bioconjug Chem. 2021 Aug. 18; 32 (8): 1525-1534.)

US20210040145 discloses a 14-amino acid peptide Tub-tagf used to the C-terminus of any POI and catalyzes the addition of a variety of different tyrosine derivatives. Taking advantage of this enzyme, Tub-tag technology repurposed tubulin-tyrosine ligase for the attachment of functional moieties at the C-terminus of antibody to homogeneously generate antibody conjugates with DAR 2.

WO2018036438 discloses a method to generate an ADC by using a technology named K-Lock, which can selectively react a well design linker-drug with four specific lysine residues on Fab of an IgG antibody, and yield ADC product comprising D2 (DAR value about 2) up to 50%. Finally, pure D2 can be achieved from further purification.

However, those technologies involve protein engineering and/or enzyme catalysis, so that those technologies suffer from several drawbacks, such as lower level of antibody expression, immunogenicity risk, complicated purification, and/or high cost.

Therefore, antibody-drug conjugates with improved homogeneity could provide benefits in terms of better stability and lower immunogenicity, and further result in therapeutic benefits, for example, better efficacy and lower toxicity. So, novel reductant and processes for preparing ADCs with high homogeneity are highly desirable and long-term pursuit.

### SUMMARY

For the above-mentioned purpose, provided herein is a compound having the following formula (I):

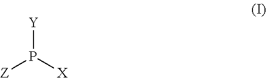

or a salt, solvate, stereoisomer thereof, which characterized in that,
X, Y and Z independently covalently connect the phosphorus atom through P—C bond, which is P—C(sp³) or P—C(sp²);
X is of formula (II)

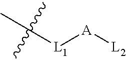

L₁ is selected from the group consisting of —CH(R₁)—, —C(CH₃)(R₁)—, —CH(R₁) CH(R₂)—, —CH(R₁)CH(R₂)CH(R₃)—, aryl group which is optionally independently substituted with group containing at least a coordinating atom selected from N, O and S, and heteroaryl group which is optionally independently substituted with group containing at least a coordinating atom selected from O and S;
R₁, R₂ and R₃ independently are H, C₁-C₅ alkyl group, C₁-C₅ hydroxyalkyl group, C₁-C₅ carboxy alkyl group, C₁-C₅ hydroxylamine alkyl group, C₁-C₅ N-hydroxy amide alkyl group, aryl group or heteroaryl group; or
R₂ or R₃ forms a 5-6 membered optionally substituted ring with L₂;
A is optionally present and is —C(O)—, or —C(O)J-;
J is organic group comprising amino or imino group and carbonyl group at the same time, of which the amino or imino group forms amide group with —C(O), the carboxyl group optionally covalently links to L₂;
L₂ is optionally present, L₂ works as transition metal chelator motif and is —N(R₄)(R₅) or hydroxy;
R₄ and R₅ independently are hydrogen, C₀-C₅ hydroxyalkyl group, C₁-C₅ alkyl group, C₁-C₅ alkoxy group, —(CH₂)n¹(OCH₂CH₂O)n²CH(R₆)CO(R₇), optionally substituted 5-6 membered saturated heterocyclic group, optionally substituted arylalkyl group, optionally substituted aryl alkoxy group, optionally substituted aryl group, optionally substituted heteroaryl group, optionally substituted heteroaryl alkyl group, or R₄ and R₅ form a 5-6 membered optionally substituted ring, R₄ or R₅ forms a 5-6 membered optionally substituted ring with R₂ or R₃;
R₆ is hydrogen, amino, C₁-C₅ alkyl, C₁-C₅ hydroxyalkyl group, C₁-C₅ carboxy alkyl group, aryl group, optionally substituted arylalkyl group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4;
R₄ and R₅ are not hydroxy at the same time;
Y is same as X,
Z is same as X, or
Y and Z independently are 5-6 membered optionally substituted saturated heterocyclic group, C₁-C₅ alkyl group, C₁-C₅ hydroxyalkyl group, aryl group, C₁-C₅ carboxy alkyl group, 5-6 membered optionally substituted cycloalkyl group, or

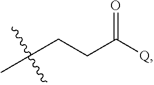

C(O)Q is ester group, imide group or amide group,
X, Y and Z are not —CH₂CH₂C(O)OH at the same time.

In one aspect, provided herein is a composition comprising the compound described above and transition metal ions.

In one aspect, provided herein is a method of preparing the compound described above.

In one aspect, provided herein is the use of the compound described above or the composition described above in an antibody modification.

In some embodiments, the antibody is modified by selectively reducing the interchain S-Sbonds of an antibody, optionally, the antibody is modification by selective reducing one of the interchain S—S bond.

In some embodiments, provided herein is the use of the compound described above or the composition described above in the preparation of an antibody with thiol group site-specific modifications, optionally, the antibody with thiol group site-specific modifications is an antibody drug conjugate (ADC).

In one aspect, provided herein is a method of preparing the antibody with thiol group site-specific modifications, which characterized in that, the thiol group (s) is/are reduced from the interchain disulfide bonds within the antibody, and the method comprises using the compound described above and the transition metal ions or using the composition described above.

In one aspect, provided herein is the antibody with thiol group site-specific modifications prepared by the methods described above.

In one aspect, provided herein is use of the antibody with thiol group site-specific modifications prepared by the methods described above in the manufacture of a therapeutic agent for preventing, diagnosing or treating a disease.

In one aspect, provided herein is a pharmaceutical composition comprising the antibody with thiol group site-specific modifications prepared by the methods described above and at least a pharmaceutically acceptable carrier.

In one aspect, provided herein is a method of preventing, diagnosing or treating a disease in a subject in need thereof, comprising administrating to the subject a therapeutically effective amount of the antibody with thiol group site-specific modifications provided above, or the pharmaceutical composition provided above.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 shows HIC-HPLC chromatogram of Trastuzumab-[MC-VC-PAB-MMAE]₂ conjugate prepared of Example 34. HIC-HPLC is short for Hydrophobic interaction chromatography-High performance liquid chromatography.

FIG. 2 shows HIC-HPLC chromatogram of Sacituzumab-[MC-VC-PAB-MMAE]₂ conjugate prepared of Example 35.

FIG. 3 shows HIC-HPLC chromatogram of Belantamab-[MC-VC-PAB-MMAE]₂ conjugate prepared of Example 36.

FIG. 4 shows HIC-HPLC chromatogram of Trastuzumab-[MC-VC-PAB-MMAE]₂ conjugate prepared of Comparative Example 1.

FIG. 5 shows HIC-HPLC chromatogram of Sacituzumab-[MC-VC-PAB-MMAE]₂ conjugate prepared of Comparative Example 2.

FIG. 6 shows HIC-HPLC chromatogram of Belantamab-[MC-VC-PAB-MMAE]₂ conjugate prepared of Comparative Example 3.

FIG. 7 shows HIC-HPLC chromatogram of Trastuzumab-[MC-VC-PAB-MMAE]₂ conjugate prepared of Example 37.

FIG. 8 shows HIC-HPLC chromatogram of Sacituzumab-[MC-VC-PAB-MMAE]₂ conjugate prepared of Example 38.

FIG. 9 shows HIC-HPLC chromatogram of Belantamab-[MC-VC-PAB-MMAE]₂ conjugate prepared of Example 39.

FIG. 10 shows HIC-HPLC chromatogram of Trastuzumab-[MC-VC-PAB-MMAE]₂ conjugate prepared of Comparative Example 4.

FIG. 11 shows HIC-HPLC chromatogram of Sacituzumab-[MC-VC-PAB-MMAE]₂ conjugate prepared of Comparative Example 5.

FIG. 12 shows HIC-HPLC chromatogram of Belantamab-[MC-VC-PAB-MMAE]₂ conjugate prepared of Comparative Example 6.

FIG. 13 shows HIC-HPLC chromatogram of Trastuzumab-[MC-VC-PAB-MMAE]₂ conjugate prepared of Example 40.

FIG. 14 shows HIC-HPLC chromatogram of Sacituzumab-[MC-VC-PAB-MMAE]₂ conjugate prepared of Example 41.

FIG. 15 shows HIC-HPLC chromatogram of Belantamab-[MC-VC-PAB-MMAE]₂ conjugate prepared of Example 42.

FIG. 16 shows HIC-HPLC chromatogram of Sacituzumab-[MC-VC-PAB-MMAE]₂ conjugate prepared of Comparative Example 8.

FIG. 17 shows HIC-HPLC chromatogram of Belantamab-[MC-VC-PAB-MMAE]2 conjugate prepared of Comparative Example 9.

FIG. 18A-H show HIC-HPLC chromatogram of Trastuzumab-[MC-VC-PAB-MMAE]₂ conjugate prepared of Examples 43-50, wherein, the reductant is TCEP-1, TCEP-2, TCEP-3, TCEP-4, TCEP-5, TCEP-6, TCEP-7 and TCEP-8.

FIG. 19A-H show HIC-HPLC chromatogram of Trastuzumab-[MC-VC-PAB-MMAE]₂ conjugate prepared of Examples 51-58, wherein, the reductant is TCEP-9, TCEP-10, TCEP-15, TCEP-18, TCEP-19, TCEP-20, TCEP-21 and TCEP-23.

FIG. 20A-G show HIC-HPLC chromatogram of Trastuzumab-[MC-VC-PAB-MMAE]₂ conjugate prepared of Examples 59-65, wherein, the reductant is TCEP-24, TCEP-25, TCEP-26, TCEP-28, TCEPA, TCEP-34 and TCEP-35.

FIG. 21 shows HIC-HPLC chromatogram of Trastuzumab-[MC-VC-PAB-MMAE]₂ conjugate prepared of Example 66, wherein, the reductant is TCEP-37.

FIG. 22 shows HIC-HPLC chromatogram of Trastuzumab-[MC-VC-PAB-MMAE]₂ conjugate prepared of Comparative example 11.

FIG. 23 shows HIC-HPLC chromatogram of Trastuzumab-[MC-VC-PAB-MMAE]₂ conjugate prepared of Comparative example 12.

FIG. 24 shows HIC-HPLC chromatogram of Trastuzumab-[MC-VC-PAB-MMAE]₂ conjugate prepared of Example 67.

FIG. 25 shows HIC-HPLC chromatogram of Trastuzumab-[MC-VC-PAB-MMAE]2 conjugate prepared of Example 68.

FIG. 26 shows HIC-HPLC chromatogram of Trastuzumab-[MC-VC-PAB-MMAE]₂ conjugate prepared of Example 69.

FIG. 27 shows HIC-HPLC chromatogram of Trastuzumab-[MC-VC-PAB-MMAE]₂ conjugate prepared of Example 70.

FIG. 28 shows HIC-HPLC chromatogram of Trastuzumab-[MC-VC-PAB-MMAE]₂ conjugate prepared of Example 71.

FIG. 29 shows HIC-HPLC chromatogram of Trastuzumab-[MC-VC-PAB-MMAE]₂ conjugate prepared of Example 72.

FIG. 30 shows HIC-HPLC chromatogram of Trastuzumab-[MC-VC-PAB-MMAE]₂ conjugate prepared of Example 73.

FIG. 31 shows HIC-HPLC chromatogram of Trastuzumab-[MC-VC-PAB-MMAE]₂ conjugate prepared of Example 74.

FIG. 32 shows HIC-HPLC chromatogram of Trastuzumab-[MC-VC-PAB-MMAE]₂ conjugate prepared of Example 75.

FIG. 33 shows HIC-HPLC chromatogram of Trastuzumab-[MC-VC-PAB-MMAE]₂ conjugate prepared of Example 76.

FIG. 34 shows HIC-HPLC chromatogram of Trastuzumab-[MC-VC-PAB-MMAE]₂ conjugate prepared of Example 77.

FIG. 35 shows HIC-HPLC chromatogram of Trastuzumab-[MC-VC-PAB-MMAE]₂ conjugate prepared of Example 78.

FIG. 36A-C show HIC-HPLC chromatogram of Trastuzumab-[MC-VC-PAB-MMAE]₂ conjugate prepared of Examples 79-81.

FIG. 37 shows HIC-HPLC chromatogram of Trastuzumab-[MC-VC-PAB-MMAE]₂ conjugate prepared of Comparative Example 10.

FIG. 38A-D show HIC-HPLC chromatogram of Trastuzumab-[MC-VC-PAB-MMAE]₂ conjugate prepared of Examples 82-85, wherein, the molar ratio of the antibody and the reductant is different.

FIG. 39 shows HIC-HPLC chromatogram of Trastuzumab-[MC-VC-PAB-MMAE]₂ conjugate prepared of Example 86.

FIG. 40 shows HIC-HPLC chromatogram of Trastuzumab-[MC-VC-PAB-MMAE]₂ conjugate prepared of Example 87.

FIG. 41 shows HIC-HPLC chromatogram of Trastuzumab-[MC-VC-PAB-MMAE]₂ conjugate prepared of Example 88.

FIG. 42 shows HIC-HPLC chromatogram of Trastuzumab-[MC-VC-PAB-MMAE]₂ conjugate prepared of Example 89.

FIG. 43 shows HIC-HPLC chromatogram of Trastuzumab-[MC-VC-PAB-MMAE]₂ conjugate prepared of Example 90.

FIG. 44 shows HIC-HPLC chromatogram of Trastuzumab-[MC-VC-PAB-MMAE]₂ conjugate prepared of Example 91.

FIG. 45 shows HIC-HPLC chromatogram of Trastuzumab-[MC-VC-PAB-MMAE]₂ conjugate prepared of Example 92.

FIG. 46 shows HIC-HPLC chromatogram of Trastuzumab-[MC-VC-PAB-MMAE]₂ conjugate prepared of Example 93.

FIG. 47 shows HIC-HPLC chromatogram of Trastuzumab-[MC-VC-PAB-MMAE]₂ conjugate prepared of Example 94.

FIG. 48 shows HIC-HPLC chromatogram of Trastuzumab-[MC-VC-PAB-MMAE]₂ conjugate prepared of Example 95.

FIG. 49 shows HIC-HPLC chromatogram of Trastuzumab-[MC-VC-PAB-MMAE]₂ conjugate prepared of Example 96.

FIG. 50 shows HIC-HPLC chromatogram of Trastuzumab-[MC-VC-PAB-MMAE]₂ conjugate prepared of Example 97.

FIG. 51 shows HIC-HPLC chromatogram of Trastuzumab-[MC-VC-PAB-MMAE]₂ conjugate prepared of Example 98.

FIG. 52A-C show HIC-HPLC chromatogram of Trastuzumab-[MC-VC-PAB-MMAE]₂ conjugate prepared of Examples 99-101.

FIG. 53A-C show HIC-HPLC chromatogram of Trastuzumab-[MC-VC-PAB-MMAE]₂ conjugate prepared of Examples 102-104, wherein, the incubation temperature in step (1) is different.

FIG. 54A-D show HIC-HPLC chromatogram of Trastuzumab-[MC-VC-PAB-MMAE]₂ conjugate prepared of Examples 105-108, wherein, the incubation time in step (1) is different.

FIG. 55A-E show HIC-HPLC chromatogram of Trastuzumab-[MC-VC-PAB-MMAE]₂ conjugate prepared of Examples 109-113, wherein, the incubation time in step (1) is different.

FIG. 56 shows HIC-HPLC chromatogram of Trastuzumab-[MC-VC-PAB-MMAE]₂ conjugate prepared of Example 114, wherein, the antibody is engineered antibody.

FIG. 57 shows HIC-HPLC chromatogram of Trastuzumab-[Maleimide-PEG4-N3-DBCO-Cy3]₁ conjugate prepared of Example 115.

FIG. 58 shows HIC-HPLC chromatogram of Trastuzumab-[Maleimide]1 [MC-GGFG-DXd]₆ conjugate prepared of Example 116.

FIG. 59 shows HIC-HPLC chromatogram of Trastuzumab-[MC-MMAF]2 [MC-GGFG-DXd]₆ conjugate prepared of Example 117.

FIG. 60A shows HIC-HPLC chromatogram of Trastuzumab-[MC-VC-PAB-MMAE]₂ conjugate prepared of Example 118; B shows HIC-HPLC chromatogram of Trastuzumab-[MC-VC-PAB-MMAE]2 [MC-GGFG-DXd]2 conjugate prepared of Example 118-119.

FIG. 61 shows HIC-HPLC chromatogram of Trastuzumab-[Maleimide-PEG4-N3-DBCO-Cy3]1 [MC-VC-PAB-MMAE]₂ conjugate prepared of Example 120.

FIG. 62A shows HIC-HPLC chromatogram of Trastuzumab-[Maleimide]1 conjugate prepared of Example 121; B shows HIC-HPLC chromatogram of Trastuzumab-[Maleimide]1 [MC-VC-PAB-MMAE]4 conjugate prepared of Example 121-122.

FIG. 63A shows HIC-HPLC chromatogram of Trastuzumab-[MC-GGFG-DXd]₂ conjugate prepared of Example 123; B shows HIC-HPLC chromatogram of Trastuzumab-[MC-GGFG-DXd]2 [MC-VC-PAB-MMAE]4 conjugate prepared of Example 123-124.

FIG. 64 shows HIC-HPLC chromatogram of Trastuzumab-[Maleimide-PEG4-N3-DBCO-Cy3]1 [MC-VC-PAB-MMAE]4 conjugate prepared of Example 125.

### DETAILED DESCRIPTION

The present disclosure is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following description is intended to illustrate some particular embodiments of the invention, and not to exhaustively specify all permutations, combinations and variations thereof.

Unless defined otherwise, all technical and scientific terms used herein have the same meaning as commonly understood by one of ordinary skill in the art to which the disclosure pertains. Although any methods and materials similar or equivalent to those described herein may be used in the practice for testing of the present disclosure, the preferred materials and methods are described herein. In describing and claiming the present disclosure, the following terminology will be used.

### Reductant

The present disclosure provides examples of the compound which could be acted as a reductant when preparing an antibody with thiol group site-specific modifications.

Provided herein is a compound having the following formula (I):

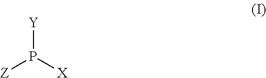

or a salt, solvate, stereoisomer thereof, which characterized in that,
X, Y and Z independently covalently connect the phosphorus atom through P—C bond, which is P—C(sp³) or P—C(sp²);
X is of formula (II)

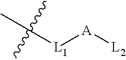

L₁ is selected from the group consisting of —CH(R₁)—, —C(CH₃)(R₁), —CH(R₁)CH (R₂)—, —CH(R₁)CH(R₂)CH(R₃)—, aryl group which is optionally independently substituted with group containing at least a coordinating atom, and heteroaryl group which is optionally independently substituted with group containing at least a coordinating atom;
R₁, R₂ and R₃ independently are H, C₁-C₃ alkyl group, C₁-C₃ hydroxyalkyl group, C₁-C₃ carboxy alkyl group, C₁-C₃ hydroxylamine alkyl group, C₁-C₃ N-hydroxy amide alkyl group, aryl group or heteroaryl group; or
R₂ or R₃ forms a 5-6 membered optionally substituted ring with L₂;
A is optionally present and is —C(O)—, or —C(O)J-;
J is organic group comprising amino or imino group and carbonyl group at the same time, of which the amino or imino group forms amide group with —C(O), the carboxyl group optionally covalently links to L₂;
L₂ is optionally present, L₂ works as transition metal chelator motif and is —N (R₄)(R₅) or hydroxy;
R₄ and R₅ independently are hydrogen, C₀-C₅ hydroxyalkyl group, C₁-C₅ alkyl group, C₁-C₅ alkoxy group, —(CH₂)n¹(OCH₂CH₂O)n²CH(R₆)CO(R₇), optionally substituted 5-6 membered saturated heterocyclic group, optionally substituted arylalkyl group, optionally substituted aryl alkoxy group, optionally substituted aryl group, optionally substituted heteroaryl group, optionally substituted heteroaryl alkyl group, or R₄ and R₅ form a 5-6 membered optionally substituted ring, R₄ or R₅ forms a 5-6 membered optionally substituted ring with R₂ or R₃;
R₆ is hydrogen, amino, C₁-C₃ alkyl, C₁-C₃ hydroxyalkyl group, C₁-C₃ carboxy alkyl group, aryl group, optionally substituted arylalkyl group, C₁-C₃ N-hydroxy amide alkyl group, heteroaryl group or heteroaryl alkyl group;
R₇ is hydroxy, C₁-C₃ alkoxy group, —NH(CH₂CONH)n³OH;
n¹, n² and n³ independently are the number 0, 1, 2, 3, 4;
R₄ and R₅ are not hydroxy at the same time;
Y is same as X,
Z is same as X, or
Y and Z independently are 5-6 membered optionally substituted saturated heterocyclic group, C₁-C₃ alkyl group, C₁-C₃ hydroxyalkyl group, aryl group, C₁-C₃ carboxy alkyl group, 5-6 membered optionally substituted cycloalkyl group, or

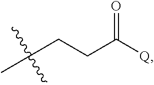

—C(O)Q is ester group, imide group or amide group,
X, Y and Z are not —CH₂CH₂C(O)OH at the same time.

The term “aryl group” refers to an aromatic or hetero aromatic group, composed of one or several rings, comprising three to fourteen carbon atoms, preferentially six to ten carbon atoms. Exemplary aryl group is phenyl group.

The term “aryl group” also refers to an aromatic group, wherein one or several H atoms are replaced independently by other group, such as F, Cl, Br, I, hydroxy, carboxy, sulfonyl, amino, methoxy or ethoxy, N-hydroxy formamide group, N-hydroxy acetamido group, 4-pyridyl group, 2-pyridyl group,

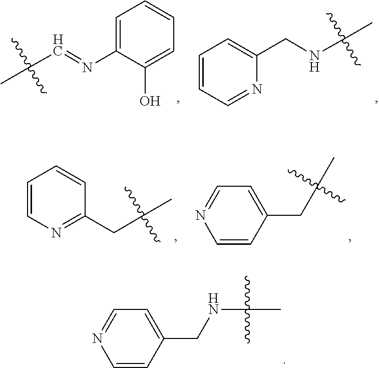

The term “heteroaryl group” refers to one or several carbon on aromatic group, preferentially one, two, three or four carbon atoms are replaced by O, N, Si, Se, P or S, preferentially by O, S, N. Exemplary heteroaryl group is imidazolyl group, pyridyl group, bipyridyl group, quinolinyl group, iso-quinolinyl group.

The term “heteroaryl group” also refers to hetero aromatic group, wherein one or several H atoms are replaced independently by other group, such as F, Cl, Br, I, hydroxy, carboxy, amino, hydroxyalkyl group, carboxy alkyl group, N-hydroxy amide alkyl group, heteroaryl group.

The term “coordinating atom” refers to the atom containing lone paired electron, examples include N, O, S, P, F, Cl, Br, I.

The term “C₁-C₅ alkyl group” refers to an aliphatic hydrocarbon group which having 1 to 3 carbon atoms in the chain or cyclic. Exemplary alkyl groups include methyl, ethyl, n-propyl and i-propyl.

The term “C₀-C₅ hydroxyalkyl group” refers to hydroxy group or C₁-C₅ alkyl group, wherein one or several H atoms are substituted with one, two or three hydroxy groups. Exemplary C₁-C₅ hydroxyalkyl group is hydroxy methyl group, 2-hydroxy ethyl group, 3-hydroxy propyl group.

The term “C₁-C₅ carboxy alkyl group” refers to a C₁-C₅ alkyl group which is substituted with one, two or three carboxy groups. Exemplary C₁-C₅ carboxy alkyl group is —COOH, —CH₂COOH, —CH₂CH₂COOH, —CH₂(CH₃) COOH.

The term “C₁-C₅ hydroxylamine alkyl group” refers to a C₁-C₅ alkyl group which is substituted with one, two or three hydroxylamine groups. Exemplary C₁-C₅ hydroxylamine alkyl group is —CH₂NHOH, —CH₂CH₂NHOH.

The term “C₁-C₅ N-hydroxy amide alkyl group” refers to a C₁-C₅ carboxy alkyl group, wherein one, two or three carboxy forms amide with hydroxylamine. Exemplary C₁-C₅ N-hydroxy amide alkyl group is —C(O)NHOH, —CH₂C(O)NHOH, —CH₂ CH₂C(O)NHOH.

The term “heterocyclic group” refers to an aromatic or non-aromatic C₅-C₁₀ cycle composed of one or two rings, in which one or two of the ring carbon atoms are independently replaced with a heteroatom from the group of O, N, P and S. Preferable heteroatoms are O, N and S. Suitable heterocyclics are also disclosed in The Handbook of Chemistry and Physics, 76*Edition, CRC Press, Inc., 1995-1996, p2-25 to 2-26, the disclosure of which is hereby incorporated by reference. Preferred non aromatic heterocyclic include, but are not limited to pyrrolidinyl, pyrazolidinyl, imidazolidinyl, oxiranyl, tetrahydrofuranyl, dioxolanyl, tetrahydro-pyranyl, dioxanyl, dioxolanyl, piperidyl, piperazinyl, morpholinyl, pyranyl, imidazolinyl, pyrrolinyl, pyrazolinyl, thiazolidinyl, tetrahydrothiopyranyl, dithianyl, thiomorpholinyl, dihydro-pyranyl, tetrahydropyranyl, diliydropyranyl, tetrahydro-pyridyl, dihydropyridyl, tetrahydropyrinidinyl, dihydrothiopyranyl, a/epanyl, as well as the fused systems resulting from the condensation with a phenyl group.

The term “arylalkyl group” refers to a liner, branched or cycloalkyl which is linked to at least one aryl group. Preferable the number of carbon atoms in the chain or cyclic is 1-4. Exemplary arylalkyl group is —CH₂C₆H₅, —CH₂CH₂C₆H₅, —CH₂CH₂CH₂C₆H₅, —CH₂ (CH₃)CH₂C₆H₅, —CH₂(CH₃)CH₂CH₂C₆H₅.

The term “heteroaryl alkyl group” refers to a liner, branched or cycloalkyl which is linked to at least one heteroaryl group. Preferable the number of carbon atoms in the chain or cyclic is 1-4. Exemplary heteroaryl alkyl group is

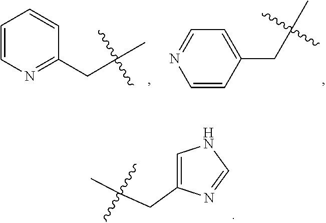 [SMILES: *Cc1ccccn1 ; *Cc1ccncc1 ; *Cc1c[nH]cn1]

The term “C₁-C₅ alkoxy group” refers to an oxygen atom attached to C₁-C₅ alkyl group. Exemplary C₁-C₅ alkoxy group is —OCH₃, —OCH₂CH₃, —OCH₂(CH₃)₂, —OCH₂ CH₂CH₃.

The term “aryl alkoxy group” refers to an aromatic group, wherein one or several H atoms are replaced by alkoxy group. Exemplary phenyl-O—CH₂—, phenyl-O—(CH₂)₂—, phenyl-O—(CH₂)₃—, phenyl-O—(CH₂)₄—, phenyl-O—(CH₂)₅—.

The term “cycloalkyl group” refers to 3-, 4-, 5- or 6-membered saturated or unsaturated non-aromatic carbocyclic ring. Representative cycloalkyl group include, but are not limited to, cyclopropyl, cyclobutyl, cyclopentyl, cyclopentadienyl, cyclohexyl, cyclohexenyl, 1, 3-cyclohexadienyl, 1, 4-cyclohexadienyl. Cycloalkyl group can be unsubstituted or substituted with one or more groups including, but not limited to carboxyl, sulfonyl, amino, hydroxy, —C(O)NHOH, —CH₂C(O)NHOH, —CH₂ CH₂C(O)NHOH, —COOH, —CH₂COOH, —CH₂CH₂COOH, —CH₂(CH₃) COOH, F, Cl, Br, I.

The term “halogen” refers to F, Cl, Br or I.

The term “Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted and may be straight or branched.

The term “Cyano” refers to a substituent having a carbon atom joined to a nitrogen atom by a triple bond, e.g., —CN.

In some embodiments, L₁ is —CH(R₁)—, —CH(R₁)CH(R₂)— or —CH(R₁)CH(R₂)CH(R₃)—. In some embodiments, L₁ is —CH(R₁)CH(R₂)—.

In some embodiments, R₁, R₂ and R₃ independently are H, methyl group, isopropyl group, hydroxymethyl group, hydroxyethyl group, carboxy methyl group, carboxy ethyl group, N-hydroxy ethyl amide group, phenyl group, 2-pyridyl group, 4-pyridyl group or 4-imidazole group, R₂ forms a 5-6 membered optionally substituted ring with L₂.

In some embodiments, L₁ is —CH(R₁)CH(R₂)—, R₁ is H, and R₂ forms a 5-6 membered optionally substituted ring with R₄ of L₂. In some embodiments, R₂ forms

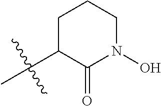 [SMILES: *C1CCCN(O)C1=O]

with L₂. In these embodiments, A is —C(O)—, L₂ is —N(R₄)(R₅), R₅ is hydroxy.

In some embodiments, L₁ is —CH(R₁)CH(R₂)—, R₁ is methyl group, isopropyl group, carboxy ethyl group or N-hydroxy ethyl amide group, R₂ is H.

In some embodiments, L₁ is —CH(R₁)CH(R₂)—, R₁ is methyl group, isopropyl group, carboxy ethyl group or N-hydroxy ethyl amide group, R₂ is H, A is —C(O)—, L₂ is —N (R₄)(R₅), R₄ is hydrogen, and R₅ is hydroxy.

In some embodiments, L₁ is —CH(R₁)CH(R₂)—, R₁ is H, R₂ is methyl group, hydroxymethyl group, hydroxyethyl group, carboxy ethyl group, phenyl group, N-hydroxy ethyl amide group, 2-pyridyl group, 4-pyridyl group or 4-imidazole group.

In some embodiments, L₁ is —CH(R₁)CH(R₂)—, R₁ is H, R₂ is methyl group, hydroxymethyl group, hydroxyethyl group, carboxy ethyl group, phenyl group, N-hydroxy ethyl amide group, 2-pyridyl group, 4-pyridyl group or 4-imidazole group, A is —C(O)—, L₂ is —N (R₄)(R₅), R₄ is hydrogen, optionally substituted 5-6 membered saturated heterocyclic group, R₅ is hydroxy. In these embodiments, R₄ is

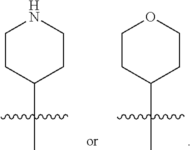 [SMILES: *C1CCNCC1 ; *C1CCOCC1]

In some embodiments, L₁ is —CH(R₁)CH(R₂)—, R₁ is H, R₂ is methyl group, hydroxymethyl group, hydroxyethyl group, carboxy ethyl group, phenyl group, N-hydroxy ethyl amide group, 2-pyridyl group, 4-pyridyl group or 4-imidazole group, A is —C(O)—, L₂ is —N (R₄)(R₅), R₄ and R₅ form a 5-6 membered optionally substituted ring. In these embodiments, L₂ is

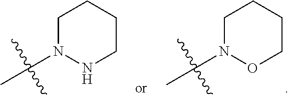 [SMILES: *N1CCCCN1 ; *N1CCCCO1]

In some embodiments, L₁ is optionally substituted phenyl group connected to A in ortho, meta or para position, A is —C(O)—; L₂ is —N (R₄)(R₅) or hydroxy; R₄ is hydrogen, R₅ is hydroxy.

In some embodiments, L₁ is phenyl group which is optionally substituted with hydroxy, halogen, carboxyl, sulfonyl, amino, methoxy or ethoxy in ortho, meta or para position. In these embodiments, A and L₂ are not present. Halogen refers to F, Cl, Br or I.

In some embodiments, L₁ is

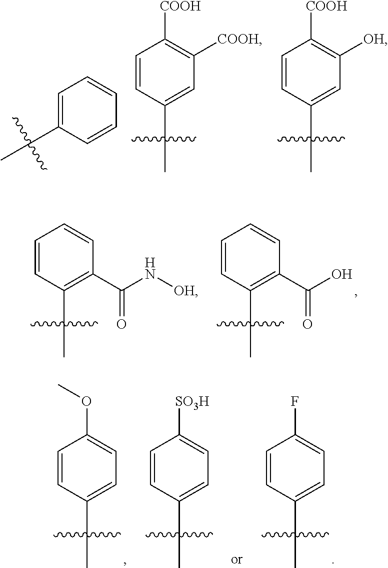

In these embodiments, A and L2 are not present.

In some embodiments, L₁ is optionally substituted 4-pyridyl group
or optionally substituted 4-quinolyl group. In some embodiments, L₁ is

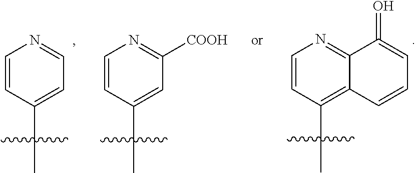

In these embodiments, A and L₂ are not present.

In some embodiments, L₁ is —CH(R₁)CH(R₂)—, R₁ and R₂ independently are H.

In some embodiments, A is —C(O)—, L₂ is —N (R₄)(R₅), R₄ is hydrogen, R₅ is hydroxy.

In some embodiments, L₂ is —N (R₄)(R₅), R₄ is hydrogen, C₁-C₅ alkyl group, —(CH₂) n¹(OCH₂CH₂O)n²CH(R₆)CO(R₇), optionally substituted 5-6 membered saturated heterocyclic group, optionally substituted arylalkyl group, optionally substituted aryl group, optionally substituted heteroaryl alkyl group, or R₄ and R₅ form a 5-6 membered optionally substituted ring; R₅ is hydroxy.

In some embodiments, L₂ is —N (R₄)(R₅), R₄ is hydrogen, C₁-C₅ alkyl group, —(CH₂) n¹(OCH₂CH₂O)n²CH(R₆)CO(R₇), optionally substituted arylalkyl group, optionally substituted aryl group; R₅ is hydroxy.

In some embodiments, R₆ is hydrogen, amino, C₁-C₅ alkyl, C₁-C₅ hydroxyalkyl group, C₁-C₅ carboxy alkyl group, aryl group, C₁-C₅ N-hydroxy amide alkyl group, heteroaryl group or heteroaryl alkyl group.

In some embodiments, R₆ is hydrogen, C₁-C₅ alkyl, C₁-C₅ hydroxyalkyl group, or heteroaryl alkyl group.

In some embodiments, R₆ is hydrogen, methyl group, hydroxymethyl group amino, benzyl group, carboxy ethyl group, N-hydroxy ethyl amide group,

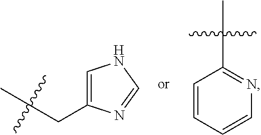 [SMILES: *Cc1c[nH]cn1 ; *c1ccccn1]

In some embodiments, R₇ is hydroxy, C₁-C₅ alkoxy group, —NH(CH₂CONH)n³OH, optionally, R₇ is hydroxy or C₁-C₅ alkoxy group. In some embodiments, R₇ is hydroxy, methoxy group, —NH(CH₂CONH)n³OH, optionally, R₇ is hydroxy or methoxy group.

In some embodiments, n¹, n² and n³ independently are the number 0, 1, 2, 3, 4.

In some embodiments, R₄ is

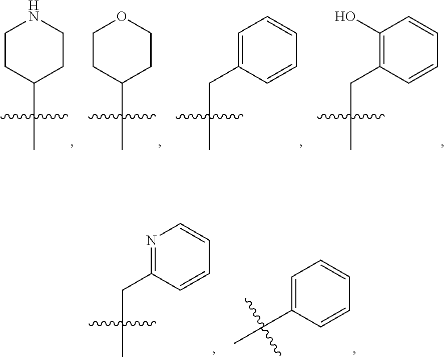

hydrogen or —(CH₂)n¹(OCH₂CH₂O)n²CH(R₆)CO(R₇), R₅ is hydroxy,
R₆ is hydrogen, methyl group, hydroxymethyl group or

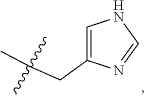 [SMILES: *Cc1c[nH]cn1]

R₇ is hydroxy or —NH(CH₂CONH)n³OH;
n¹, n² and n³ independently are the number 0.

In some embodiments, R₄ is

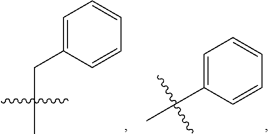 [SMILES: *Cc1ccccc1 ; *c1ccccc1]

hydrogen or —CH(R₆)CO(R₇), R₅ is hydroxy,
R₆ is hydrogen,
R₇ is hydroxy.

In some embodiments, L₂ is —N (R₄)(R₅);
R₄ and R₅ are independently —(CH₂)n¹(OCH₂CH₂O)n²CH(R₆)CO(R₇) or optionally substituted heteroaryl alkyl group,
R₆ is hydrogen, amino, C₁-C₅ alkyl, C₁-C₅ hydroxyalkyl group, C₁-C₅ carboxy alkyl group, aryl group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4.

In some embodiments, R₄ and R₅ are independently —(CH₂)n¹(OCH₂CH₂O)n²CH (R₆)CO(R₇) or 6 membered heteroaryl alkyl group,
R₆ is hydrogen,
R₇ is hydroxy, C₁-C₅ alkoxy group, —NH(CH₂CONH)n³OH;
n¹, n² and n³ independently are the number 0, 1, 2, 3, 4.

In some embodiments, R₄ and R₅ are independently —(CH₂)n¹(OCH₂CH₂O)n²CH (R₆)CO(R₇) or

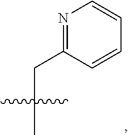 [SMILES: *Cc1ccccn1]

R₆ is hydrogen,
R₇ is hydroxy or —NH(CH₂CONH)n³OH;
n¹, n² and n³ independently are the number 0.

In some embodiments, R₄ and R₅ are independently —(CH₂)n¹(OCH₂CH₂O)n²CH (R₆)CO(R₇),
R₆ is hydrogen,
R₇ is hydroxy or —NH(CH₂CONH)n³OH;
n¹, n² and n³ independently are the number 0.

In some embodiments, L₂ is —N (R₄)(R₅);
R₄ is hydrogen, C₀-C₅ hydroxyalkyl group, C₁-C₅ alkyl group, optionally substituted C₁-C₅ alkoxy group, —(CH₂)n¹(OCH₂CH₂O)n²CH(R₆)CO(R₇), optionally substituted arylalkyl group, optionally substituted aryl alkoxy group, optionally substituted aryl group, optionally substituted heteroaryl group, optionally substituted heteroaryl alkyl group;
R₅ is hydrogen,
R₆ is hydrogen, amino, C₁-C₅ alkyl, C₁-C₅ hydroxyalkyl group, C₁-C₅ carboxy alkyl group, aryl group, optionally substituted arylalkyl group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4.

In some embodiments, R₄ is hydrogen, C₀-C₃ hydroxyalkyl group, C₁-C₃ alkoxy group, —(CH₂)n¹(OCH₂CH₂O)n²CH(R₆)CO(R₇), phenyl group which is substituted with carboxy, hydroxy, amino, halogen, pyridyl group, amino which is substituted with 2-methylpyridine, benzyl group which is substituted with carboxy, hydroxy, amino or halogen, aryl alkoxy group, pyridyl group which is substituted with carboxy, bipyridyl group,

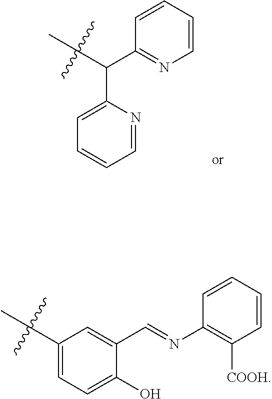 [SMILES: *C(c1ccccn1)c1ccccn1 ; *c1ccc(O)c(/C=N/c2ccccc2C(=O)O)c1]

In some embodiments, R₄ is hydrogen, hydroxy, methyl hydroxyl group, ethyl hydroxyl group, propyl hydroxyl group, methoxy group, ethoxy group,

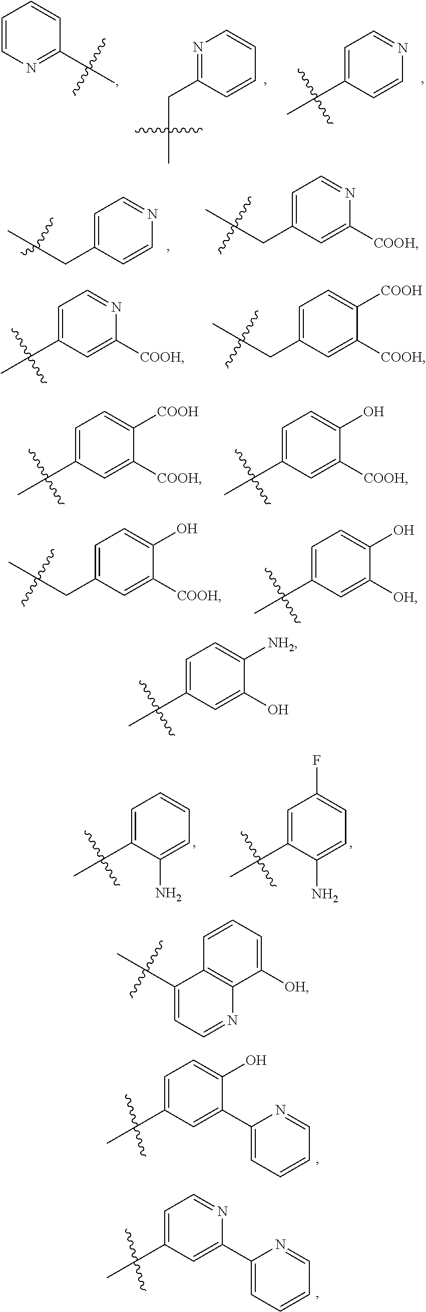

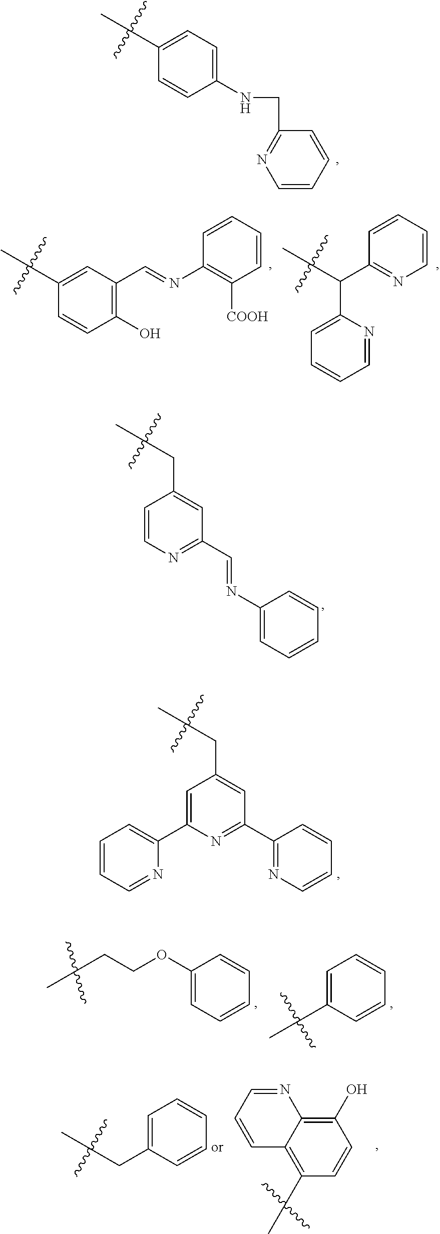

and R₅ is hydrogen.

In some embodiments, R₄ is hydroxy, methoxy group, or

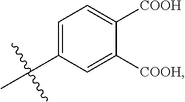 [SMILES: *c1ccc(C(=O)O)c(C(=O)O)c1]

and R₅ is hydrogen.

In some embodiments, R₄ is —(CH₂)n¹(OCH₂CH₂O)n²CH(R₆)CO(R₇), R₅ is hydrogen, R₆ is hydrogen, amino, C₁-C₃ alkyl, C₁-C₃ hydroxyalkyl group, C₁-C₃ carboxy alkyl group, aryl group, arylalkyl group which is optionally substituted with hydroxyl group, halogen, cyano group or nitro group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4.

In some embodiments, R₄ is —(CH₂)n¹(OCH₂CH₂O)n²CH(R₆)CO(R₇), R₅ is hydrogen, R₆ is hydrogen, amino, methyl, hydroxymethyl group, benzyl group, benzyl group substituted with hydroxyl group, halogen, cyano group or nitro group, halogen, carboxy ethyl group, N-hydroxy ethyl amide group,

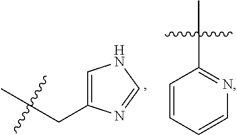

R₇ is hydroxyl, —NH(CH₂CONH)n³OH;
n¹ and n³ independently are the number 0, 1, 2, 3, 4,
n² is the number 0.

In some embodiments, R₄ is —(CH₂)n¹(OCH₂CH₂O)n²CH(R₆)CO(R₇), R₅ is hydrogen, R₆ is hydrogen, amino, methyl, hydroxymethyl group, benzyl group, carboxy ethyl group,

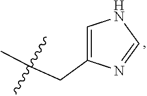 [SMILES: *Cc1c[nH]cn1]

N-hydroxy ethyl amide group,

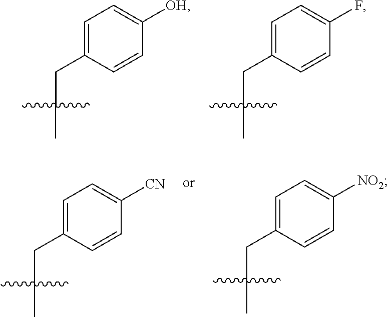 [SMILES: *Cc1ccc(O)cc1 ; *Cc1ccc(F)cc1 ; *Cc1ccc(C#N)cc1 ; *Cc1ccc([N+](=O)[O-])cc1]

R₇ is hydroxyl or —NH(CH₂CONH)n³OH;
n¹ is the number 0 or 2,
n² is the number 0 or 1,
n³ is the number 0.

In some embodiments, R₄ is —(CH₂)n¹(OCH₂CH₂O)n²CH(R₆)CO(R₇), R₅ is hydrogen, R₆ is hydrogen; R₇ is —NH(CH₂CONH)n³OH; n¹, n² and n³ are the number 0.

In some embodiments, A is —C(O)J-, J is peptide residue, comprising mono amino acid residue, dipeptide, tripeptide, tetrapeptide, pentapeptide, aminopropionic acid, aminobutyric acid, amino valeric acid, aminoacid, aminoheptanoic acid, aminooctanoic acid, or NH₂ (OCH₂CH₂O)n⁴CH₂COOH, n⁴ is the number of 2-10.

the amino acid is selected from the group consisting of glycine (Gly), alanine (Ala), serine (Ser), arginine (Arg), asparagine (Asn), asparticacid (Asp), cysteine (Cys), glutamine (Gln), glutamicacid (Glu), histidine (His), isoleucine (Ile), leucine (Leu), lysine (Lys), methionine (Met), phenylalanine (Phe), proline (Pro), threonine (Thr), tryptophan (Trp), tyrosine (Tyr) and valine (Val).

In some embodiments, J is the residue of histidine, serine, alanine, glycine, phenylalanine, asparagine, tyrosine or asparagine.

In some embodiments, A is —C(O)J-, J is the residue of histidine, serine, alanine, glycine, phenylalanine, asparagine, tyrosine or asparagine, L₂ is —N (R₄)(R₅), R₄ is hydrogen, R₅ is hydroxy.

In some embodiments, Y is same as X.

In some embodiments, Z is same as X.

In some embodiments, Y and Z independently are

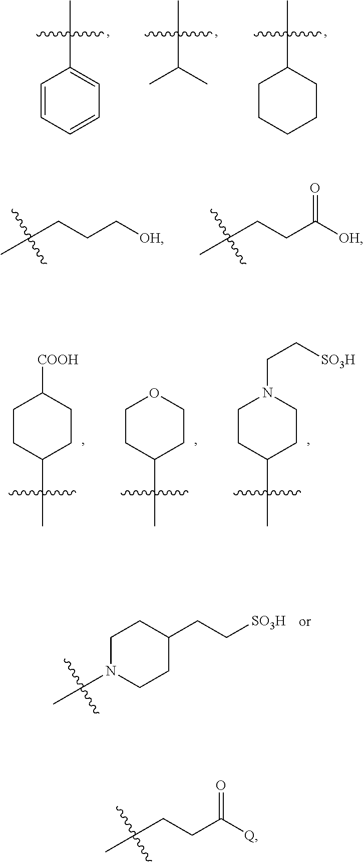

Q is —NHOH, —NHCH₂CH₂SO₃H, —N(CH₂CH₂OH)₂, —NHCH₂COOH, —NHCH(CH₃) COOH, —NH(CH₂CH₂O)₃CH₃.

In some embodiments, without the limitation, the compound is selected from the group consisting of

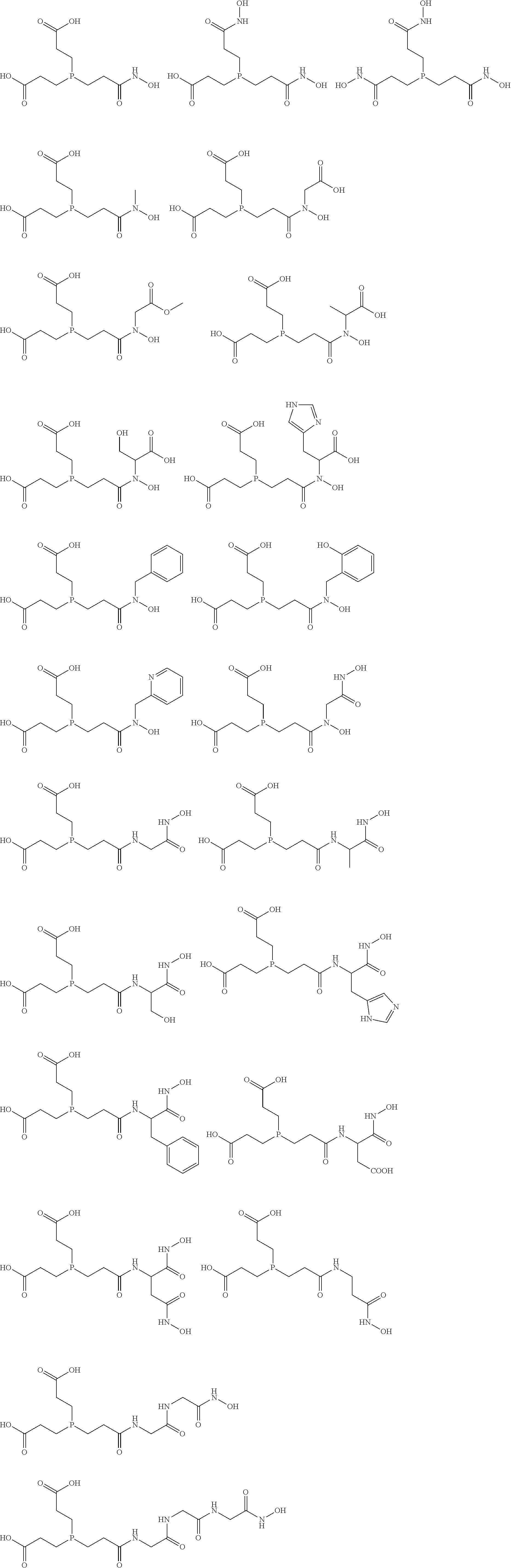

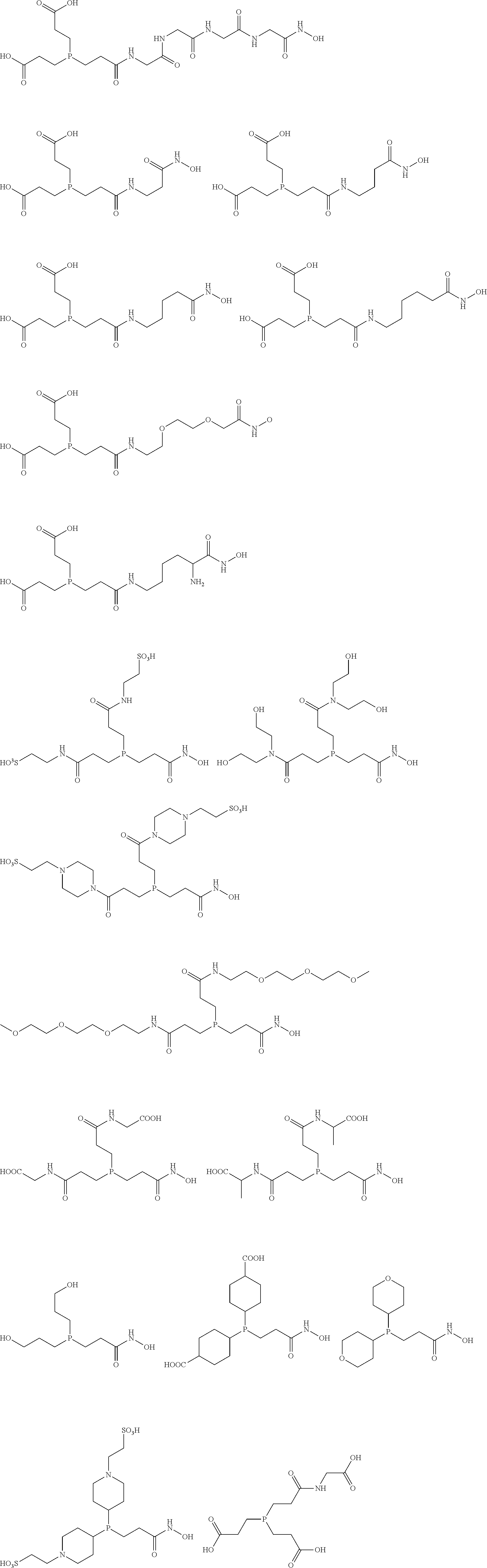

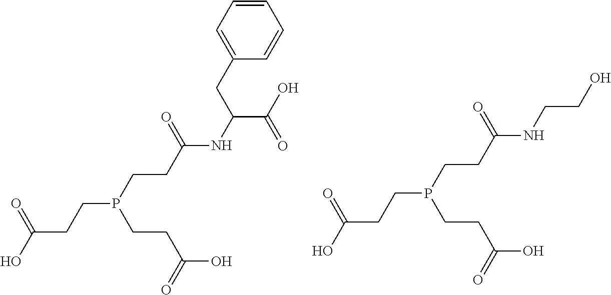 [SMILES: O=C(O)CCP(CCC(=O)O)CCC(=O)NC(Cc1ccccc1)C(=O)O ; O=C(O)CCP(CCC(=O)O)CCC(=O)NCCO]

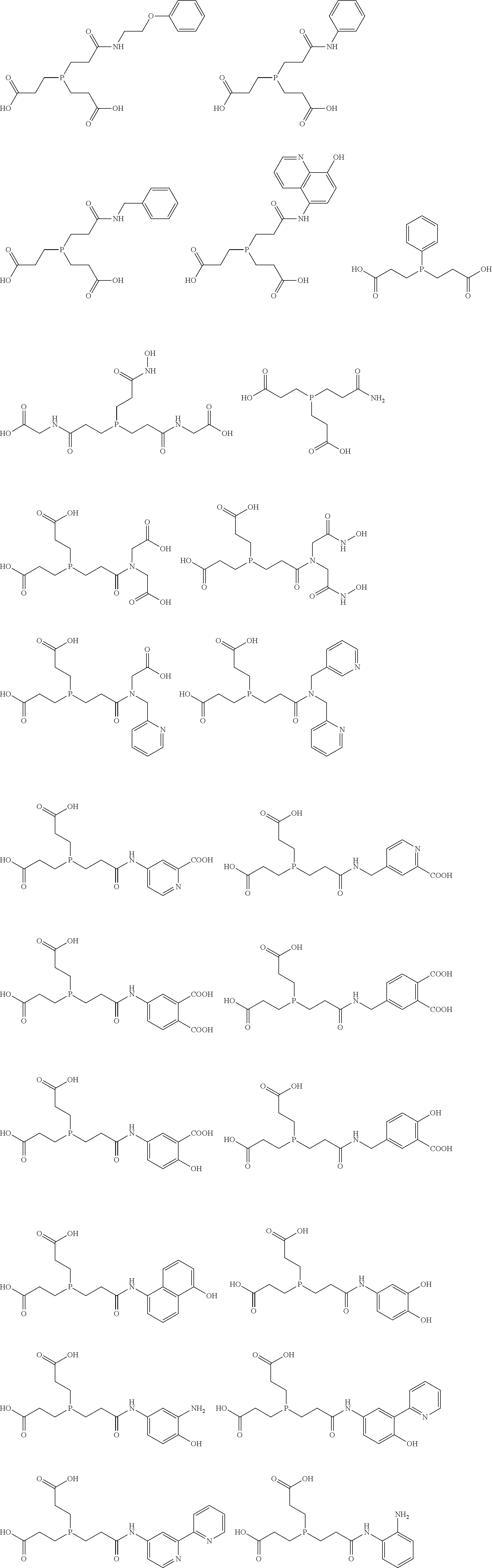

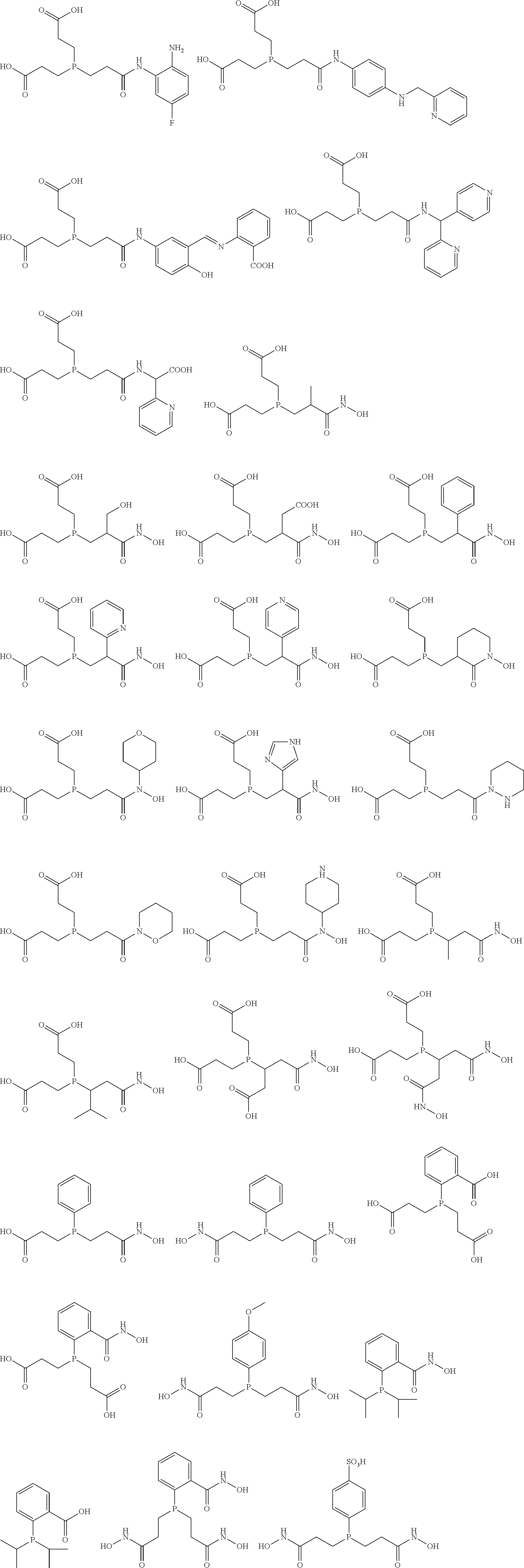

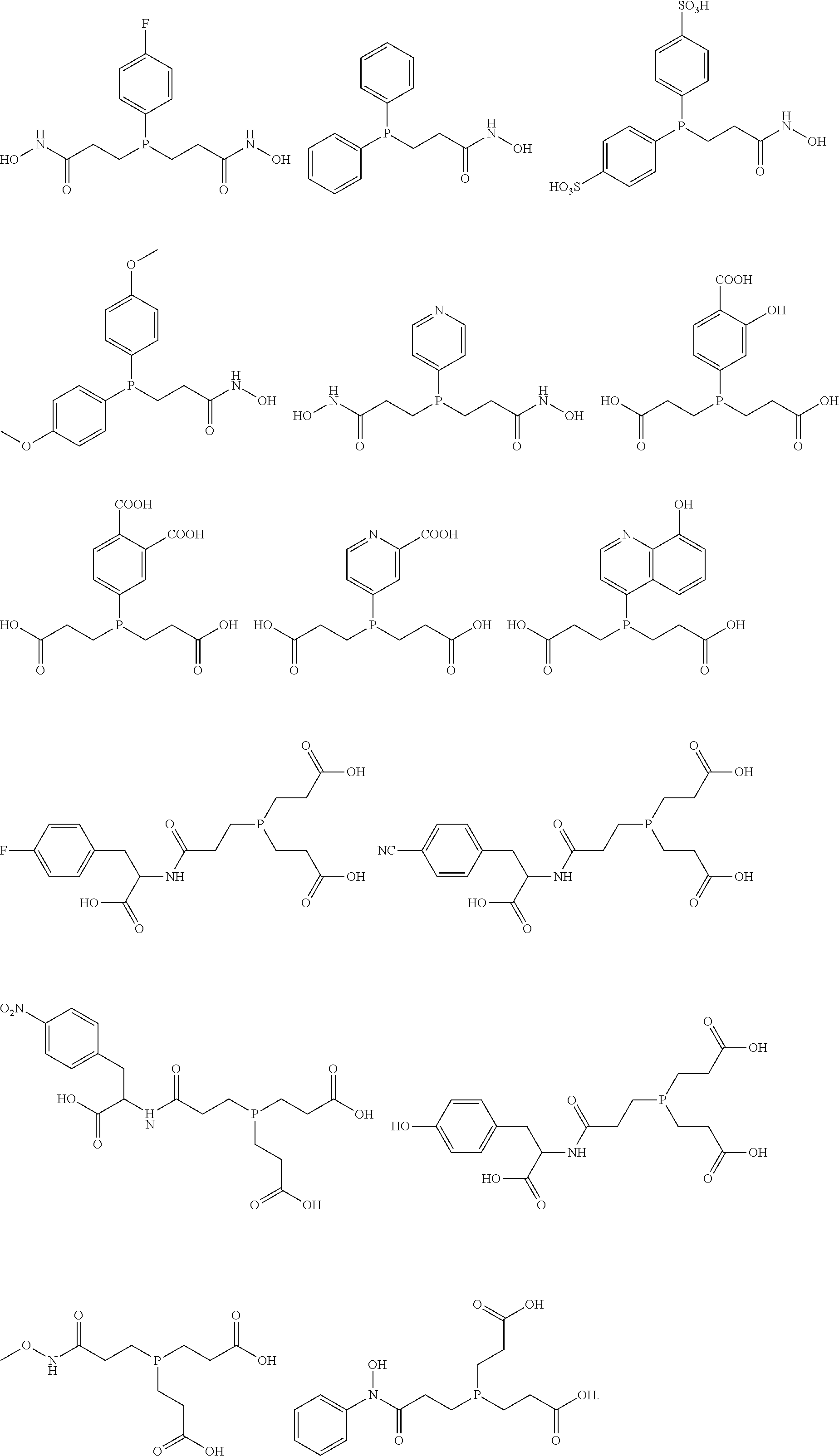

The disclosure provides a composition comprising the compound described above and transition metal ions.

In some embodiments, the transition metal ion is Zn²⁺, Cd²⁺, Hg²⁺, Ni²⁺, Co²⁺ or combination thereof. In some embodiments, the transition metal ion is Zn²⁺.

In some embodiments, the molar ratio of the compound described above and the transition metal ions is 1:0.4 to 1:250, 1:0.4 to 1:200, 1:0.4 to 1:60 or 1:6 to 1:16.

The compound having formula (I) described above could be prepared as the following steps:
at least one carboxyl group of following formula III is connected to the heteroatom of a transition metal chelator moiety

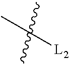

by introducing a condensation reagent under an inert atmosphere,

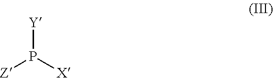

wherein X′ is

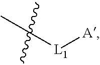

L₁ is selected from the group consisting of —CH(R₁)—, —C(CH₃)(R1), —CH(R₁) CH(R₂)—, —CH(R₁)CH(R₂)CH(R₃)—, aryl group which is optionally substituted with group or groups containing N, O or S, and heteroaryl group which is optionally substituted with group or groups containing O or S;
R₁, R₂ and R₃ indepently are H, C₁—C alkyl group, C₁-C₅ hydroxyalkyl group, C₁-C₅ carboxy alkyl group, C₁-C₅ hydroxylamine alkyl group, C₁-C₅ N-hydroxy amide alkyl group, aryl group or heteroaryl group; or
R₂ or R₃ forms a 5-6 membered optionally substituted ring with L₂;
A′ is —COOH or —C(O)J-COOH;
J is organic group comprising amino or imino group and carbonyl group at the same time, of which the amino or imino group forms amide group with —C(O), the carboxyl group optionally covalently linked to L₂;
L₂ is optionally present, L₂ works as transition metal chelator motif and is —N(R₄)(R₅) or hydroxy;
R₄ and R₅ independently are hydrogen, C₀-C₅ hydroxy alkyl group, C₁-C₅ alkyl group, C₁-C₅ alkoxy group, —(CH₂)n¹(OCH₂CH₂O)n²CH(R₆)CO(R₇), optionally substituted 5-6 membered saturated heterocyclic group, optionally substituted arylalkyl group, optionally substituted aryl alkoxy group, optionally substituted aryl group, optionally substituted heteroaryl group, optionally substituted heteroaryl alkyl group, or R₄ and R₅ form a 5-6 membered optionally substituted ring, R₄ or R₅ forms a 5-6 membered optionally substituted ring with R₂ or R₃;
R₆ is hydrogen, amino, C₁-C₅ alkyl, C₁-C₅ hydroxyalkyl group, C₁-C₅ carboxy alkyl group, aryl group, optionally substituted arylalkyl group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4;
R₄ and R₅ are not hydroxy at the same time;
Y′ is same as X′;
Z′ is same as X′, or
Y′ and Z′ independently are 5-6 membered optionally substituted saturated heterocyclic group, C₁-C₅ alkyl group, C₁-C₅ hydroxyalkyl group, aryl group, C₁-C₅ carboxy alkyl group, 5-6 membered optionally substituted cycloalkyl group, or

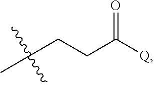

—C(O)Q is ester group, imide group or amide group.

In some embodiments, the transition metal chelator moiety can be provided by 2-phenoxy-ethylamine, Phenylamine, Benzylamine, 4-Aminobenzene-1, 2-diol, 5-Amino-2-hydroxybenzoic acid, Bis (pyridin-2-ylmethyl) amine, 5-Amino-8-hydroxyquinoline, Bis (pyridin-2-yl) methanamine, 4-Aminophthalic acid, tert-Butyl L-tyrosinate, DL-3-(4-Fluorophenyl) alanine, DL-4-Cyanophenylalanine, DL-4-nitro-phenylalanine, N-Benzylhydroxylamine hydrochloride, N-Phenylhydroxylamine,

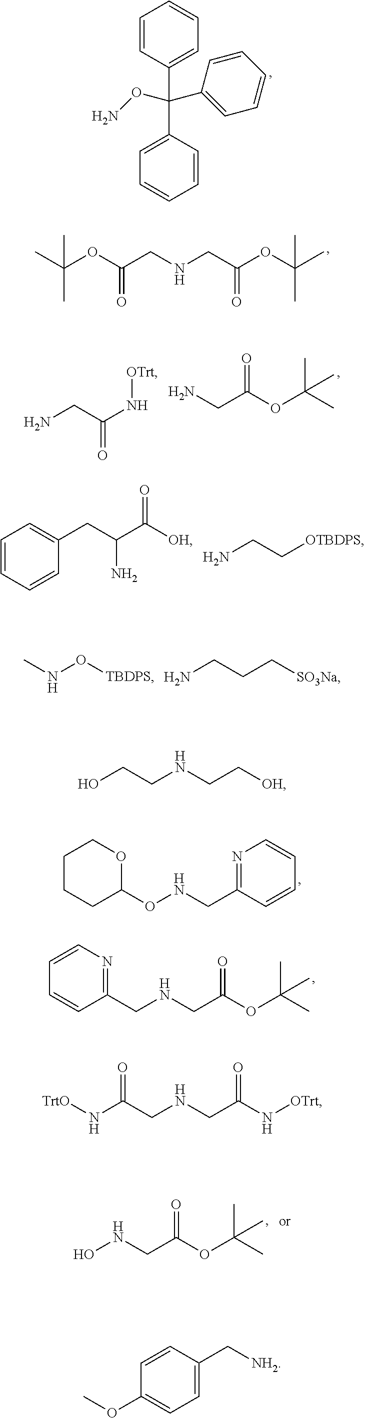

In some embodiments, the structure of formula III is

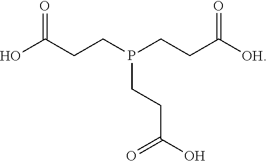 [SMILES: O=C(O)CCP(CCC(=O)O)CCC(=O)O]

The term “condensation reagent” refers to a condensation reaction reagent, which helps two molecules (functional groups) combine covalently to form one single molecule. Condensation reagent includes, but not limited to 1-Hydroxybenzotriazole (HOBT), O-Benzotriazole-N, N, N′, N′-tetramethyl-uronium-hexafluorophosphate (HBTU), and O-(Benzotriazol-1-yl)-N, N, N′, N′-tetramethyluronium tetr afluoroborate (TBTU).

The term “inert atmosphere” refers to the chemically inactive atmosphere, such as nitrogen, carbon dioxide, helium.

Use in Manufacture of an Antibody with Thiol Group Site-Specific Modifications

The compound having formula (I) provided above has reducibility and could reduce the disulfide bond of an antibody, thus the compound having formula (I) can act as a reductant in the process of protein modification or antibody modification.

As used herein, the term “disulfide bond” refers to a covalent bond with the structure R—S—S—R′. The amino acid cysteine comprises a thiol group that can form a disulfide bond with a second thiol group, for example from another cysteine residue. The disulfide bond can be formed between the thiol groups of two cysteine residues residing respectively on the two polypeptide chains, thereby forming an interchain bridge or interchain bond.

In some embodiments, the compound having formula (I) could reduce the interchain S-Sbonds of an antibody.

In some embodiments, the compound having formula (I) could selectively reduce one of the interchain S-Sbonds, thus the antibody is selectively modified.

In some embodiments, the compound having formula (I) provided above could act as a reductant in the preparation of an antibody with thiol group site-specific modifications, optionally, the antibody with thiol group site-specific modifications is antibody drug conjugate (ADCs).

A mixture of antibody-drug conjugates will be generated by the conventional conjugation processes or the bio-conjugation process of the present disclosure. In general, one antibody molecule belonging to IgG1 or IgG4 subclass has 4 inter-chain S-Sbonds, each of which is formed with two —SH groups. The antibody molecule can be subjected to partial or complete reduction of one or more interchain S-Sbonds to form 2n (n is an integer selected from 1, 2, 3 or 4) reactive —SH groups, and thus, the number of drugs (or payloads) coupling to a single antibody molecule is 1, 2, 3, 4, 5, 6, 7 or 8. In accordance with the number of drugs coupling to a single antibody molecule, the different conjugates containing different number and/or kinds of drug molecules are denominated as D0, D2, D1, D4, D6, D8, D1+D6, D1+D3, D2+D6, D2+D3, D0+D6, D0+D3, the ADC with D0+D4, the ADC with D2+D4, the ADC with D1+D4, the ADC with D2+D2 or the ADC with D1+D2. And thus, the “homogeneity” of antibody-drug conjugates is used to describe the property of dominance of one specific type of antibody-drug conjugate (i.e., one type selected from D0, D1, D2, D4, D6, D8, D1+D6, D1+D3, D2+D6, D2+D3, D0+D6 or D0+D3 conjugates) in one given mixture of antibody-drug conjugates.

Drug to Antibody Ratio (DAR) of ADC is the average number of drugs linked to each antibody. DAR is a key property used to measures the quality of ADC because it can significantly affect ADC efficacy. The DAR distribution (D0, D2, D4, D6, D8) could reflect the homogeneity of the ADC.

Drug loading is represented by the number of drug moieties per antibody in a molecule of ADC. For some antibody-drug conjugates, the drug loading may be limited by the number of attachment sites on the antibody. For example, where the attachment is a cysteine thiol, as in certain exemplary embodiments described herein, the drug loading may range from 0 to 8 drug moieties per antibody.

As used herein, the term “D0” or “the ADC with D0” refers to the ADC in which the number of drugs coupling to a single antibody molecule is about zero.

As used herein, the term “D2” or “the ADC with D2” refers to DAR about 2, it means about two drug molecules (e.g., 1.5, 2.0, 2.5 molecules) are coupled to one single antibody molecule. Drug molecules may be coupled to —SH groups generated by reduction of disulfide bond between heavy and light chains or heavy and heavy chains via linkers.

As used herein, the term “D4” or “the ADC with D4” refers to the ADC in which about four drug molecules (e.g., 3.5, 4.0, 4.5 molecules) are coupled to one single antibody molecule, where the drug molecules may be coupled to four —SH groups generated by reduction of two interchain disulfide bonds or intrachain disulfide bonds.

As used herein, the term “D6” or “the ADC with D6” refers to the ADC in which about six drug molecules (e.g., 5.5, 6.0, 6.5 molecules) are coupled to one single antibody molecule, where the drug molecules may be coupled to six —SH groups generated by reduction of three disulfide bond.

As used herein, the term “D8” or “the ADC with D8” refers to the ADC in which about eight drug molecules (e.g., 7.5, 8.0, 8.5 molecules) are coupled to one single antibody molecule, where the drug molecules may be coupled to eight —SH groups generated by reduction of four disulfide bond.

As used herein, the term “D1” or “the ADC with D1” refers to the ADC in which one of the first thiobridge group bearing the first linker-payload re-bridges two thiol groups of one single antibody molecule.

As used herein, the term “D3” or “the ADC with D3” refers to the ADC in which three of the first thiobridge group bearing the first linker-payload re-bridges six thiol groups of one single antibody molecule.

As used herein, the term “D1+D6” or “the ADC with D1+D6” refers to the ADC in which one of the first thiobridge group bearing the first linker-payload re-bridging two thiol groups and six of the second linker-payloads are coupled to one single antibody molecule, wherein, the first linker-payload and the second linker-payload may be same or different.

As used herein, the term “D1+D3” or “the ADC with D1+D3” refers to the ADC in which one of the first thiobridge group bearing the first linker-payload and three of the second thiobridge groups bearing the second linker-payload re-bridge eight thiol groups of one single antibody molecule, wherein, the first thiobridge group and the second thiobridge group may be same or different, and the first linker-payload and the second linker-payload may be same or different.

As used herein, the term “D2+D6” or “the ADC with D2+D6” refers to the ADC in which two of the first linker-payloads and six of the second linker-payloads are coupled to one single antibody molecule, wherein, the first linker-payload and the second linker-payload may be same or different.

As used herein, the term “D2+D3” or “the ADC with D2+D3” refers to the ADC in which two of the first linker-payloads are coupled to one single antibody molecule and three of the second thiobridge groups bearing the second linker-payload re-bridging six thiol groups of the antibody, wherein, the first linker-payload and the second linker-payload may be same or different.

As used herein, the term “D0+D6” or “the ADC with D0+D6” refers to the ADC in which one of the first thiobridge group re-bridging two thiol groups and six of the second linker-payloads are coupled to one single antibody molecule, or refers to the ADC in which two of the end capping reagents and six of the second linker-payloads are coupled to one single antibody molecule.

As used herein, the term “D0+D3” or “the ADC with D0+D3” refers to the ADC in which one of the first thiobridge group re-bridges two thiol groups and three of the second thiobridge group bearing the linker-payload re-bridge six thiol groups of one single antibody molecule, wherein, the first thiobridge group and the second thiobridge group may be same or different. In some embodiments, D0+D3” refers to the ADC in which two of the end capping reagents react with two thiol groups and three of the second thiobridge group bearing the linker-payload re-bridge six thiol groups of one single antibody molecule.

As used herein, the term “D0+D4” or “the ADC with D0+D4” refers to the ADC in which one of the first thiobridge group re-bridges two thiol groups and four of the second linker-payloads are coupled to one single antibody molecule, or refers to the ADC in which two of the end capping reagents and four of the second linker-payloads are coupled to one single antibody molecule.

As used herein, the term “D1+D4” or “the ADC with D1+D4” refers to the ADC in which one of the first thiobridge group bearing the first linker-payload re-bridges two thiol groups and four of the second linker-payloads are coupled to one single antibody molecule.

As used herein, the term “D2+D4” or “the ADC with D2+D4” refers to the ADC in which two of the first linker-payloads and four of the second linker-payloads are coupled to one single antibody molecule.

As used herein, the term “D2+D2” or “the ADC with D2+D2” refers to the ADC in which two of the first linker-payloads and two of the second linker-payloads are coupled to one single antibody molecule.

As used herein, the term “D1+D2” or “the ADC with D1+D4” refers to the ADC in which one of the first thiobridge group bearing the first linker-payload re-bridges two thiol groups and two of the second linker-payloads are coupled to one single antibody molecule.

As used herein, the term “D4+D2” or “the ADC with D4+D2” refers to the ADC in which four of the first linker-payloads and two of the second linker-payloads are coupled to one single antibody molecule.

As used herein, the term “D4+D4” or “the ADC with D4+D4” refers to the ADC in which four of the first linker-payloads and four of the second linker-payloads are coupled to one single antibody molecule.

As used herein, the term “homogeneity of the ADC with Dx” refers to that the weight content of the ADC with Dx in all the ADCs produced by the method, wherein, Dx maybe D1, D2, D1+D6, D1+D3, D2+D6, D2+D3, D0+D6, D0+D3, D0+D4, D2+D4, D1+D4, D2+D2 or D1+D2.

As used herein, the term “about” refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In particular embodiments, the terms “about” when preceding a numerical value indicates the value plus or minus a range of 50%, 30%, 15%, 10%, 5%, or 1%.

In some embodiments, the compound having formula (I) provided above or the composition provided above could be used to prepare ADC with improved homogeneity.

In some embodiments, the disclosure provides the use of compound having formula (I) or the composition provided above in the preparation of ADC with D2, the ADC with D1, the ADC with D2+D6, the ADC with D2+D3, the ADC with D1+D6, the ADC with D1+D3, the ADC with D0+D6, the ADC with D0+D3, the ADC with D0+D4, the ADC with D2+D4, the ADC with D1+D4, the ADC with D2+D2 or the ADC with D1+D2.

In some embodiments, the ADC comprises D2 in a content at least up to 53% of the total weight of D0, D2, D4, D6 and D8 combined. In some embodiments, the ADC comprises D2 in a content up to 55% of the total weight of D0, D2, D4, D6 and D8 combined. In some embodiments, the ADC comprises D2 in a content up to 60%, 65%, 70%, 75%, 80%, 84%, 87%, 89%, 90%, 91%, 92% or 95% of the total weight of D0, D2, D4 and D8 combined.

In some embodiments, the homogeneity of the ADC with D1, the ADC with D2+D6, the ADC with D0+D6 is up to 80%.

In some embodiments, the content of the ADC with D2+D2 is generally up to 68% or 70%.

In some embodiments, the content of the ADC with D1+D2 is generally up to 80% or 83%.

In some embodiments, the content of the ADC with D0+D4 is generally up to 55%, 61% or 65%.

In some embodiments, the content of the ADC with D2+D4 is generally up to 70%, 75%, even to 78% or 80%.

In some embodiments, the content of the ADC with D1+D4 is generally up to 60%, 65%, even to 70%.

Method of Preparing an Antibody with Thiol Group Site-Specific Modifications

The present disclosure also provides the method of preparing the antibody with thiol group site-specific modifications, the thiol group (s) is/are reduced from the interchain disulfide bonds within the antibody, and the method comprises using the compound or a salt, solvate, stereoisomer thereof described above and the transition metal ions or using the composition described above.

In some embodiments, the number of the thiol group (s) is/are 1, 2, 3, 4, 5, 6, 7 or 8.

In some embodiments, the number of the thiol groups is 2 or 8.

In some embodiments, the interchain disulfide bonds connected the two upper heavy chains in the hinge region, or the heavy chain to the light chain in the Fab region.

In some embodiments, the interchain disulfide bonds connected the two heavy chains in the hinge region, and the heavy chain to the light chain in the Fab region.

In some embodiments, the site-specific modification dose not refer to antibody technologies, enzyme technologies and glycan modification.

In some embodiments, the method comprises the following steps:
(a) incubating the compound or a salt, solvate, stereoisomer thereof described above which works as a first reductant and the antibody in the presence of the transition metal ions in a first buffer system to selectively reduce the interchain disulfide bonds within the antibody; or
incubating the composition described above, wherein the compound described above works as the first reductant, and the antibody in the first buffer system to selectively reduce the interchain disulfide bonds within the antibody;
(b) introducing metal chelators and a modification reagent 1 to react with the reduced thiol groups resulted from step (a), wherein, the modification reagent 1 is an end capping reagent, a first linker-payload or a first thiobridge reagent, optionally, the first thiobridge reagent bears the first linker-payload or reactive groups.

In some embodiments, when the first thiobridge reagent bears the reactive groups, the step (b) comprises the following step:
introducing metal chelators and the first thiobridge reagent bearing reactive groups to re-bridge the reduced thiol groups resulted from step (a), then, incubating the first linker-payload in the first buffer system to react with the reactive groups of the thiobridge group.

In some embodiments, the method further comprises the following steps,
(c) incubating the reaction product from step (b) and a second reductant in a second buffer system to reduce the interchain disulfide bonds in the reaction product, optionally, introducing the transition metal ions;
(d) introducing the incubation product from step (c) and a modification reagent 2 to react with the reduced thiol groups resulted from step (c), optionally, introducing the metal chelators, wherein, the modification reagent 2 is a second linker-payload or a second thiobridge reagent, optionally, the second thiobridge reagent bears the second linker-payload or reactive groups.

In some embodiments, when the second thiobridge reagent bears the reactive groups, the step (d) comprises the following steps:
introducing the reaction product from step (c) and the second thiobridge reagent bearing reactive groups to re-bridge the reduced thiol groups resulted from step (c), optionally, introducing the metal chelators, then, incubating the second linker-payload in the second buffer system to react with the reactive groups of the thiobridge group.

In some embodiments, when introducing the transition metal ions in step (c), introducing the metal chelators to trap the excess transition metal ions in step (d).

As used herein, the term “bear”, “bears” or “bearing” refers to have or having.

In some embodiments, at first, the first reductant reduces one of the interchain disulfide bond within the antibody selectively with the transition metal ions, optionally, the second reductant reduces the remaining three interchain disulfide bonds without the transition metal ions, or the second reductant reduces one or two of the interchain disulfide bonds with the transition metal ions. The antibody with thiol group site-specific modifications, such as the ADC with D1 or the ADC with D2, could be prepared by the method including the step (a) and (b). The antibody with thiol group site-specific modifications, such as the ADC with D1+D6, the ADC with D1+D3, the ADC with D2+D6, the ADC with D2+D3, the ADC with D0+D6, the ADC with D0+D3, the ADC with D0+D4, the ADC with D2+D4, the ADC with D1+D4, the ADC with D2+D2 or the ADC with D1+D2, could be prepared by the method including the step (a), (b), (c) and (d).

In some embodiments, the salt refers to acid addition salts or base addition salts.

In some embodiments, acid addition salts can be formed with inorganic acids and organic acids. The inorganic acids from which salts can be derived include, for example, hydrochloric acid, hydrobromic acid, sulfuric acid, phosphoric acid, and the like. The organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

In some embodiments, base addition salts can be formed with inorganic bases and organic bases. The inorganic bases from which salts can be derived include groups 1 to 2 of the periodic table. In certain embodiments, the salts are derived from lithium, sodium, potassium, calcium, magnesium and the like. The organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In some embodiments, the first reductant and the transition metal ions are used together in the method of prepare the antibody with thiol group site-specific modifications. In some embodiments, the molar ratio of the first reductant and the transition metal ions is 1:0.4 to 1:250. In some embodiments, the molar ratio of the first reductant and the transition metal ions is 1:0.4 to 1:200. In some embodiments, the molar ratio of the first reductant and the transition metal ions is 1:0.4 to 1:60. In some embodiments, the molar ratio of the first reductant and the transition metal ions is 1:1 to 1:60. The molar ratio of the first reductant and the transition metal ions is 1:2 to 1:60. In some embodiments, the molar ratio of the first reductant and the transition metal ions is 1:6 to 1:16. In some embodiments, the molar ratio of the first reductant and the transition metal ions is 1:190, 1:1 to 1:180, 1:170, 1:160, 1:1 to 1:150, 1:140, 1:1 to 1:130, 1:120, 1:1 to 1:100, 1:1 to 1:80, or 1:1 to 1:70.

In some embodiments, the molar ratio of the first reductant and the antibody is 3:1 or 0.5:1 or 3:1 to 1:1. In some embodiments, the molar ratio of the first reductant and the antibody is 2:1 to 1:1. In some embodiments, the molar ratio of the first reductant and the antibody is 1:1, 1.5:1, 1.8:1, 2:1, 2.5:1, 2.8:1 or 3:1.

In some embodiments, there is no specific limitation to the concentration of the first reductant, as long as scaling up or down the concentration of the transition metal ions and the antibody in equal proportions. In some embodiments of the present applications, the concentration of the first reductant is 0.01 mM to 0.2 mM. In some embodiments of the present applications, the concentration of the first reductant is 0.02 mM to 0.15 mM. In some embodiments of the present applications, the concentration of the first reductant is 0.05 mM to 0.1 mM. In some embodiments of the present applications, the concentration of the first reductant is 0.01 mM, 0.02 mM, 0.03 mM, 0.04 mM, 0.05 mM, 0.06 mM, 0.07 mM, 0.08 mM, 0.09 mM, 0.10 mM, 0.11 mM, 0.12 mM, 0.13 mM, 0.14 mM, 0.15 mM, 0.16 mM, 0.17 mM, 0.18 mM, 0.19 mM or 0.20 mM.

In some embodiments, there is no specific limitation to the concentration of the transition metal ions in step (a), as long as scaling up or down the concentration of the first reductant and the antibody in equal proportions.

In some embodiments of the present application, there is no specific limitation to the concentration of the antibody in step (a), as long as scaling up or down the concentration of the first reductant and the transition metal ions in equal proportions.

The reductant selectively reduces disulfide bonds in the first buffer system, the first buffer system and the second buffer system are independently selected from a group consisting of HEPES buffer, Histidine buffer, PBS, PB, MES buffer, BES buffer, MOPS buffer, Bis-Tris buffer, Acetate buffer, DIPSO buffer, MOPSO buffer, TES buffer, ACES buffer, MOBS buffer, TAPSO buffer, ADA buffer, PIPES buffer, BTP buffer, HEPPSO buffer, POPSO buffer, EPPS buffer or Tris buffer.

As used herein, the term “HEPES buffer” refers to 4-hydroxyethyl piperazine ethanesulfonic acid buffer.

As used herein, the term “PBS” refers to phosphate buffer saline.

As used herein, the term “PB’ refers to phosphate buffer.

As used herein, the term “MES buffer” refers to 2-(N-morpholino) ethanesulfonic acid buffer.

As used herein, the term “BES buffer” refers to N, N-Bis (2-hydroxyethyl)-2-aminoethanesulphonic acid buffer.

As used herein, the term “MOPS buffer” refers to 3-morpholinopropanesulfonic Acid buffer.

As used herein, the term “Bis-Tris buffer” refers to Bis (2-hydroxyethyl) amino-tris (hydroxymethyl) methane buffer.

As used herein, the term “DIPSO buffer” refers to 3-[bis(2-hydroxyethyl) amino]-2-hydroxypropanesulphonic acid buffer.

As used herein, the term “MOPSO buffer” refers to 3-(N-morpholino)-2-hydroxy-1-propanesulfonic acid buffer.

As used herein, the term “TES buffer” refers to 2-[tris (hydroxymethyl) methylamino]-1-ethanesulfonic acid buffer.

As used herein, the term “ACES buffer” refers to N-(carbamoylmethyl) taurine buffer.

As used herein, the term “MOBS buffer” refers to 4-(N-morpholino) butanesulfonic Acid buffer.

As used herein, the term “TAPSO buffer” refers to 3-[N-tris-(hydroxymethyl) methylamino]-2-hydroxypropanesulphonic acid buffer.

As used herein, the term “ADA buffer” refers to N—(Carbamoylmethyl) iminodiacetic acid buffer.

As used herein, the term “PIPES buffer” refers to piperazine-1, 4-bisethanesulfonic acid buffer.

As used herein, the term “BTP buffer” refers to Bis-tris propane buffer.

As used herein, the term “Heppso buffer” refers to N—(Hydroxyethyl) piperazine-N′-2-hydroxypropanesulfonicacid buffer.

As used herein, the term “POPSO buffer” refers to piperazine-N, N′-bis(2-hydroxy-propane sulfonic) acid buffer.

As used herein, the term “EPPS buffer” refers to 4-(2-Hydroxyethyl)-1-piperazinepropanesulfonic acid buffer.

As used herein, the term “Tris buffer” refers to tris (hydroxymethyl) aminomethane buffer.

In some embodiments, the first buffer system and the second buffer system are independently selected from a group consisting of Bis-Tris buffer, PIPES buffer, MOPS buffer, BES buffer, HEPES buffe, ADA buffer, PB, DIPSO buffer, MOBS buffer, MOPSO buffer, TES buffer, ACES buffer, TAPSO buffer or MES buffer.

In some embodiments, the first buffer system and the second buffer system are MES buffer.

In some embodiments, the concentration of the first buffer system and the second buffer system is 10-100 mM (mmol/L).

In some embodiments, the pH value of the first buffer system and the second buffer system is 5.5 to 8.0. In some embodiments, the pH value of the buffer system is 5.8 to 8.0. In some embodiments, the pH value of the first buffer system and the second buffer system is 6.0 to 7.4. In some embodiments, the pH value of the first buffer system and the second buffer system is 6.7 to 7.4. In some embodiments, the pH value of the first buffer system and the second buffer system is 5.8, 6.0, 6.2, 6.5, 6.8, 7.0, 7.2 or 7.4.

In some embodiments, the first buffer system and the second buffer system are MES buffer and the pH value of MES buffer is 5.8 to 6.7.

The term “transition metal ions” refers to the elements of groups 4-12, justified by their typical chemistry, i.e., a large range of complex ions in various oxidation states, colored complexes, and catalytic properties either as the element or as ions (or both). Sc and Y in Group 3 are also generally recognized as transition metals.

In some embodiments, the transition metal ions are selected from a group consisting of Zn²⁺, Cd²⁺, Hg²⁺, Ni²⁺, Co²⁺ or the combination thereof.

In some embodiments, the transition metal ion is Zn²⁺.

In some embodiments, there is no specific limitation to the salts of the transition metal ions, as long as the transition metal ions are soluble in the reaction solution so that free transition metal ions can be released in the reaction solution. In some embodiments, the salts of the transition metal ions are chloride, nitrate, sulfate, acetate, iodide, bromine, formate or tetrafluorborate.

In some embodiments, the salts of Zn²⁺ are ZnCl₂, Zn (NO₃) 2, ZnSO₄, Zn (CH₃COO) 2, ZnI₂, ZnBr₂, Zinc formate, or zinc tetrafluoroborate. In some embodiments of the present application, the salts of Zn²⁺ are ZnCl₂.

Those skilled in the art should understand that the incubation temperature and incubation time in step (a) depend on specific antibodies to be conjugated. In some embodiments, the incubation temperature is 0° C. to 37° C., 0° C. to 25° C. or 0° C. to 15° C. in step (a), the incubation time is 0.2 h to 24 h in step (a), optionally, the incubation temperature is 0° C. to 10° C. in step (a), and the incubation time is 2 h to 16 h in step (a).

In some embodiments, the incubation temperature is 0° C. to 15° C., 0° C. to 10° C., 0° C. to 8° C., 0° C. to 6° C. in step (a). In some embodiments, the incubation temperature is 4° C., 8° C., 12° C., 15° C., 18° C., 24° C., 30° C., 35° C. or 37° C. in step (a).

In some embodiments, the incubation time is 0.5 h to 24 h, 0.5 h to 20 h, 0.5 h to 16 h, 0.5 h to 12 h, 0.5 h to 8 h or 0.5 h to 6 h in step (a). In some embodiments, the incubation time is 0.25 h, 0.3 h, 0.5 h, 0.7 h, 1 h, 1.5 h, 2 h, 3 h, 4 h, 5 h, 6 h, 7 h, 10 h, 12 h, 14 h, 16 h, 18 h, 20 h, 22 h or 24 h.

In some embodiments, the molar ratio of the first reductant and the antibody is 2:1 to 3:1, the incubation time is 0.5 h to 9 h in step (a). In some embodiments, the molar ratio of the first reductant and the antibody is 2.8:1 to 3:1, the incubation time is 1 h to 9 h in step (a). In some embodiments, the molar ratio of the first reductant and the antibody is 2.2:1, 2.4:1, 2.6:1, 2:8.1 or 3:1, the incubation time is 0.5, 1 h, 2 h, 3 h, 4 h, 5 h, 6 h, 7 h, 8 h, 9 h or 9.5 h in step (a).

In some embodiments, the incubation temperature is 0° C. to 25° C. in step (a), the incubation time is 0.5 h to 24 h in step (a). In some embodiments, the incubation temperature is 0° C. to 15° C. in step (a), the incubation time is 0.5 h to 24 h in step (a). In some embodiments, the incubation temperature is 0° C. to 10° C. in step (a), the incubation time is 2 h to 16 h in step (a).

In some embodiments, in step (c), there is no specific limitation to the second reductant, as long as the second reductant could reduce the interchain disulfide bonds within the antibody. In some embodiments, the second reductant is the same as the second reductant. In some embodiments, the second reductant is TCEP, Tris (3-hydroxypropyl) phosphine (THPP), or Dithiothreitol (DTT). In some embodiments, the second reductant is TCEP.

In some embodiments, in step (c), without the transition metal ions, there is no specific limitation to concentration of the second reductant, as long as the second reductant could reduce the interchain disulfide bonds within the antibody completely. In some embodiments of the present application, the molar ratio of the second reductant and the antibody is 3:1 to 20:1, 3:1 to 10:1, 4:1 to 10:1, 5:1 to 9:1, 6:1 to 9:1, 6:1 to 8:1. In some embodiments, the molar ratio of the second reductant and the antibody is 20:3.

In some embodiments, the incubation time of the second reductant is 0.5 h to 24 h, or 5 h to 20 h in step (c). In some embodiments, the incubation time of the second reductant is 6 h to 18 h, 8 h to 18 h, 8 h to 15 h, or 8 h to 12 h in step (c). In some embodiments, the incubation time of the second reductant is 8 h or 12 h in step (c).

In some embodiments, in step (c), introducing the transition metal ions, two of the interchain disulfide bonds are selectively reduced. In some embodiments, in step (c), the molar ratio of the second reductant and the transition metal ions is 1:0.05 to 1:40, and/or the molar ratio of the second reductant and the antibody is 2.5:1 to 20:1, and/or the incubation time is 1 h to 24 h. In some embodiments, in step (c), the molar ratio of the second reductant and the transition metal ions is 1:0.05, 1:0.08, 1:0.1, 1:0.2, 1:0.3, 1:0.4, 1:0.5, 1:0.6, 1:0.7, 1:0.8, 1:0.9, 1:1, 1:2, 1:4, 1:6, 1:8, 1:10, 1:12, 1:14, 1:16, 1:18 or 1:20. In some embodiments, in step (c), the molar ratio of the second reductant and the antibody is 2.5:1, 3:1, 5:1, 7:1, 9:1, 11:1, 13:1, 15:1, 17:1, 19:1 or 20:1. In some embodiments, in step (c), the incubation time is 1 h, 2 h, 4 h, 6 h, 8 h, 10 h, 12 h, 14 h, 16 h, 18 h, 20 h, 22 or 24 h. In some embodiments, in step (c), the molar ratio of the second reductant and the transition metal ions is 1:0.05 to 1:40, and/or the molar ratio of the second reductant and the antibody is 3:1 to 15:1, and the incubation time is 1 h to 12 h. In some embodiments, in step (c), the molar ratio of the second reductant and the transition metal ions is 1:0.05 to 1:40, and/or the molar ratio of the second reductant and the antibody is 2.5:1 to 15:1, and the incubation time is 12 to 24 h.

In some embodiments, in step (c), introducing the transition metal ions, one of the interchain disulfide bonds are selectively reduced. In some embodiments, in step (c), the molar ratio of the second reductant and the transition metal ions is 1:0.5 to 1:100, and/or the molar ratio of the second reductant and the antibody is 0.8:1 to 2.5:1, and/or the incubation time is 0.5 h to 24 h. In some embodiments, in step (c), the molar ratio of the second reductant and the transition metal ions is 1:0.5, 1:1, 1:4, 1:8, 1:12, 1:24, 1:30, 1:40, 1:50, 1:50, 1:70, 1:80, 1:90, 1:100. In some embodiments, in step (c), the molar ratio of the second reductant and the antibody is 0.8:1, 1:1, 1.2:1, 1.4:1, 1.6:1, 1.8:1, 2:1, 2.2:1, 2.4:1, or 2.5:1. In some embodiments, in step (c), the incubation time is 0.5 h, 1 h, 2 h, 4 h, 6 h, 8 h, 10 h, 12 h, 14 h, 16 h, 18 h, 20 h, 22 or 24 h. In some embodiments, in step (c), the molar ratio of the second reductant and the transition metal ions is 1:0.5 to 1:100, and/or the molar ratio of the second reductant and the antibody is 0.8:1 to 2:1, and the incubation time is 0.5 h to 24 h. In some embodiments, in step (c), the molar ratio of the second reductant and the transition metal ions is 1:0.5 to 1:100, and/or the molar ratio of the second reductant and the antibody is 2:1 to 2.5:1, and the incubation time is 1 h to 9 h.

In some embodiments, the incubation temperature of the second reductant is 0° C. to 37° C., or 5° C. to 30° C. in step (c). In some embodiments, the incubation temperature of the second reductant is 10° C. to 30° C., 15° C. to 30° C., 20° C. to 30° C., or 25° C. to 30° C. in step (c). In some embodiments, the incubation temperature of the second reductant is 25° C. in step (c).

In some embodiments, in step (b) and in step (d), the reaction temperature with the reduced thiol groups is 4° C. to 40° C., 10° C. to 40° C., 10° C. to 35° C., 10° C. to 30° C., 10° C. to 25° C., 15° C. to 35° C., 20° C. to 30° C., 4° C. to 37° C., 20° C. to 30° C. or 20° C. to 25° C. In some embodiments, in step (b) and in step (d), the reaction temperature with the reduced thiol groups is 24° C.

In some embodiments, in step (b) and in step (d), the reaction time with the reduced thiol groups is 0.5 h to 6 h, 0.5 h to 5 h, 0.5 h to 4 h, 0.5 h to 3 h, 0.5 h to 3 h, 0.5 h to 2 h, 0.5 h to 1 h. In some embodiments, in step (b) and in step (d), the reaction time with the reduced thiol groups is 0.5 h, 1 h, 2 h or 3 h.

In some embodiments, the reactive temperature and time with the reduced thiol groups in step (b) and step (d) are independent.

In some embodiments, in step (b) and in step (d), the reaction temperature with the reactive groups is 10° C. to 37° C., 20° C. to 30° C., 10° C. to 30° C., 15° C. to 30° C. or 25° C. to 30° C. In some embodiments, in step (b) and in step (d), the reaction temperature with the reactive groups is 25° C.

In some embodiments, in step (b) and in step (d), the reaction time with the reactive groups is 2 h to 12 h, 2 h to 10 h, 4 h to 10 h, 6 h to 10 h, or 8 h to 10 h. In some embodiments, in step (b) and (d), the reaction time with the reactive groups is 8 h.

In some embodiments, the reactive temperature and time with the reactive groups in step (b) and step (d) are independent.

In some embodiments, the metal chelators can trap excessive said transition metal ions in step (b). In some embodiments, there is no specific limitation to the metal chelators, as long as the metal chelators can trap the excessive transition metal ions and do not affect the reduction of the disulfide bonds within the antibody. In some embodiments, the metal chelators are selected from a group consisting of ethylene diamine tetraacetic acid (EDTA), nitrilotriacetic acid (NTA), diethylenetriaminepentaacetic acid (DTPA), citric Acid (CA), tartaric acid (TA), gluconic acid (GA) or N-(2-hydroxyethyl) ethylenediamine-N, N′, N′-triacetic acid (HEDTA).

In some embodiments, the metal chelators are selected from a group consisting of EDTA, NTA or DTPA. In some embodiments, the metal chelators are EDTA.

In some embodiments, the molar ratio of the metal chelators and the antibody in step (b) is 1:1 to 100:1, 10:1 to 100:1, 20:1 to 100:1, 20:1 to 80:1, 20:1 to 70:1, 30:1 to 60:1, 40:1 to 50:1, 35:1 to 60:1, 40:1 to 55:1.

In some embodiments, the molar ratio of the metal chelators and the antibody in step (d) is 1:1 to 100:1, 1:1 to 60:1, 1:1 to 50:1, 1:1 to 20:1, 1:1 to 10:1, 1:1 to 8:1, 1:1 to 6:1, 1:1 to 5:1, 2:1 to 8:1, 2:1 to 6:1.

In some embodiments, the excess amount of metal chelators and a complex of the metal chelators and the transition metal ions are filtered out in dialysis, ultrafiltration or gel filtration.

In some embodiments, in step (b), according to the amount of the antibody, the modification reagent 1 is excess.

In some embodiments, in step (b), the molar ratio of the first thiobridge reagent and the antibody is 5:1 to 1:1, 2:1 to 1:1, 1.5:1 to 1:1, 1.2:1 to 1:1 or 1.1:1 to 1:1. In some embodiment, in step (b), the molar ratio of the first thiobrige reagent and the antibody is 1.05:1.

In some embodiments, in step (b), when the first linker-payload reacts with the reduced thiol groups, the molar ratio of the first linker-payload and the antibody is 5:1 to 1:1, 2:1 to 10:1, 3:1 to 10:1, 4:1 to 9:1 or 5:1 to 7:1. In some embodiments, in step (b), when the first linker-payload reacts with the reduced thiol groups, the molar ratio of the first linker-payload and the antibody is 5:1.

In some embodiments, in step (b), when the first linker-payload reacts with the reactive groups in the first thiobridge reagent, the molar ratio of the first linker-payload and the antibody is 5:1 to 1:1, 4:1 to 1:1, 3:1 to 1:1 or 2:1 to 1:1. In some embodiments, in the step (b), the molar ratio of the first linker-payload and the antibody is 5:3.

In some embodiments, in step (d), according to the amount of the antibody, the modification reagent 2 is excess.

In some embodiments, in step (d), the molar ratio of the second thiobridge reagent and the antibody is 5:1 to 1:1, 5:1 to 3:1, 4:1 to 3:1, 4:1 to 3.2:1 or 4:1 to 3.5:1. In some embodiments, in step (b), the molar ratio of the second thiobridge reagent and the antibody is 5:1, 4.5:1, 4:1, 3.8:1, 3.5:1 or 3.2:1.

In some embodiments, in step (d), when the second linker-payload reacts with the reduced thiol groups, the molar ratio of the second linker-payload and the antibody is 20:1 to 2:1, 20:1 to 6:1, 18:1 to 8:1, 16:1 to 8:1, 14:1 to 8:1, 12:1 to 10:1. In some embodiments, in step (d), when the second linker-payload reacts with the reduced thiol groups, the molar ratio of the second linker-payload and the antibody is 35:3.

In some embodiments, in step (d), when the second linker-payload reacts with the reactive groups in the second thiobridge reagent, the molar ratio of the second linker-payload and the antibody is 10:1 to 1:1, 10:1 to 2:1, 10:1 to 3:1, 9:1 to 3:1, 8:1 to 3:1, 7:1 to 3:1, 6:1 to 3: 1, 5:1 to 3:1 or 4:1 to 3:1.

In some embodiments, said method further comprises the following steps:

optionally, introducing a compound that contains at least one thiol group to consume excessive said first linker-payload in step (b) and/or said second linker-payload in step (d);

purifying and recovering the resultant antibody with thiol group site-specific modifications in step (b) and/or in step (d).

In some embodiments, there is no specific limitation to a compound to consume excessive said first linker-payload and/or said second linker-payload, as long as the compound contains at least one thiol group. In some embodiments, the compound is cysteine.

By purifying and recovering the resultant antibody with thiol group site-specific modifications in step (b) and/or in step (d), the content of the antibody with thiol group site-specific modifications could be higher. In some embodiments, the resultant antibody with thiol group site-specific modifications is purified by a de-salting column, size exclusion chromatography, ultrafiltration, dialysis and/or the like. In some embodiments, the resultant antibody with thiol group site-specific modifications is purified by a de-salting column. If needed, further enrichment (e.g., D2) may be applied in some case using hydrophobic interaction chromatography (HIC).

In some embodiments, there is no specific limitation to the antibody. According to the antigens associated with the disease, those skilled in the art can select suitable antibody useful in the bio-conjugation process of the present application. In some embodiments, the antibody is a monoclonal antibody, a polyclonal antibody, a mono-specific antibody or a multi-specific antibody.

As used herein, the term “antibody” refers to any immunoglobulin that binds to a specific antigen. A native intact antibody comprises two heavy chains and two light chains. Each heavy chain consists of a variable region and a first, second, and third constant region, while each light chain consists of a variable region and a constant region. The heavy chain from any vertebrate species can be assigned to one of five different classes (or isotypes): IgA, IgD, IgE, IgG, and IgM.

In some embodiments, the antibody is a human antibody, a humanized antibody, a chimeric antibody or an antigen-binding moiety thereof.

As used herein, the term “human antibody” refers to one which possesses an amino acid sequence which corresponds to that of an antibody produced by a human or a human cell or derived from anon-human source that utilizes human antibody repertoires or other human antibody-encoding sequences. This definition of a human antibody specifically excludes a humanized antibody comprising non-human antigen-binding residues.

As used herein, the term “humanized antibody” refers to a chimeric antibody comprising amino acid residues from non-human heavy chain variable regions (HVRs) and amino acid residues from human FRs. In certain embodiments, a humanized antibody will comprise substantially all or at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

As used herein, the term “chimeric antibody” refers to an antibody in which a portion of the heavy and/or light chain is derived from a particular source or species, while the remainder of the heavy and/or light chain is derived from a different source or species.

As used herein, the term “hinge region” refers to an antibody includes the portion of a heavy chains molecule that joins the CH1 domain to the CH2 domain. This hinge region comprises approximately 25 amino acid residues and is flexible, thus allowing the two N-terminus antigen binding regions to move independently.

As used herein, the term “Fab fragments” refers to the region of the antibody structure that can bind to antigen. It consists of a complete light chain (variable and constant regions) and part of the heavy chain structure (variable and a constant region fragment), the light and heavy chains are connected by a disulfide bond. Fab fragments can be obtained by protease digestion of full-length antibodies. Under the action of papain, human immunoglobulin G can be degraded into two Fab fragments and one Fc fragment; under the action of pepsin, IgG can be degraded into an F (ab′) 2 fragment and a pFc′ fragment. The F (ab′) 2 fragment can be further reduced to form two Fab′ fragments.

As used herein, the term “Fc region” refers to a monomeric, dimeric or heterodimeric protein having at least an immunoglobulin CH2 and CH3 domain. The CH2 and CH3 domains can form at least a part of the dimeric region of the protein/molecule (e.g., antibody).

In some embodiments, the antibody means an immunoglobulin and is a molecule containing an antigen-binding site immunospecifically binding to an antigen. In some embodiments, the class of the antibody is IgG, IgE, IgM, IgD, IgA, or IgY. In some embodiments, the class of the antibody is IgG.

In some embodiments, the class of the antibody is IgG1, IgG2, IgG3 or IgG4. In some embodiments, the antibody is IgG1 or IgG4.

In some embodiments, the antibody is wild type. As use herein, the term “wild type” refers to naturally occurring and without mutation.

In some embodiments, the antibody is an engineered antibody having two amino acid substitutions of two interchain cysteines forming one interchain disulfide bond in the hinge region.

In some embodiments, the amino acid substitutions are selected from the following, cysteine to alanine, to leucine, to arginine, to lysine, to asparagines, to methionine, to aspartic acid, to phenylalanine, to praline, to glutamine, to serine, to glutamic acid, to threonine, to glycine, to tryptophan, to histidine, to tyrosine, to isoleucine or to valine, respectively.

In some embodiments, the amino acid substitutions are selected from the following, cysteine to asparagines, to glutamine, to serine, to threonine or to tyrosine, respectively.

In some embodiments, the amino acid substitutions are selected from the following, cysteine to serine.

In some embodiments, the antibody comprises at least one mutation in the Fc region. In some embodiments, the at least one mutation modulates effector function, or attenuates or eliminates Fc-g receptor binding.

In some embodiments, the one or more mutations are to stabilize the antibody and/or to increase half-life. In some instances, the one or more mutations are to modulate Fc receptor interactions, to reduce or eliminate Fc effector functions such as FcyR, antibody-dependent cell-mediated cytotoxicity (ADCC) or complement-dependent cytotoxicity (CDC). In additional instances, the one or more mutations are to modulate glycosylation.

In some embodiments, the one or more mutations are located in the Fc region. In some instances, the Fc region comprises a mutation at residue position L234, L235, or a combination thereof. In some instances, the mutations comprise L234 and L235. In some instances, the mutations comprise L234A and L235A. In some cases, the residue positions are in reference to IgG1.

In some embodiments, the Fc region comprises a mutation at residue position L234, L235, D265, N21, K46, L52, or P53, or a combination thereof. In some instances, the mutations comprise L234 and L235 in combination with a mutation at residue position K46, L52, or P53. In some cases, the residue positions are in reference to IgG1.

In some embodiments, the Fc region comprises mutations at L234, L235, and K46. In some cases, the Fc region comprises mutations at L234, L235, and L52. In some cases, the Fc region comprises mutations at L234, L235, and P53. In some cases, the Fc region comprises mutations at D265 and N21. In some cases, the residue position is in reference to IgG1.

In some instances, the Fc region comprises L234A, L235A, D265A, N21G, K46G, L52R, or P53G, or a combination thereof. In some instances, the Fc region comprises L234A and L235A in combination with K46G, L52R, or P53G. In some cases, the Fc region comprises L234A, L235A, and K46G. In some cases, the Fc region comprises L234A, L235A, and L52R. In some cases, the Fc region comprises L234A, L235A, and P53G. In some cases, the Fc region comprises D265A and N21G. In some cases, the residue position is in reference to IgG1.

In some embodiments, the Fc region comprises a mutation at residue position L233, L234, D264, N20, K45, L51, or P52. In some instances, the Fc region comprises mutations at L233 and L234 in combination with a mutation at residue position K45, L51, or P52. In some cases, the Fc region comprises mutations at L233, L234, and K45. In some cases, the Fc region comprises mutations at L233, L234, and L51. In some cases, the Fc region comprises mutations at L233, L234, and K45. In some cases, the Fc region comprises mutations at L233, L234, and P52. In some instances, the Fc region comprises mutations at D264 and N20. In some cases, equivalent positions to residue L233, L234, D264, N20, K45, L51, or P52 in an IgG1, IgG2, IgG3, or IgG4 framework are contemplated.

In some embodiments, the Fc region comprises L233A, L234A, D264A, N20G, K45G, L51R, or P52G. In some instances, the Fc region comprises L233A and L234A. In some instances, the Fc region comprises L233A and L234A in combination with K45G, L51R, or P52G. In some cases, the Fc region comprises L233A, L234A, and K45G. In some cases, the Fc region comprises L233A, L234A, and L51R. In some cases, the Fc region comprises L233A, L234A, and K45G. In some cases, the Fc region comprises L233A, L234A, and P52G. In some instances, the Fc region comprises D264A and N20G. In some cases, the residue position is in reference to IgG1.

In some embodiments, the human IgG constant region is modified to alter antibody-dependent cellular cytotoxicity (ADCC) and/or complement-dependent cytotoxicity (CDC), e.g., with an amino acid modification described inNatsume et al., 2008 Cancer Res, 68 (10): 3863-72; Idusogie et al., 2001 J Immunol, 166 (4): 2571-5; Moore et al., 2010 mAbs, 2 (2): 181-189; Lazar et al, 2006 PNAS, 103 (11): 4005-4010, Shields et al, 2001 JBC, 276 (9): 6591-6604; Stavenhagen et al., 2007 Cancer Res, 67 (18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25 (1): 1-11.

In some embodiments, the antibody of IgG1, IgG2, IgG3 or IgG4 is human or humanized antibody. The information of IgG1, IgG2, IgG3 or IgG4 can be obtained on NCBI or UniProt (https://www.uniprot.org/).

In some embodiment, the antibody is bispecific antibodies. In some embodiments of the present application, the antibody is IgG1 like bispecific antibodies.

In some embodiment, those skilled in the art can select suitable method to prepare the bispecific antibodies. In some embodiments of the present application, the bispecific antibodies can be obtained by Knobs-in-holes technology (Ridgway J B B, Presta L G, Paul C. ‘Knobs-into-holes’ engineering of antibody CH3 domains for heavy chain heterodimerization [J]. Protein Engineering (7): 617 (2023-08-11), format chain exchange (FORCE) technology, a common light chain format technology (De Nardis C, Hendriks L J A, Poirier E, et al. Anew approach for generating bispecific antibodies based on a common light chain format and the stable architecture of human immunoglobulin G1 [J]. Journal of Biological Chemistry, 2017: jbc. M117.793497), controlled Fab arm exchange technology (Yanakieva De, Pekar L, Evers A, et al. Beyond bispecificity: Controlled Fab arm exchange for the generation of antibodies with multiple specificities [J]. MABS, 2022, 14 (1), e2018960), CrossMAb technology (Klein C, Schaefer W, Regula J T. The use of CrossMAb technology for the generation of bi- and multispecific antibodies [J]. MABS, 2016, 8 (6), P1010-P1020.) or their combination.

As used herein, the term “knobs-into-holes” is used in its broadest sense and encompasses various situations, such as the CH1 domain of one heavy chain with the knob mutations and the CH1 domain of the other heavy chain with the hole mutations, the CH2 domain of one heavy chain with the knob mutations and the CH2 domain of the other heavy chain with the hole mutations, and/or the CH3 domain of one heavy chain with the knob mutations and the CH3 domain of the other heavy chain with the hole mutations. For example, and generally, “knobs-into-holes” may refer to an intra-interface modification between two antibody heavy chains in the CH3 domains: i) in the CH3 domain of one heavy chain (first CH3 domain), an amino acid residue is substituted with another amino acid residue bearing a large side chain, thereby creating a protrusion (“knob”) in the interface in the first CH3 domain; ii) in the CH3 domain of the other heavy chain (second CH3 domain), an amino acid residue is substituted with another amino acid residue bearing a smaller side chain, thereby creating a cavity (“hole”) within the interface in the second CH3 domain, in which a protrusion (“knob”) in the first CH3 domain can be placed.

In some embodiment, the antibody is selected from any one of cytotoxic antibodies, inhibitors of cell proliferation, regulators of cell activation and interaction, regulators of the human immune system, neutralizations of antigens, antibodies that are immunospectific for viral antigens or antibodies that are immunospectific for microbial antigens.

In some embodiments, the antibody can be target-specific antibodies, In some embodiments, without the limitation, the antibody can be anti-HER2 antibody, anti-FAP antibody, anti-OX-40 antibody, anti-41BB antibody, anti-Angiopoietin-2 antibody, anti-ant-IL-4Rα antibody, anti-BCMA antibody, anti-Blys antibody, anti-BTNO2 antibody, anti-C5 antibody, anti-CD122 antibody, anti-CD13 antibody, anti-CD133 antibody, anti-CD137 antibody, anti-CD138 antibody, anti-CD16a antibody, anti-CD19 antibody, anti-CD20 antibody, anti-CD22 antibody, anti-CD27 antibody, anti-CD28 antibody, anti-CD3 antibody, anti-CD30 antibody, anti-CD33 antibody, anti-CD38 antibody, anti-CD40 antibody, anti-CD47 antibody, anti-CD-8 antibody, anti-CD79 antibody, anti-CEA antibody, anti-CGPR/CGRPR antibody, anti-CSPGs antibody, anti-CTLA4 antibody, anti-CTLA-4domains antibody, anti-DLL-4 antibody, anti-EGFR antibody, anti-EpCAM antibody, anti-factor IXa antibody, anti-factor X antibody, anti-GITR antibody, anti-GP130 antibody, anti-Her3 antibody, anti-HSG antibody, anti-ICOS antibody, anti-IGF1 antibody, anti-IGF1/2 antibody, anti-IGF-1R antibody, anti-IGF2 antibody, anti-IGFR antibody, anti-IL-1 antibody, anti-IL-12 antibody, anti-IL-12p40 antibody, anti-IL-13 antibody, anti-IL-17A antibody, anti-IL-1β antibody, anti-IL-23 antibody, anti-IL-5 antibody, anti-IL-6 antibody, anti-IL-6R antibody, anti-Lag-3 antibody, anti-LAG3 antibody, anti-MAG antibody, anti-Met antibody, anti-NgR antibody, anti-NogoA antibody, anti-OMGp antibody, anti-OX40 antibody, anti-PD-1 antibody, anti-PDGFR antibody, anti-PDL-1 antibody, anti-PSMA antibody, anti-RGMA antibody, anti-RGMB antibody, anti-SARS-CoV-2 antibody, anti-Te38 antibody, anti-TIM-3 antibody, anti-TNF antibody, anti-TNFα antibody, anti-TROP-2 antibody, anti-TWEAK antibody, anti-VEGF antibody, or anti-VEGFR antibody.

In some embodiments, the antibody is target-specific, which is targeted to, HER2 (Human Epidermal GrowthFactor Receptor 2), TROP2 (TACSTD2, tumor associated calcium signal transducer 2), BCMA (TNFRSFi7, TNF receptor superfamily member 17).

In some embodiments, the antibody can be Transtuzumab, Sacituzumab, Belantamab, Risankizumab, Eptinezumab, Teprotumumab, Polatuzumab, Tafasitamab, Rovelizumab, Romosozumab, Dostarlimab, Enfortumab or Ublituximab. In some embodiments, the antibody is Trastuzumab, Sacituzumab or Belantamab.

In some embodiments, the antibody can be obtained commercially or produced by any method known to those skilled in the art.

In some embodiments, the first thiobridge reagent and the second thiobridge reagent independently contain at least two substituted groups allowing a re-bridging of the thiol groups.

In some embodiments, without the limitation, the first thiobridge reagent and the second thiobridge reagent are independently selected from the group consisting of

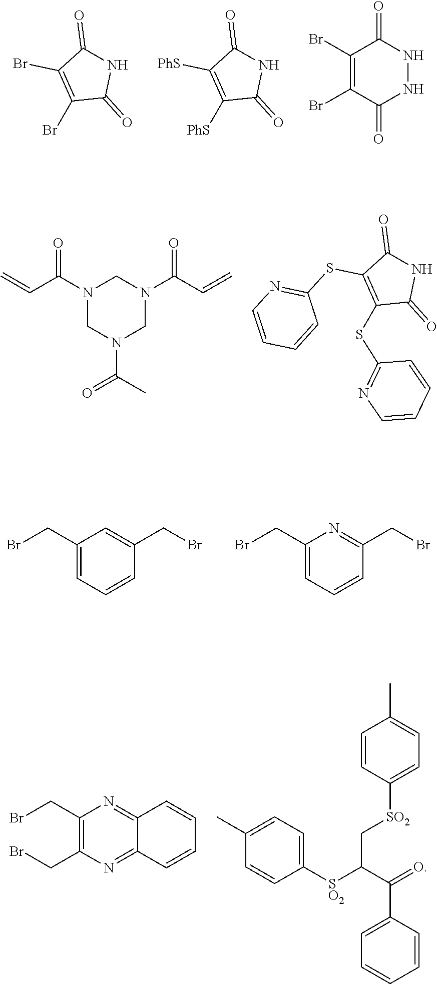

In some embodiments, the reactive groups independently contain azido and/or dibenzocyclooctyne (DBCO).

In some embodiments, the thiobridge reagent and the reactive groups are connected by alkyl group or polyethylene glycol (PEG).

In some embodiments, without the limitation, the first thiobridge reagent bearing reactive groups and the second thiobridge reagent bearing reactive groups are independently selected from the groups consisting of

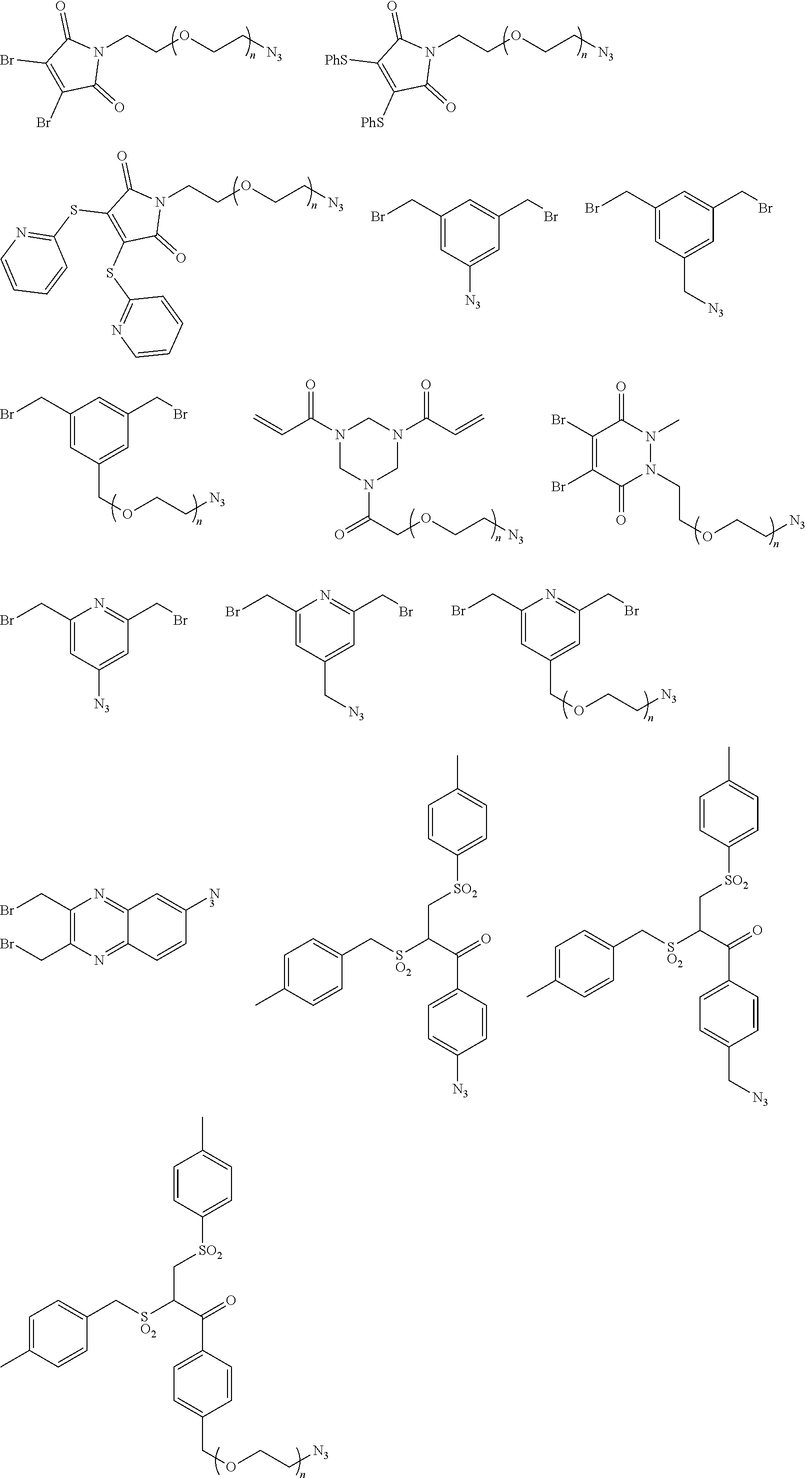

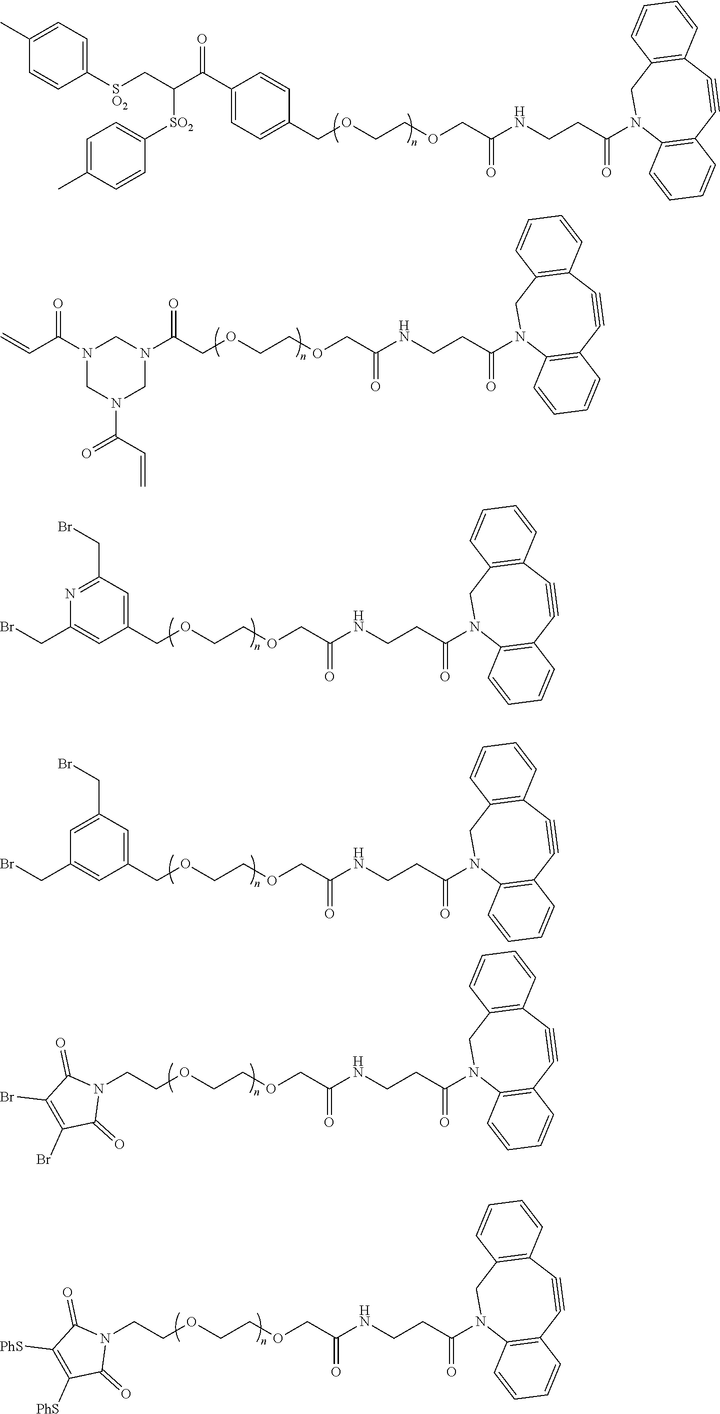

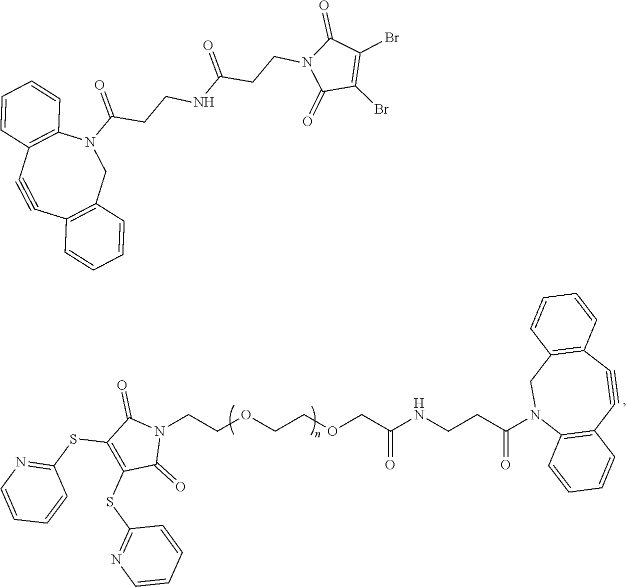

wherein, n is 0-20, 0-18, 0-15, 0-13, 0-10, 0-7, 0-5 or 0-3, optionally, n is 0, 1, 2, 3, 4, 5, 6, 7, 8, 9 or 10.

In some embodiments, the first thiobridge reagent bearing reactive groups and the second thiobridge reagent bearing reactive groups are dibromomaleimide-PEG4-N3, having the following formula

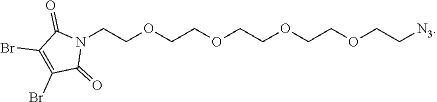 [SMILES: [N-]=[N+]=NCCOCCOCCOCCOCCN1C(=O)C(Br)=C(Br)C1=O]

In some embodiments, a linker of the first linker-payload and the second linker payload is selected from any one of which the one terminal can be connected to reduced thiol group of the antibody or the reactive groups of the thiobridge reagent, and the other terminal can be connected to the payload.

As used herein, the term “linker” refers to a substituted molecule which contains at least two subsituted groups, one of which can covalently bond to a drug molecule and the other of which can covalently couple to an antibody or the reactive groups of the thiobridge reagent.

In some embodiments, when the first linker-payload and/or the second linker-payload react (s) with the reduced thiol groups, the linker of the first linker-payload and the second linker-payload independently includes a cleavable linker or a noncleavable linker. Cleavable linkers can be chemically labile and enzyme-labile linkers. Due to the high plasma stability and good intracellular cleaving selectivity and efficiency, enzyme-labile linkers are broadly selected as cleavable linker candidates in ADCs. In some embodiments, enzyme-labile linkers comprise the structure: -maleimidocaproyl- (-MC-), -maleimidocaproyl-peptide moiety- (-MC-peptide moiety-), -p-aminobenzyl alcohol- (-PAB-), or -peptide moiety-. In some embodiments, the peptide moiety is dipeptides, tripeptides, tetrapeptides or pentapeptides.

In some embodiments, without the limitation, the dipeptides can be valine-alanine (VA), valine-citrulline (VC), alanine-asparagine (AD), alanine-phenylalanine (AF), phenylalanine-lysine (FK), alanine-lysine (AK), alanine-valine (AV), valine-lysine (VK), lysine-lysine (KK), phenylalanine-citrulline (FC), leucine-citrulline (LC), isoleucine-citrulline (IC), tryptophan-citrulline (WC) or phenylalanine-alanine (FA).

In some embodiments, without the limitation, the tripeptides can be alanine-alanine-asparagine (AAD), glycine-valine-citrulline (GVC), glycine-glycine-glycine (GGG), phenylalanine-phenylalanine-lysine (FFK), glutamic acid-valine-citrulline (EVC), or glycine-phenylalanine-lysine (GFK).

In some embodiments, without the limitation, the tetrapeptides can be glycine-glycine-phenylalanine-glycine (GGFG).

In some embodiments, without the limitation, when the first linker-payload and/or the second linker-payload react (s) with the reduced thiol groups, the linker of the first linker-payload and the second linker-payload can be MC-VA-PAB, MC-VC-PAB, MC-AD-PAB, MC-AF-PAB, MC-FK-PAB, MC-AK-PAB, MC-AV-PAB, MC-VK—PAB, MC-KK-PAB, MC-FC-PAB, MC-LC-PAB, MC-IC-PAB, MC-WC-PAB or MC-FA-PAB independently. In some embodiments, without the limitation, when the first linker-payload and/or the second linker-payload react (s) with the reduced thiol groups, the linker of the first linker-payload and the second linker-payload can be MC-AAD-PAB, MC-GVC-PAB, MC-GGG-PAB, MC-FFK-PAB, MC-EVC-PAB, or MC-GFK-PAB independently.

In some embodiments, the linker comprises a maleimide bearing a drug, an organic chloride bearing a drug, an organic bromide bearing a drug, an organic iodide bearing a drug and/or vinylpyrimidine bearing a drug.

In some embodiments, when the first linker-payload and/or the second linker-payload react (s) with the reactive groups in the thiobridge reagent, the linker of the first linker-payload and/or the second linker-payload further include (s) azido and/or dibenzocyclooctyne (DBCO). In some embodiments, when the linker of the first linker-payload and/or the second linker-payload contains azido, the reactive groups of the thiobridge group contain DBCO. In some embodiments, when the linker of the first linker-payload and/or the second linker-payload contains DBCO, the reactive groups of the thiobridge group contain azido.

In some embodiments, when the first linker-payload and/or the second linker-payload react (s) with the reactive groups in the thiobridge reagent, the linker of the first linker-payload and the second linker-payload is independently selected from any one of the groups consisting of

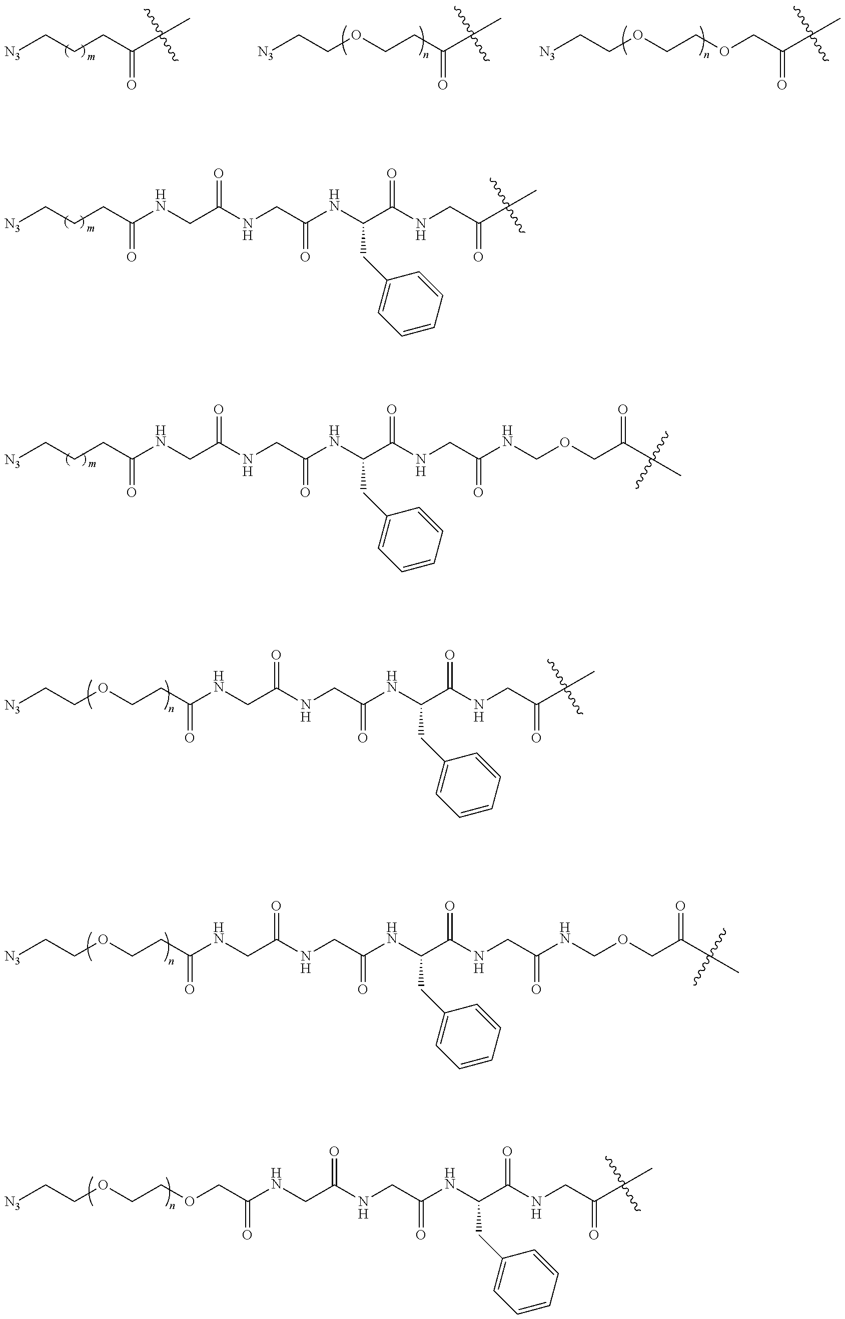

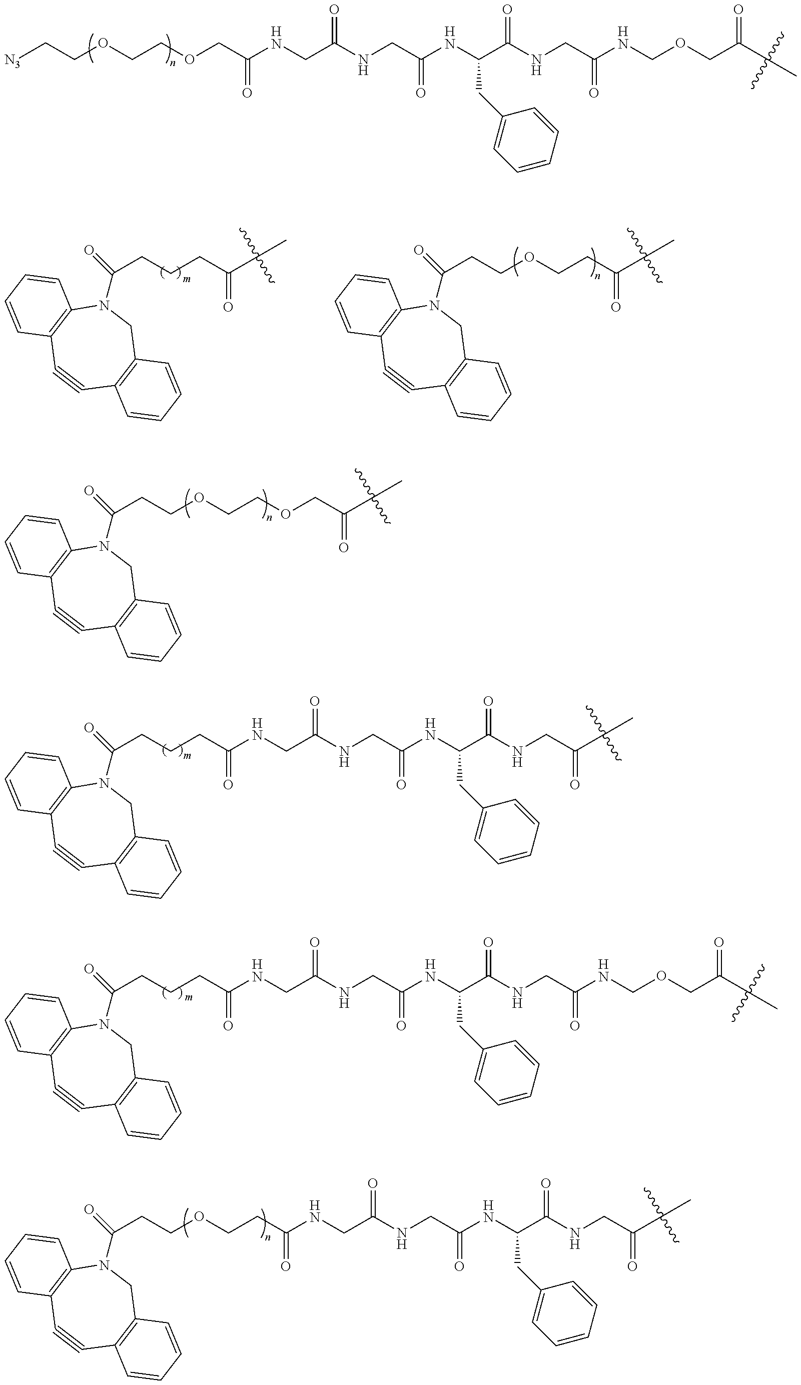

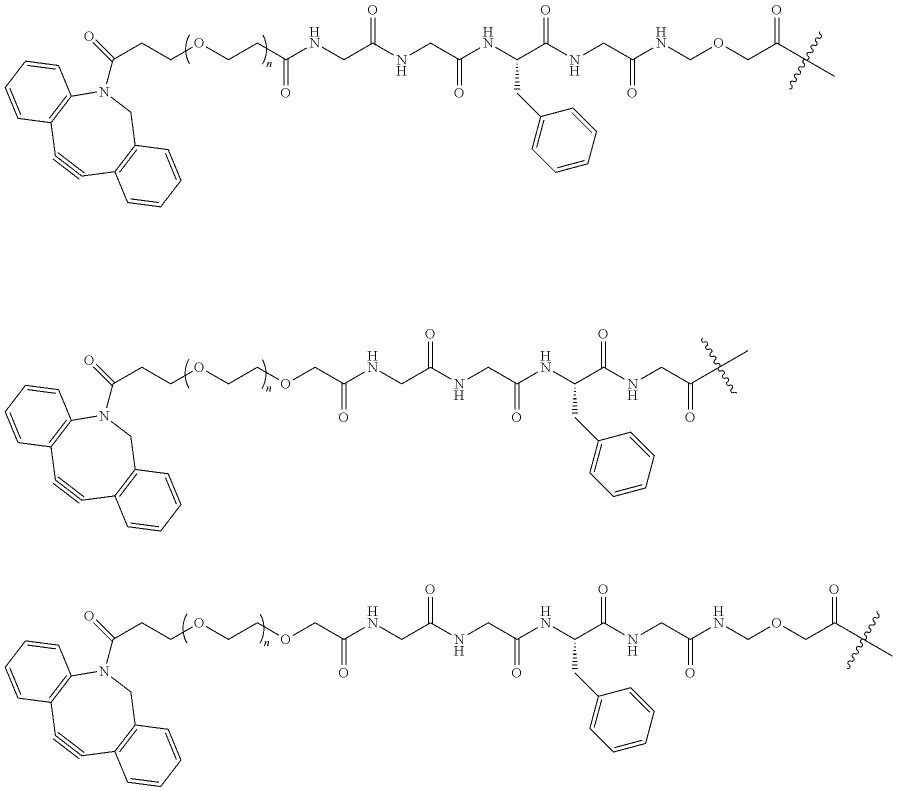

wherein, n is 0-20, 0-18, 0-15, 0-13, 0-10, 0-7, 0-5 or 0-3, m is 0-20, 0-18, 0-15, 0-13, 0-10, 0-7, 0-5 or 0-3, optionally, n is 0, 1, 2, 3, 4, 5, 6, 7, 8, 9 or 10, m is 0, 1, 2, 3, 4, 5, 6, 7, 8, 9 or 10.

As used herein, the term “end capping reagent” refers to a compound which does not bear a drug and contains at least one substituted group which can covalently couple to an antibody.

In some embodiments, the end capping reagent is the cleavable linker or the noncleavable linker. In some embodiments, the end capping reagent is (2-Aminoethyl) maleimide.

In some embodiments, there is no specific limitation to the payload, as long as the payload contains at least one substituted group allowing a connection from the payload to the linker.

As used herein, the term “payload” refers to any cytotoxic molecule at least one substituted group or a partial structure allowing connection to the linker structure. The payload may kill cancer cells and/or inhibit growth, proliferation, or metastasis of cancer cells, thereby reducing, alleviating, or eliminating one or more symptoms of a disease or disorder.

In some embodiments, the payload is a cytotoxic drug, a cytokine, a nucleic acid, a radionuclide, a kinase or derivatives thereof. In some embodiments, the payload includes but not limited to topoisomerases inhibitor and tubulin inhibitors. In some embodiments, without the limitation, the payload can be anti-cancer agent, antiviral agent or antimicrobial agent.

In some embodiments, the cancer is carcinoma, lymphoma, blastema, sarcoma, and leukemia or lymphoid malignancies. More particular examples of the cancer include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Exemplary payloads are monomethyl auristatin E (MMAE), monomethyl auristatin D (MMAD), monomethyl auristatin EF (MMAF), calicheamicins (CLM), mertansine (DM1), maytansinoids, duocarmycins, anthracyclines, pyrrolobenzodiazepine dimers, amatoxin, quinolinealkaloid, Dxd, doxorubicin hydrochloride, methotrexate, erlotinib, bortezomib, fulvestrant, sunitib imatinib mesylate, letrozole, finasunate, platins such as oxaliplatin, carboplatin, and cisplatin, finasunate, fluorouracil, rapamycin, leucovorin, lapatinib, lonafamib, sorafenib, gefitinib, capmtothecin, topotecan, bryostatin, adezelesin, anthracyclin, carzelesin, bizelesin, dolastatin, auristatins, duocarmycin, eleutherobin, taxols such as paclitaxel or docetaxel, cyclophasphamide, doxorubicin, vincristine, prednisone or prednisolone, other alkylating agents such as mechlorethamine, chlorambucil, and ifosfamide, antimetabolites such as azathioprine or mercaptopurine, vincristine, vinblastine, vinorelbine, vindesine, etoposide, teniposide, etoposide phosphate, epipodophyllotoxins, actinomycin, daunorubicin, valrubicin, idarubicin, edrecolomab, epirubicin bleomycin, plicamycin or mitomycin, and salts thereof.

In some embodiments, the payload is deruxtecan (DXd), cyanine 3 (Cy3), MMAE, MMAD or MMAF. In some embodiments of the present application, the payload is MMAE, DXd or Cy3.

The linker-payload is a chemical moiety, which is synthesized by connecting a linker to a payload. Depending on the desired payload and selected linker, those skilled in the art can select suitable method for coupling them together. For example, some conventional coupling methods, such as amine coupling methods, may be used to form the desired linker-payload which still contains reactive groups for conjugating to the antibodies through covalent linkage. A drug-maleimide complex (i.e., maleimide linking drug) is taken as an example of the payload bearing reactive group in the present disclosure. Most common reactive group capable of bonding to thiol group in ADC preparation is maleimide. Additionally, organic chloride, bromides, iodides also are frequently used.

The linker-payload could be any physical active compound, or any compound used to diagnose, prevent or treat a disease. In some embodiments, when the first linker-payload and/or the second linker-payload react (s) with the reduced thiol groups, the first linker-payload and/or the second linker-payload are independently MC-VC-PAB-MMAE, MC-VC-PAB-MMAD and MC-VC-PAB-MMAF.

In some embodiments, the first thiobridge reagent bearing the first linker-payload and the second thiobridge reagent bearing the second linker-payload independently have the following formula: Q-S-T,
wherein, Q is selected from the groups consisting of

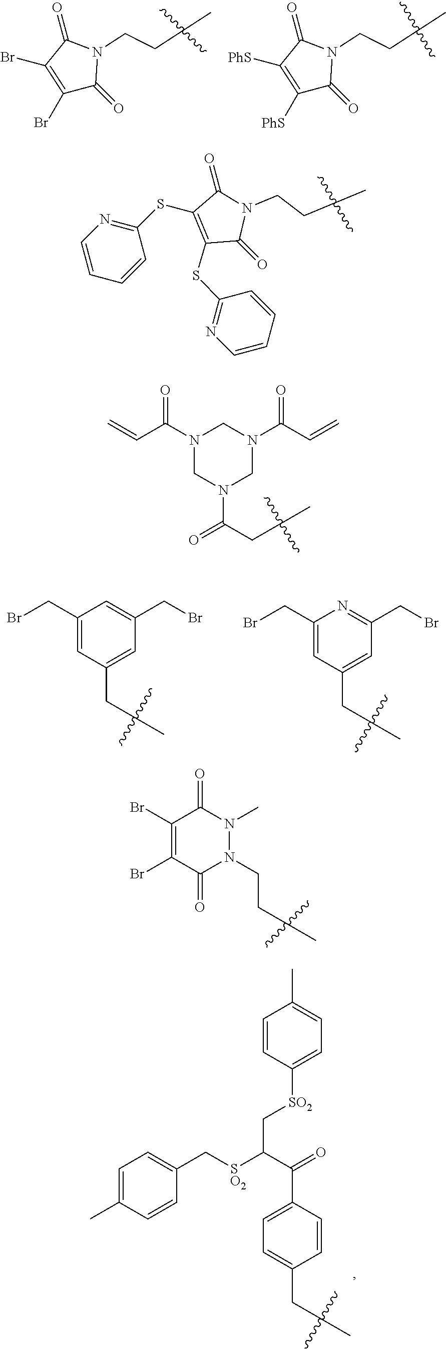

S is selected from a cleavable linker or a non-cleavable linker, without the limitation, S is selected from the groups consisting of

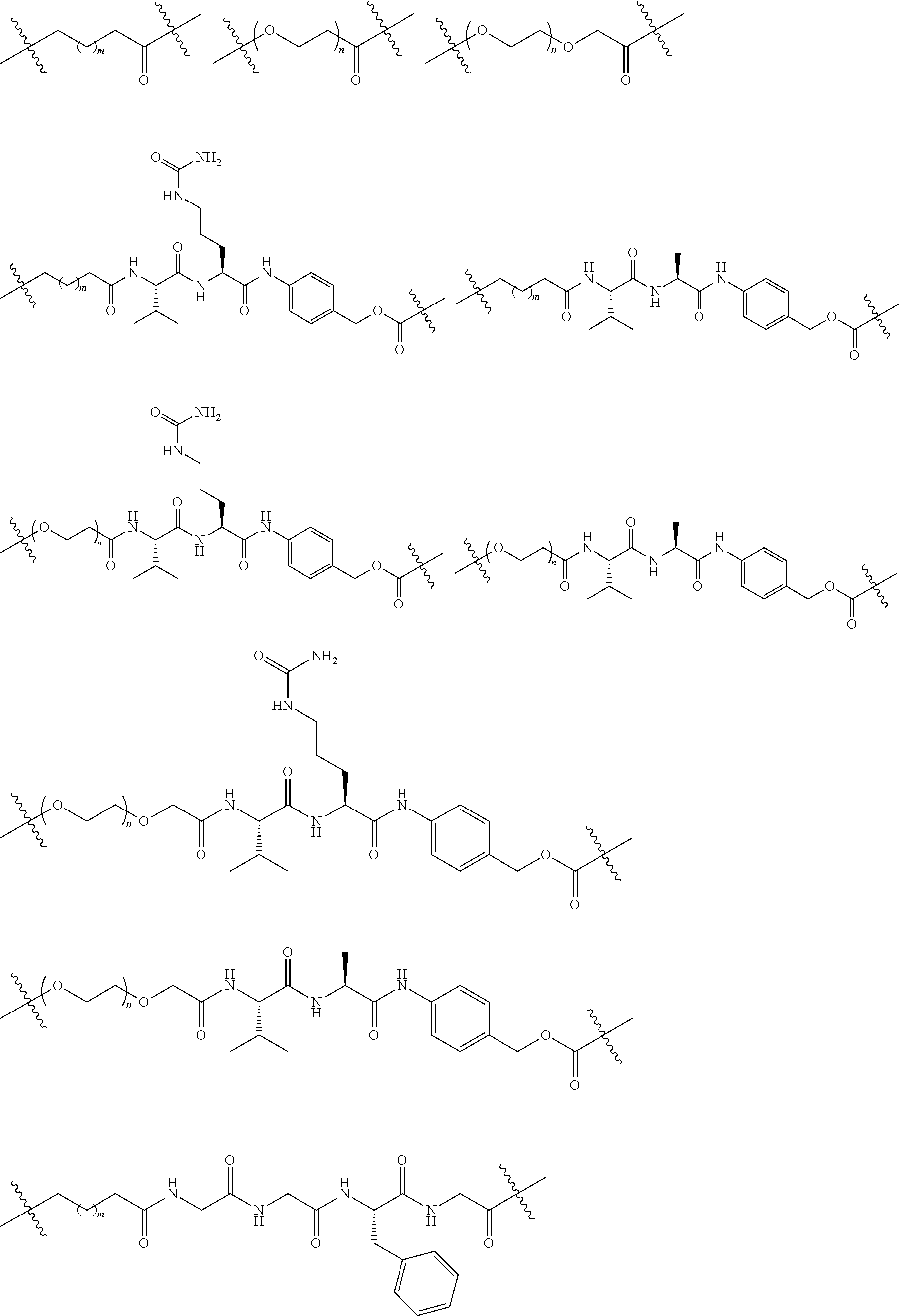

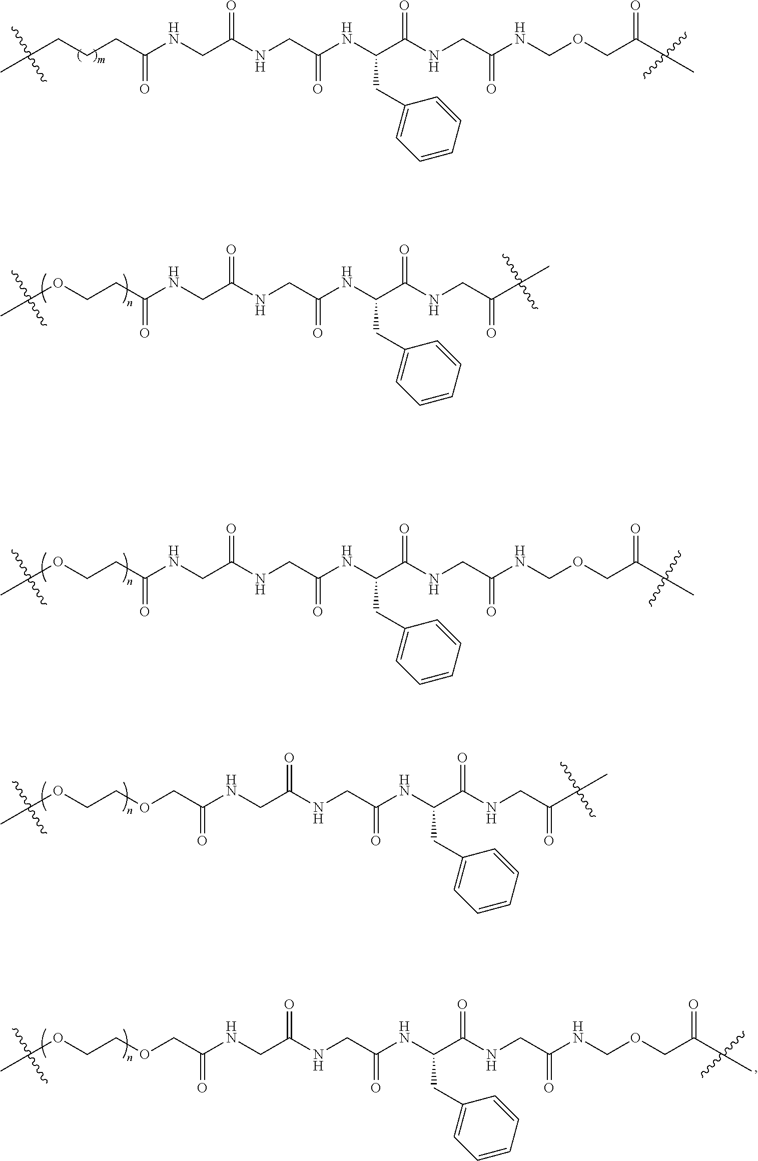

Wherein, n is 0-20, m is 0-20, optionally, n is 0, 1, 2, 3, 4, 5, 6, 7, 8, 9 or 10, m is 0, 1, 2, 3, 4, 5, 6, 7, 8, 9 or 10,

T is payload.

In some embodiments, without the limitation, the first thiobridge reagent bearing the first linker-payload and the second thiobridge reagent bearing the second linker-payload are independently selected from the group consisting of

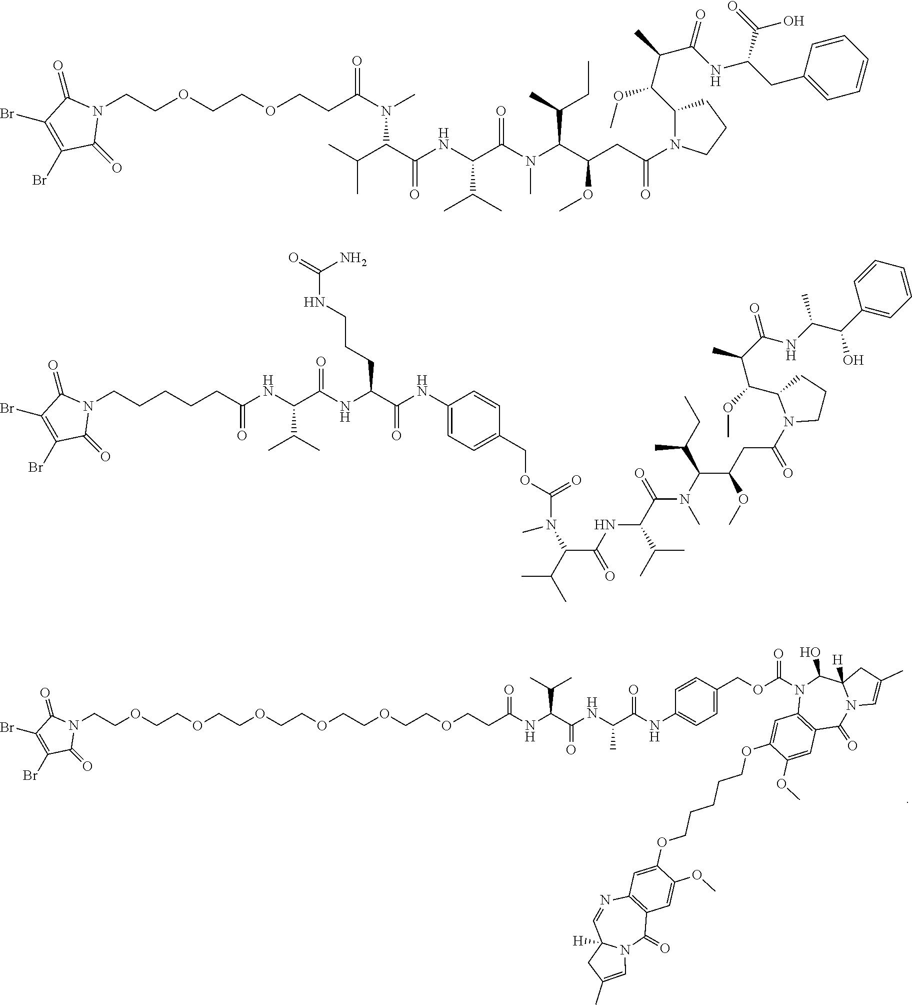

In some embodiments, the payload of the first thiobridge reagent bearing the first linker-payload and that of the second thiobridge reagent bearing the second linker-payload are different or same. In some embodiments, the linker of the first thiobridge reagent bearing the first linker-payload and that of the second thiobridge reagent bearing the second linker-payload could be different or same. In some embodiments, the thiobridge reagent of the first thiobridge reagent bearing the first linker-payload and that of the second thiobridge reagent bearing the second linker-payload could be different or same.

In some embodiments, said method of preparing the ADC with D2 comprises the following steps:
(a1) incubating the compound according to the present application which works as the first reductant and the antibody in the presence of an effective amount of the transition metal ions in the first buffer system to selectively reduce the interchain disulfide bonds with the antibody; or
incubating the composition according to the present application, in which the compound according to the present application works as the first reductant, and the antibody in the first buffer system to selectively reduce the interchain disulfide bonds within the antibody;
(b1) introducing an excess amount of the metal chelators and an excess amount of the first linker-payload to react with the reduced thiol groups resulted from step (a1).

In some embodiments, the reaction temperature is 15° C. to 25° C. in step (b1), the reaction time is 0.5 h to 2 h in step (b1).

In some embodiments, the homogeneity of the ADC with D2 is up to 53%, 55%, 60%, 65%, 70%, 75%, 80%, 84%, 87%, 89%, 90%, 91%, 92% or 94%.

In some embodiments, the ADC prepared by the method preparing the ADC with D2 comprises D0 and D4 in a content less than 50%, 40%, 35%, 30%, 25%, 23%, 22% or 21% of the total weight of D0, D2, D4, D6 and D8. In some embodiments, the ADC prepared by the method preparing the ADC with D2 comprises D0 and D4 in a content less than 20% of the total weight of D0, D2, D4, D6 and D8.

In some embodiments, the method of preparing the ADC with D2+D6 comprises the following steps:
(c2) incubating the reaction product from step (b1) and the second reductant in the second buffer system to reduce the interchain disulfide bonds within the reaction product from step (b1);
(d2) introducing the incubation product from step (c2) and an excess amount of the second linker-payload to react with the reduced thiol groups resulted from step (c2).

In some embodiments, the homogeneity of the ADC with D2+D6 is up to 75%, 80%, 85%, even to 90%.

In some embodiments, the method of preparing the ADC with D2+D3 comprises the following steps:
(d3) introducing the incubation product from step (c2) and an excess amount of the second thiobridge reagent bearing the second linker-payload to react with the reduced thiol groups resulted from the (c2).

In some embodiments, the method of preparing the ADC with D2+D3 comprises the following the steps:
(d3′) introducing the incubation product from step (c2) and an excess amount of the second thiobridge reagent bearing reactive groups to re-bridge the reduced thiol groups resulted from step (c2), then, incubating an excess amount of the second linker-payload in the second buffer system to react with the reactive groups of the thiobridge group.

In some embodiments, the method of preparing the ADC with D1 comprises the following steps:
(b4) introducing an excess amount of the metal chelators and an excess amount of the first thiobridge reagent bearing the first linker-payload to react with the reduced thiol groups resulted from step (a1).

In some embodiments, the method of preparing the ADC with D1 comprises the following the steps:
(b4′) introducing an excess amount of the metal chelators and the first thiobridge reagent bearing reactive groups to re-bridge the reduced thiol groups resulted from step (a1), then, incubating an excess amount of the first linker-payload in the first buffer system to react with the reactive groups of the thiobridge group.

In some embodiments, the homogeneity of the ADC with D1 is up to 75%, 80%, 85%, even to 90%.

In some embodiments, the method of preparing the ADC with D1+D6 comprises the following steps:
(c5) incubating the reaction product from step (b4) or step (b4′) and the second reductant in the second buffer system to reduce the interchain disulfide bonds within the reaction product from step (b4) or (b4′);
(d5) introducing the incubation product from step (c5) and an excess amount of the second linker-payload to react with the reduced thiol groups resulted from step (c5).

In some embodiments, the method of preparing the ADC with D1+D3 comprises the following steps:
(d6) introducing the incubation product from step (c5) and an excess amount of the second thiobridge reagent bearing the second linker-payload to react with the reduced thiol groups resulted from the (c5).

In some embodiments, the method of preparing the ADC with D1+D3 comprises the following the steps:
(d6′) introducing the incubation product from step (c5) and an excess amount of the second thiobridge reagent bearing reactive groups to re-bridge the reduced thiol groups resulted from step (c5), then, incubating an excess amount of the second linker-payload in the second buffer system to react with the reactive groups of the thiobridge group.

In some embodiments, the method of preparing the ADC with D0+D6 comprises the following steps:
(b7) introducing an excess amount of the metal chelators and an excess amount of the first thiobridge reagent to react with the reduced thiol groups resulted from step (a1).
(c7) incubating the reaction product from step (b7) and the second reductant in the second buffer system to reduce the interchain disulfide bonds within the reaction product from step (b7):
(d7) introducing the incubation product from step (c7) and an excess amount of the second linker-payload to react with the reduced thiol groups resulted from step (c7).

In some embodiments, the homogeneity of the ADC with D0+D6 is up to 75%, 80%, 85%, even to 90%.

In some embodiments, the method of preparing the ADC with D0+D3 comprises the following steps:
(d8) introducing the incubation product from step (c7) and an excess amount of the second thiobridge reagent bearing the second linker-payload to react with the reduced thiol groups resulted from the (c7).

In some embodiments, the method of preparing the ADC with D0+D3 comprises the following the steps:
(d8′) introducing the incubation product from step (c7) and an excess amount of the second thiobridge reagent bearing reactive groups to re-bridge the reduced thiol groups resulted from step (c7), then, incubating an excess amount of the second linker-payload in the second buffer system to react with the reactive groups of the thiobridge group.

In some embodiments, the antibody with site-specific modification (ADC with D1+D4, ADC with D1+D2) prepared by the method including the step (a), (b), (c) and (d), wherein the modification reagent 1 is the first thiobridge reagent bearing the first linker-payloads, and the modification reagent 2 is the second linker-payloads. Meanwhile, the transition metal ions are introduced in step (c).

In some embodiments, the antibody with site-specific modification (ADC with D1+D4, ADC with D1+D2) prepared by the method including the step (a), (b), (c) and (d), wherein the modification reagent 1 is the first thiobridge reagent bearing reactive groups which reacts with the first linker-payloads, and the modification reagent 2 is the second linker-payloads. Meanwhile, the transition metal ions are introduced in step (c).

In some embodiments, the antibody with site-specific modification (ADC with D2+D4, ADC with D2+D2) prepared by the method including the step (a), (b), (c) and (d), wherein the modification reagent 1 is the first linker-payloads, and the modification reagent 2 is the second linker-payloads. Meanwhile, the transition metal ions are introduced in step (c).

In some embodiments, the antibody with site-specific modification (ADC with D0+D4) prepared by the method including the step (a), (b), (c) and (d), wherein the modification reagent 1 is the first thiobridge reagent or the end capping reagents, and the modification reagent 2 is the second linker-payloads. Meanwhile, the transition metal ions are introduced in step (c).

Various analytical methods can be used to determine the yields and isomeric mixtures of the ADC. In some embodiments, the analytical method is HIC-HPLC. HIC-HPLC is able to separate the ADC which antibodies loaded with various numbers of drugs. The drug loading level can be determined based on the ratio of absorbances, e.g., at 250 nm and 280 nm. For example, if a drug can absorb at 250 nm while the antibody absorbs at 280 nm. The 250/280 ratio therefore increases with drug loading.

As compared with ADCs generated by conventional conjugation processes, using the bio-conjugation process described herein, the ADCs of the present application have improved homogeneity without need of protein engineering, without need of ligases, and has simple manipulation and reduced cost.

The process of generating ADC with homogeneous D2 by selectively reducing one of four interchain disulfide bonds on IgG antibodies bypasses any need of protein engineering or enzyme catalysis, but is based on native inter-chain disulfide bonds, and only needs novel reductants and transition metal ions. Therefore, as compared with conventional processes for preparing ADC, the process of the disclosure is less complicate, the homogeneity of the resultant antibody-drug conjugate is dramatically improved.

In some embodiments, the method of preparing the ADC with D1 comprises the following steps:
(A1) incubating the first reductant (0.02 mM) and trastuzumab (0.012 mM) in the presence of an effective amount of ZnCl₂ (0.24 mM) in MES (20 mM, pH6.7), The incubation temperature is 4° cand the incubation time is 4 h;
(B1) introducing EDTA (0.6 mM) and an excess amount of dibromomaleimide-PEG4-N3 (0.013 mM) to react with reduced thiol groups resulted from step (A1), the reaction temperature is 24° C. and the reaction time is 3 h, then recovering the product using a desalting column;
(C1) incubating the product form step (B1) and DBCO-Cy3 (0.02 mM) in MES (20 mM, pH6.7), the reaction temperature is 25° C. and the reaction time is 8 h, then recovering the resultant ADC with D1 using a desalting column.

In some embodiments, the method of preparing the ADC with D0+D6 comprises the following steps:
(A2) incubating the first reductant (0.02 mM) and trastuzumab (0.012 mM) in the presence of an effective amount of ZnCl₂ (0.24 mM) in MES (20 mM, pH6.7), The incubation temperature is 4° C. and the incubation time is 4 h;
(B2) introducing EDTA (0.6 mM) and an excess amount of dibromomaleimide (0.013 mM) to react with reduced thiol groups resulted from step (A2), the reaction temperature is 24° C. and the reaction time is 3 h, then recovering the product using a desalting column;
(C2) incubating the product form step (B2) and TCEP (0.08 mM) in MES (20 mM, pH6.7), the reaction temperature is 25° C. and the reaction time is 12 h;
(D2) introducing the second linker-payload (MC-GGFG-DXd, 0.14 mM) to step (C2), and the reaction mixture was allowed to stay at 24° C. for 1 h, then recovering the resultant ADC with D0+D6 using a desalting column.

In some embodiment, the method of preparing the ADC with D2+D6 comprises the following steps:
(A3) incubating the first reductant (0.02 mM) and trastuzumab (0.012 mM) in the presence of an effective amount of ZnCl₂ (0.24 mM) in MES (20 mM, pH6.7), The incubation temperature is 4° C. and the incubation time is 4 h;
(B3) introducing EDTA (0.6 mM) and an excess amount of MC-MMAF (0.06 mM) to react with reduced thiol groups resulted from step (A3), the reaction temperature is 24° C. and the reaction time is 1 h, then recovering the product using a desalting column;
(C3) incubating the product form step (B3) and TCEP (0.08 mM) in MES (20 mM, pH6.7), the reaction temperature is 25° C. and the reaction time is 8 h;
(D2) introducing the second linker-payload (MC-GGFG-DXd, 0.14 mM) to step (C3), and the reaction mixture was allowed to stay at 24° C. for 1 h, then recovering the resultant bi-payload ADC with D2+D6 using a desalting column.

In some embodiments, the method of preparing the ADC with D2 comprises the following steps:
(A4) Incubating the first reductant (0.02 mM) and Transtuzumab (0.012 mM) in the presence of ZnCl₂ (0.24 mM) in MES buffer (pH6.7, 20 mM). The incubation temperature is 4° C. and the incubation time is 4 h;
(B4) EDTA (0.6 mM) was added to trap Zn²⁺;
(C4) Introducing MC-VC-PAB-MMAE (0.06 mM) to react with reduced thiol groups resulted from step (1), the reaction temperature is 24° C. and the reaction time is 1 h;
(D4) Introducing cysteine (0.08 mM) to consume excessive MC-VC-PAB-MMAE;
(E4) The resulted ADC was subjected to purification using a de-salting column.
   An Antibody with Thiol Group Site-Specific Modifications

The present application provides an antibody with thiol group site-specific modifications prepared by the method of the present application.

In some embodiments, the antibody with thiol group site-specific modifications is conjugated with the modification reagent 1 and/or the modification reagent 2.

In some embodiments, the modification reagent 1 and/or the modification 2 are covalently linked to the reduced thiol groups in the hinge region of the antibody.

In some embodiments, the modification reagent 1 and/or the modification 2 are covalently linked to the reduced thiol groups in the Fab region of the antibody.

In some embodiments, the antibody with thiol group site-specific modifications is conjugated with the modification reagent 1, forming the ADC with D2 or the ADC with D1. In some embodiments of the present application, the antibody with thiol group site-specific modifications is conjugated with the modification reagent 1 and the modification reagent 2, forming the ADC with D2+D6, the ADC with D2+D3, the ADC with D1+D6, the ADC with D1+D3, the ADC with D0+D6, the ADC with D0+D3, the ADC with D0+D4, the ADC with D2+D4, the ADC with D1+D4, the ADC with D2+D2 or the ADC with D1+D2.

In some embodiments, the ADC with D2 is Trastuzumab-[MC-VC-PAB-MMAE]₂, Sacituzumab-[MC-VC-PAB-MMAE]₂ or Belantamab-[MC-VC-PAB-MMAE]₂.

In some embodiments, the ADC with D1 is Trastuzumab-[Maleimide-PEG4-N3-DBCO-Cy3]1.

In some embodiments, the ADC with D0+D6 is Trastuzumab-[Maleimide]₁ [MC-GGFG-DXd]₆.

In some embodiments, the ADC with D2+D6 is Trastuzumab-[MC-MMAF]₂ [MC-GGFG-DXd]₆.

In some embodiments, the ADCs comprise Trastuzumab-[MC-VC-PAB-MMAE]₂ [MC-GGFG-DXd]₂, Trastuzumab-[Maleimide-PEG4-N3-DBCO-Cy3]₁ [MC-VC-PAB-MMAE]₂, Trastuzumab-[Maleimide]₁ [MC-VC-PAB-MMAE]4, Trastuzumab-[MC-GGFG-DXd]₂ [MC-VC-PAB-MMAE]4 or Trastuzumab-[Maleimide-PEG4-N3-DBCO-Cy3]₁ [MC-VC-PAB-MMAE]₄.

Use of the Antibody with Thiol Group Site-Specific Modifications

The disclosure provides the use of the antibody with thiol group site-specific modifications according to the present application in the manufacture of a therapeutic agent for preventing, diagnosing or treating a disease.

As use herein, the term “treat” of any disease refers to alleviating or ameliorating the disease (i.e., slowing or arresting the development of the disease or at least one of the clinical symptoms thereof); or alleviating or ameliorating at least one physical parameter or biomarker associated with the disease, including those which may not be discernible to the patient. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, delaying the development of a tumor, or some combination thereof.

As used herein, the term “prevent” of any disease refers to the prophylactic treatment of the disease; or delaying the onset or progression of the disease.

In some embodiments, the disease is a tumor or cancer. In some embodiments, the disease is an autoimmune disease and the like.

In some embodiments, the cancer can include, but not limited to, carcinoma, lymphoma, blastema, sarcoma, and leukemia or lymphoid malignancies. More particular examples of the cancer include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

### Pharmaceutical Composition

The present application also provides a pharmaceutical composition comprising the antibody with thiol group site-specific modifications prepared by the method described above and at least a pharmaceutically acceptable carrier.

Pharmaceutical compositions provided herein may be formulated in any manner known in the art, such as, pharmaceutical compositions provided herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions are formulated to be compatible with their intended route of administration (e.g., intravenous, intraarterial, intramuscular, intradermal, subcutaneous, or intraperitoneal).

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxyanisole, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine.

In some embodiments, the pharmaceutical compositions can be a liquid solution, suspension, or emulsion. In some embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In some embodiments, the pharmaceutical composition is combined with other therapeutic agents. There is no specific limitation to the other therapeutic agents, as long as the other therapeutic agents can reduce the side effects of the pharmaceutical composition or increase the efficacy of the pharmaceutical composition. The other therapeutic agents are anti-cancer agents, anti-autoimmune disease agent, anti-emetics, anti-allergic and the like.

In some embodiments, the anti-cancer agents can include, but not limited to, erlotinib, bortezomib, fulvestrant, sunitib imatinib, mesylate, letrozole, finasunate, platins such as oxaliplatin, carboplatin, and cisplatin, finasunate, fluorouracil, rapamycin, leucovorin, lapatinib, lonafamib, sorafenib, gefitinib, capmtothecin, topotecan, bryostatin, adezelesin, anthracyclin, carzelesin, bizelesin, dolastatin, auristatins, duocarmycin, eleutherobin, taxols such as paclitaxel or docetaxel, cyclophasphamide, doxorubicin, vincristine, prednisone or prednisolone, other alkylating agents such as mechlorethamine, chlorambucil, and ifosfamide, antimetabolites such as azathioprine or mercaptopurine, other microtubule inhibitors (vinca alkaloids like vincristine, vinblastine, vinorelbine and vindesine, as well as taxanes), podophyllotoxins (etoposide, teniposide, etoposide phosphate, and epipodophyllotoxins), topoisomerase inhibitors, other cytotoxins such as actinomycin, daunorubicin, valrubicin, idarubicin, epirubicin, bleomycin, plicamycin, mitomycin and the like.

In some embodiments, the anti-autoimmune disease agent can include, but not limited to, ibuprofen, loxoprofen, naproxen, diclofenac, indomethacin, meloxicam, lornoxicam, nabumetone, celecoxib, paracetamol, glucocorticoids, azathioprine, cyclophosphamide and the like.

In some embodiments, patients may experience nausea during and after administration of the ADCs of the present application. Therefore, anti-emetics may be administered in preventing nausea (upper stomach) and vomiting. The anti-emetics can include, but not limited to, aprepitant, ondansetron, granisetron HCl, lorazepam, dexamethasone, prochlorperazine, casopitant and the like.

In some embodiments, patients may experience allergic reactions during and after administration of the ADCs of the present application. Therefore, anti-allergic agents may be administered to minimize the risk of an allergic reaction. The anti-allergic agents include dexamethasone, beclomethasone, hydrocortisone, prednisolone, prednisone, methylprednisolone, hydroxyzine, cyproheptadine, bronchodilators, terbutaline and the like.

The disclosure provides the method of preventing, diagnosing or treating a disease in a subject in need thereof, comprising administrating to the subject a therapeutically effective amount of the antibody with thiol group site-specific modifications prepared by the method described above or the pharmaceutical composition according to the present application.

As use herein, the term “subject” refers to mammals, primates (e.g., humans, male or female), dogs, rabbits, guinea pigs, pigs, rats and mice. In certain embodiments, the subject is a primate. In yet other embodiments, the subject is a human.

As used herein, the term “a therapeutically effective amount” refers to an amount of the antibody with thiol group site-specific modifications, such as the ADC of the present application, that will elicit the biological or medical response of a subject, for example, ameliorate symptoms, alleviate conditions, slow or delay disease progression, or prevent a disease, etc. The therapeutically effective amount will vary with the type and severity of the condition to be alleviated. It is to be further understood that for any particular subject, specific dosage regimens should be adjusted over time according to the individual need and the professional judgment of the person administering or supervising the administration of the compositions. In some embodiments, the therapeutically effective amount is based on a variety of factors, such as the type of disease, the age, weight, sex, medical condition of the patient, the severity, of the condition, the route of administration, and the particular antibody employed. In some embodiments, the therapeutically effective amount can vary widely, but can be determined routinely using standard methods. In some embodiments, the therapeutically effective amount can be adjusted based on the pharmacokinetic or pharmacodynamic parameters, which may include clinical effects such as toxic effects and/or laboratory values.

It will be readily apparent to those skilled in the art that other suitable modifications and adaptations of the present application described herein are obvious and may be made using suitable equivalents without departing from the scope of the disclosure or the embodiments disclosed herein. Having now described the disclosure in detail, the same will be more clearly understood by reference to the following examples, which are included for purposes of illustration only and are not intended to be limiting. Further, unless specifically described otherwise, the reagent and the solvent described in the description can be easily obtained from a commercial supplier.

### EXAMPLES

The invention is further described in the following examples, which do not limit the scope of the invention described in the claims.

### Reagent and Manufacturer

Trastuzumab is commercially available from Roche.

Sacituzumab and Belantamab are commercially available from MedChemExpress.

TCEP is commercially available from Bidepharm.

EDTA is commercially available from Aladdin.

DMA (Dimethylacetamide) is commercially available from Aldrich Sigma.

MC-VC-PAB-MMAE is commercially available from Levena biopharma.

MC-GGFG-DXd is commercially available from Levena.

Dibromomaleimide is commercially available from Aladdin.

Desalting column (type: 40K, 0.5 mL, REF: 87766, Lot SJ251704) is commercially available from Thermo Scientific.

The reagents used in examples, include but not limited to 1-Hydroxybenzotriazole (HOBT), Dimethylacetamide (DMA), 1-(3-Dimethylaminopropyl)-3-ethylcarbodiimide hydrochloride (EDC), N,N-Diisopropylethylamine (DIPEA), ethyl acetate (EtOAc), N, N-Dimethylformamide (DMF) Bicyclic amidine (DBU), 2-(7-Azabenzotriazol-1-yl)-N, N, N′, N′-tetramethyluronium hexafluorophosphate (HATU), N-(3-dimethylaminopropyl)-N′-ethylcarbodiimide hydrochloride (EDCI), trifluoroacetic acid (TFA), dichloromethane (DCM), tert-butylchlorodiphenylsilane (TBDPSCl) are commercially available,

### Synthesis Procedure a of the Compound According to the Present Application

To a solution of TCEP (286.6 mg, 1.0 mmol, 2.0 eq.) in DMF (3 mL) was added HATU (190 mg, 0.5 mmol, 1 eq) followed by DIPEA (2.0 mmol, 4.0 eq) under N₂ atmosphere. The mixture was stirred for 30 min and the amine reagent (0.5 mmol, depending on the structure of the compound) was added. The reaction was stirred at room temperature for 1 h. The reaction mixture was purified by RP-HPLC using a C18 column yielding the desired product.

### Synthesis Procedure A-1 of the Compound According to the Present Application

The product prepared by the synthesis procedure A was dissolved in DCM (3 mL) and TFA (0.3 mL) was added. The mixture was stirred for 1 h, LCMS showed reaction was completed. The mixture was concentrated and the residue was taken up by distilled water, washed with EtOAc twice. The aqueous layer was lyophilized to give corresponding product.

### Synthesis Procedure B of the Compound According to the Present Application

To a solution of TCEP (286.6 mg, 1.0 mmol, 2.0 eq.) in DMF (3 mL) was added HOBt (67.5 mg, 0.5 mmol, 1 eq) and EDCI (95.5 mg, 0.5 mmol, 1.0 eq), followed by DIPEA (2.0 mmol, 4.0 eq) under N₂ atmosphere. The mixture was stirred for 30 min and the amine reagent (0.5 mmo, depending on the structure of the compound) was added. The reaction was stirred at room temperature for 1 h. The reaction mixture was purified by RP-HPLC using a C18 column yielding the corresponding product.

### Synthesis Procedure B-1 of the Compound According to the Present Application

The product prepared by the synthesis procedure B was dissolved in DCM (3 mL) and TFA (0.3 mL) was added. The mixture was stirred for 1 h, LCMS showed reaction was completed. The mixture was concentrated and the residue was taken up by distilled water, washed with EtOAc twice. The aqueous layer was lyophilized to give corresponding product.

### Example 1: Synthesis of TCEP-NO

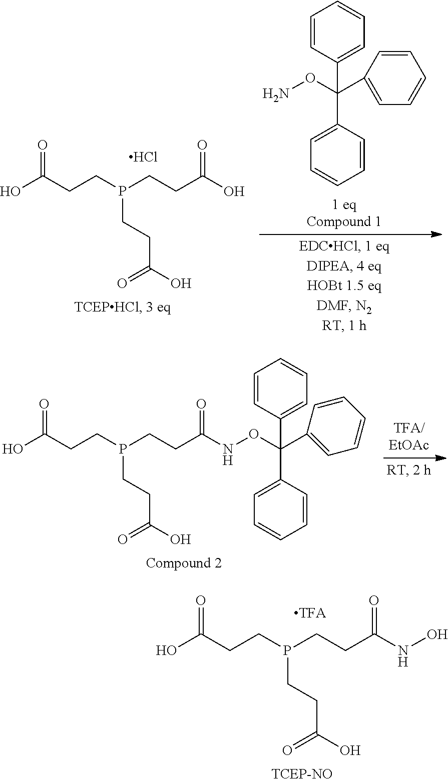 [SMILES: O=C(O)CCP(CCC(=O)O)CCC(=O)O ; Cl ; NOC(c1ccccc1)(c1ccccc1)c1ccccc1 ; O=C(O)CCP(CCC(=O)O)CCC(=O)NOC(c1ccccc1)(c1ccccc1)c1ccccc1 ; O=C(O)CCP(CCC(=O)O)CCC(=O)NO]

Compound 1 O-trithylhydroxylamine (NH₂—O-Trt, 176 μmol, 48.5 mg, 1 eq.), EDC (176 μmol 33.7 mg, 1 eq.) and HOBt (352 μmol, 53.8 mg, 2 eq.) were dissolved in 1.5 mL DMF (degassed) under an inert atmosphere. This solution was added to TCEP (528 μmol, 150 mg, 3 eq.) dissolved in 1.5 mL degassed DMF containing DIPEA (704 μmol, 123 μl, 4 eq.) under an inert atmosphere. The reaction was stirred at room temperature for 60 min. After that, DMF was removed in vacuo and the residue was added CH₃COOH (0.1 N, 5 ml) and EtOAc (2 ml). The resulting mixture was stirred for 5 min and filtered to yield Compound 2 as a white solid (61.2 mg, 12.2%), without further purification which was added EtOAc (2 ml), 5% TFA and 5% TIPS. The reaction was continued for another 2 h. Then H₂O (5 ml) and EtOAc (10 ml) were introduced. The aqueous phase was further washed with EtOAc (10 ml) twice and concentrated to afford TCEP-NO (13 mg). For Compound 2 (also called TCEP-19-int1), MS[M−H]⁻=506.2, Exact mass calc. for C₂₈H₃₀NO₆P is 507.18. ¹H NMR (400 MHz, DMSO-d6): δ 7.40-7.15 (m, 15H), 2.48-2.38 (m, 4H), 2.22 (s, 1H), 2.12-1.52 (m, 7H). For TCEP-NO, MS [M−H]⁻=263.94, exact mass calc. for C9H₁₆NO₆P is 265.07. ¹H-NMR (400 MHz, Deuterium Oxide): δ 2.93-2.85 (m, 4H), 2.73-2.56 (m, 8H).

### Example 2: Synthesis of TCEP-3NO

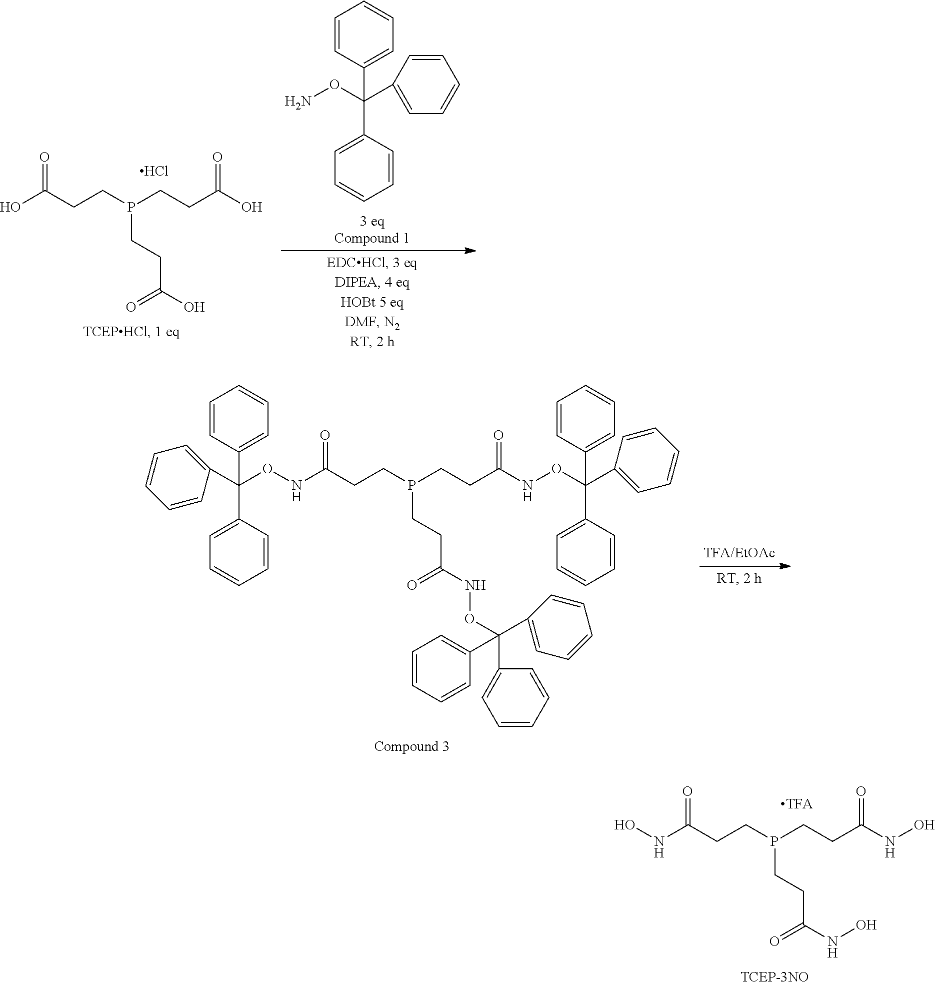 [SMILES: O=C(O)CCP(CCC(=O)O)CCC(=O)O ; Cl ; NOC(c1ccccc1)(c1ccccc1)c1ccccc1 ; O=C(CCP(CCC(=O)NOC(c1ccccc1)(c1ccccc1)c1ccccc1)CCC(=O)NOC(c1ccccc1)(c1ccccc1)c1ccccc1)NOC(c1ccccc1)(c1ccccc1)c1ccccc1 ; O=C(CCP(CCC(=O)NO)CCC(=O)NO)NO]

Compound 1 O-Trithylhydroxylamine (NH₂—O-Trt, 528 μmol, 145.5 mg, 3 eq.), EDC (528 μmol 101 mg, 3 eq.) and HOBt (880 μmol, 134.5 mg, 5 eq.) were dissolved in 4 mL DMF (degassed) under an inert atmosphere. This solution was added to TCEP (176 μmol, 50 mg, 1 eq.) dissolved in 4 mL degassed DMF containing DIPEA (704 μmol, 123 μl, 4 eq.) under an inert atmosphere. The reaction was stirred at room temperature for 60 min. After that, DMF was removed in vacuo and the residue was added CH₃COOH (0.1 N, 5 ml) and EtOAc (5 ml). The resulting mixture was stirred for 5 min and filtered to yield Compound 3 as a white solid, without further purification which was added EtOAc (2 ml), 5% TFA and 5% TIPS. The reaction was continued for another 2 h. Then H₂O (5 ml) and EtOAc (10 ml) were introduced. The aqueous phase was further washed with EtOAc (10 ml) twice and concentrated to afford TCEP-NO (32 mg). For TCEP-3NO, MS [M+H]⁺=295.87, exact mass calc. for C9H₁₈N₃O₆P is 295.09. ¹H NMR (400 MHz, Deuterium Oxide): δ 3.08-2.38 (m, 10H), 2.24-2.17 (m, 5.6 Hz, 2H).

### Example 3: Synthesis of TCEP-CO

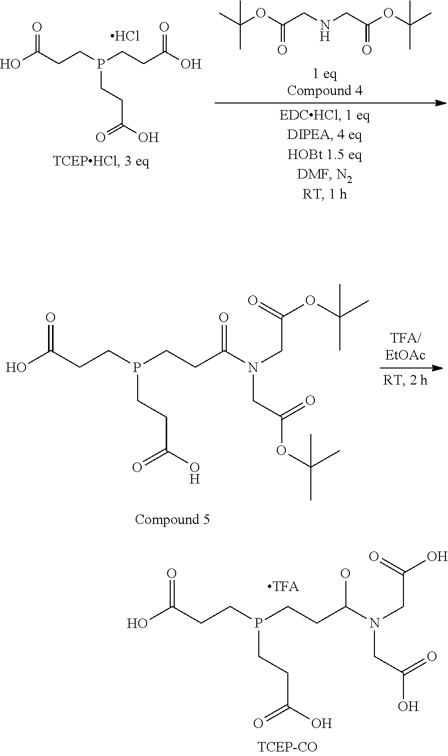 [SMILES: O=C(O)CCP(CCC(=O)O)CCC(=O)O ; Cl ; CC(C)(C)OC(=O)CNCC(=O)OC(C)(C)C ; CC(C)(C)OC(=O)CN(CC(=O)OC(C)(C)C)C(=O)CCP(CCC(=O)O)CCC(=O)O ; O=C(O)CCP(CCC(=O)O)CCC(O)N(CC(=O)O)CC(=O)O]

Compound 4 (176 μmol, 43.2 mg, 1 eq, di-tert-butyl iminodiacetate, Bidepharm), EDC (176 μmol 33.7 mg, 1 eq.) and HOBt (352 μmol, 53.8 mg, 2 eq.) were dissolved in 1.5 mL DMF (degassed) under an inert atmosphere. This solution was added to TCEP (528 μmol, 150 mg, 3 eq.) dissolved in 1.5 mL degassed DMF containing DIPEA (704 μmol, 123 ul, 4 eq.) under an inert atmosphere. The reaction was stirred at room temperature for 60 min. After that, DMF was removed in vacuo and the residue was added CH₃COOH (0.1 N, 5 ml) and EtOAc (2 ml). The resulting mixture was stirred for 5 min and filtered to yield Compound 5 as a white solid, without further purification which was added EtOAc (2 ml), 5% TFA and 5% TIPS. The reaction was continued for another 2 h. Then H₂O (5 ml) and EtOAc (10 ml) were introduced. The aqueous phase was further washed with EtOAc (10 ml) twice and concentrated to afford TCEP-CO (8 mg). For TCEP-CO, MS [M−H]⁻=363.86, exact mass calc. for C₉H₁₆NO₆P is 365.09. ¹H NMR (400 MHz, Deuterium Oxide): δ 4.28 (s, 2H), 4.13 (s, 2H), 2.97 (dt, J=20.0, 6.4 Hz, 2H), 2.83 (dt, J=18.3, 6.9 Hz, 4H), 2.57-2.48 (m, 6H).

### Example 4: Synthesis of TCEP-1

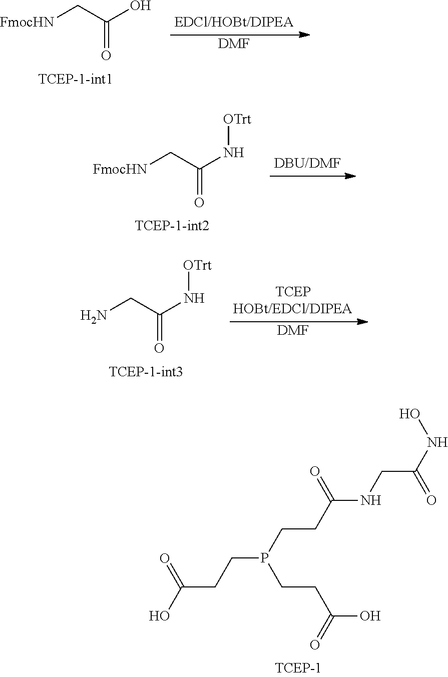

1. TCEP-1-int2

To a solution of TCEP-1-int1 (3.0 g, 10.0 mmol, 1.0 eq, Fmoc-Glycine, Bidepharm) in DMF (30 mL) was added HOBt (1.64 g, 12.0 mmol, 1.2 eq) and EDCI (2.32 g, 12 mmol, 1.2 eq), followed by DIPEA (4.4 mL, 25.2 mmol, 2.5 eq) under N2 atmosphere. The mixture was stirred for 30 min and the Compound 1 O-Trithylhydroxylamine (2.78 g, 10 mmol, 1.0 eq, Bidepharm) was added. The reaction mixture was stirred at room temperature for 1 h and poured into ice-water. The precipitate was collected by filtration and washed with water. The filter cake was dried over vacuum to give the crude product TCEP-1-int2 (4.5 g, 80% yield, white solid), which was used in next step directly without further purification.

2. TCEP-1-int3

To a solution of TCEP-1-int2 (4.5 g, 8.1 mmol, 1.0 eq) in DMF (25 mL) was added DBU (5 mL). The resulting mixture was stirred for 0.5 h at room temperature, LCMS showed reaction was completed. The mixture was poured into ice-water, and extracted with EtOAc, dried over vacuum and purified with flash column (EtOAc/petroleum ether-50%) to give product TCEP-1-int3 (2.0 g, 77% yield, white solid).

### 3. TCEP-1

TCEP-1 was synthesized as the synthesis procedure B-1 wherein TCEP-1-int3 was the amine reagent, yielding TCEP-1 (45.1 mg, 28%) as white solid. MS [M−H]⁻=321.15, exact mass calc. for C₁₁H₁₉N₂O₇P is 322.25. ¹H-NMR (400 MHz, Deuterium Oxide): δ 3.99 (s, 0.64H), 3.87 (s, 1.34 H), 2.96-2.81 (m, 6H), 2.63-2.56 (m, 6H).

### Example 5: Synthesis of TCEP-2

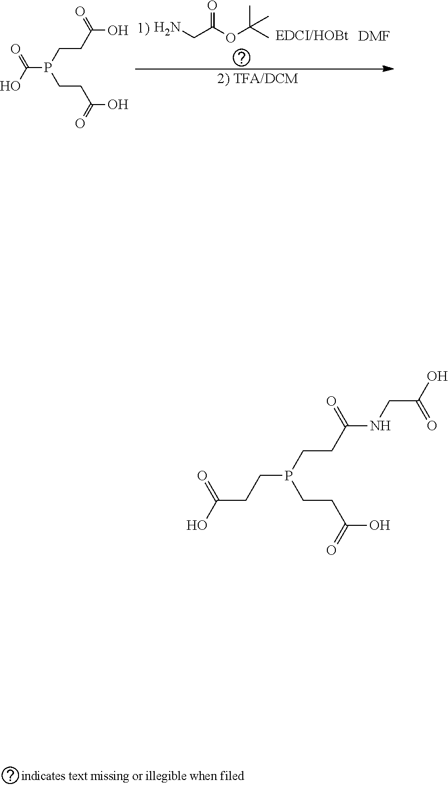

TCEP-2 was synthesized as the synthesis procedure B-1 wherein the Compound 5 (tert-Butyl glycinate, Bidepharm) was the amine reagent, yielding TCEP-2 (52.3 mg, 34%) as white solid. MS [M−H]⁻=306.18, exact mass calc. for C₁₁H₁₈NO₇P is 307.24. ¹H NMR (400 MHz, Deuterium Oxide): 53.99 (s, 2H), 3.00-2.76 (m, 6H), 2.60 (dtd, J=14.0, 7.0, 3.8 Hz, 6H).

### Example 6: Synthesis of TCEP-3

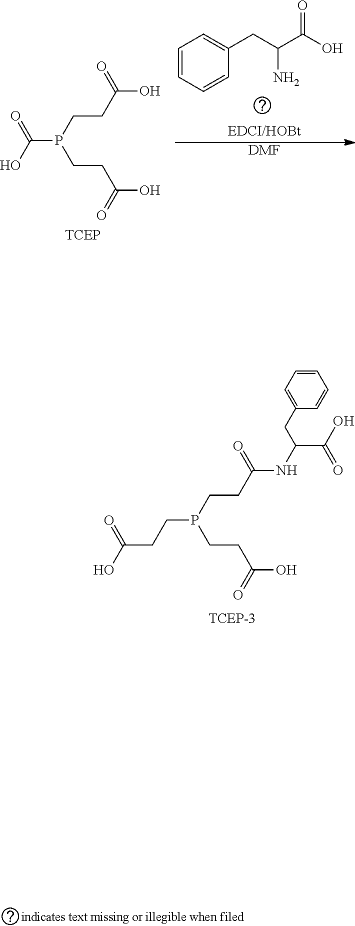

TCEP-3 was synthesized as the procedure B wherein the compound 6 (DL-Phenylalanine, Adamas) is amine reagent, yielding TCEP-3 (65.0 mg, 33%) as white solid. MS [M−H]⁻=396.24, exact mass calc. for C₁₈H₂₄NO₇P is 397.13. ¹H NMR (400 MHz, Deuterium Oxide): δ 7.43-7.25 (m, 5H), 4.71 (dd, J=10.3, 4.8 Hz, 1H), 3.31 (dd, J=13.9, 4.8 Hz, 1H), 2.91 (dd, J=13.9, 10.3 Hz, 1H), 2.86-2.59 (m, 6H), 2.53-2.27 (m, 6H).

### Example 7: Synthesis of TCEP-4

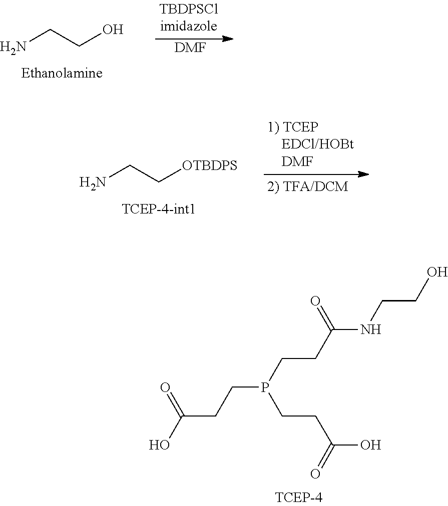

1. TCEP-4-int1

To a solution of Ethanolamine (610 mg, 10 mmol, 1.0 eq, Adamas) in DMC (20 mL) was added imidazole (15 mmol, 1.5 eq) followed by TBDPSCl (10 mmol, 1.0 eq, Adamas) at 0° C. The mixture was stirred for 2 h at room temperature. TLC showed reaction was completed, the reaction mixture was washed with water and brine, organic layer was dried over Na₂SO₄ and filtered. The filtrate was concentrated to give the crude product, which was used in next step directly without further purification.

### 2. TCEP-4

TCEP-4 was synthesized as the synthesis procedure B-1 wherein TCEP-4-int1 was amine reagent, yielding TCEP-4 (60.0 mg, 40.8%) as white solid. MS [M−H]⁻=292.25, exact mass calc. for C₁₁H₂₀NO₆P is 293.10. ¹H NMR (400 MHz, Deuterium Oxide): δ 4.23 (t, J=5.4 Hz, 1H), 3.64 (t, J=5.4 Hz, 1H), 3.48 (t, J=5.3 Hz, 1H), 3.32 (t, J=5.5 Hz, 1H), 2.98-2.78 (m, 6H), 2.60 (dp, J=13.4, 6.8 Hz, 6H).

### Example 8: Synthesis of TCEP-5

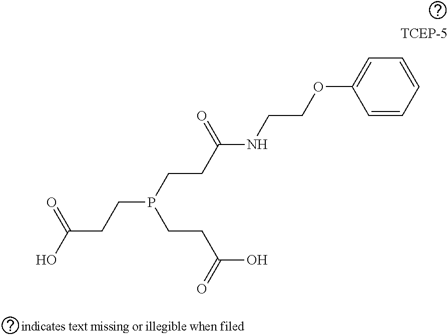 [SMILES: O=C(O)CCP(CCC(=O)O)CCC(=O)NCCOc1ccccc1]

TCEP-5 was synthesized as the procedure B wherein (2-phenoxy-ethylamine, Bidepharm) was amine reagent, yielding TCEP-5 (105.0 mg, 56.8%) as white solid. MS [M−H]⁻=368.24, exact mass calc. for C₁₇H₂₄NO₆P is 369.13. ¹H NMR (400 MHz, Deuterium Oxide): δ 7.34 (dd, J=8.5, 7.2 Hz, 2H), 7.00 (dd, J=19.2, 7.7 Hz, 3H), 4.14 (t, J=5.1 Hz, 2H), 3.56 (t, J=5.1 Hz, 2H), 2.78 (ddt, J=41.6, 20.1, 6.9 Hz, 6H), 2.60-2.43 (m, 6H).

### Example 9: Synthesis of TCEP-6

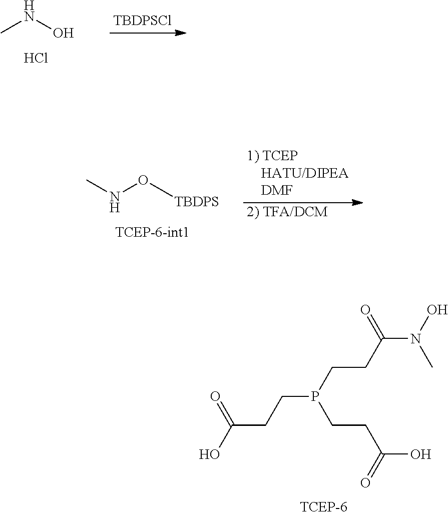

1. TCEP-6-int1

To a solution of N-Methylhydroxylamine hydrochloride (830 mg, 10 mmol, 1.0 eq, Bidepharm) in DMC (20 mL) was added imidazole (15 mmol, 1.5 eq) followed by TBDPSCl (10 mmol, 1.0 eq, Adamas) at 0° C. The mixture was stirred for 2 h at room temperature. TLC showed reaction was completed, the reaction mixture was washed with water and brine, organic layer was dried over Na₂SO₄ and filtered. The filtrate was concentrated to give the crude product, which was used in next step directly without further purification.

### 2. TCEP-6

TCEP-6 was synthesized as the procedure A-1 wherein TCEP-6-int1 was amine reagent, yielding TCEP-6 (13.0 mg, 9.3%) as white solid. MS [M+H]⁺=280.22, exact mass calc. for C₁₀H₁₈NO₆P is 279.09. ¹H NMR (400 MHz, Deuterium Oxide): δ 3.15 (s, 3H), 3.01-2.80 (m, 4H), 2.64-2.45 (m, 6H), 2.17-2.08 (m, 2H).

### Example 10: Synthesis of TCEP-7

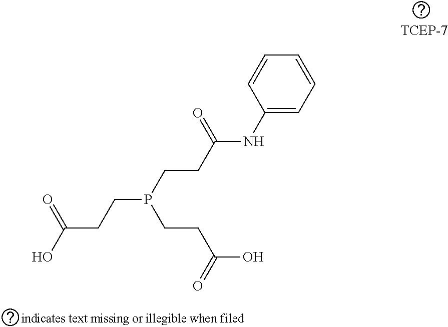 [SMILES: O=C(O)CCP(CCC(=O)O)CCC(=O)Nc1ccccc1]

TCEP-7 was synthesized as the synthesis procedure B wherein (Phenylamine, Adamas) was amine reagent, yielding TCEP-7 (73.0 mg, 45.0% yield) as white solid. MS [M−H]⁻=324.21, exact mass calc. for C₁₅H₂₀NO₅P is 325.11. ¹H NMR (400 MHz, Deuterium Oxide): δ 7.42 (d, J=4.3 Hz, 4H), 7.25 (p, J=4.5 Hz, 1H), 2.96 (ddt, J=32.1, 18.3, 6.9 Hz, 6H), 2.75-2.50 (m, 6H).

### Example 11: Synthesis of TCEP-8

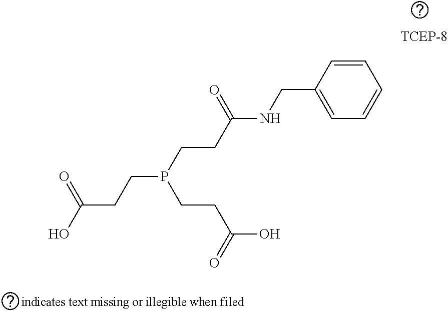 [SMILES: O=C(O)CCP(CCC(=O)O)CCC(=O)NCc1ccccc1]

TCEP-8 was synthesized as the synthesis procedure B wherein (Benzylamine, Adamas) was amine reagent, yielding TCEP-8 (85.6 mg, 50.5% yield) as white solid. MS [M−H]⁻=338.23, exact mass calc. for C₁₆H₂₂NO₅P is 339.12. ¹H NMR (400 MHz, Deuterium Oxide): δ 7.43-7.27 (m, 5H), 4.36 (s, 2H), 2.93-2.77 (m, 6H), 2.63-2.45 (m, 6H).

### Example 12: Synthesis of TCEP-9

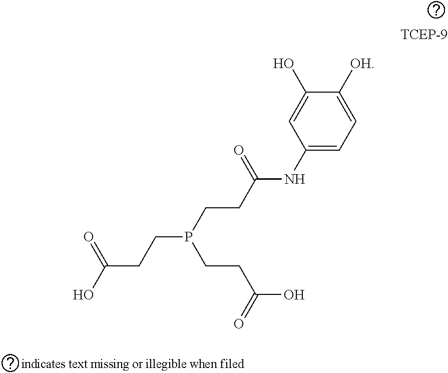 [SMILES: O=C(O)CCP(CCC(=O)O)CCC(=O)Nc1ccc(O)c(O)c1]

TCEP-9 was synthesized as the synthesis procedure A wherein (4-Aminobenzene-1, 2-diol, Bidepharm) was amine reagent, yielding TCEP-9 (63.8 mg, 35.7%) as white solid. MS [M−H]⁻=356.20, exact mass calc. for C₁₅H₂₀NO₇P is 357.10. ¹H NMR (400 MHz, Deuterium Oxide): δ 6.97 (d, J=2.4 Hz, 1H), 6.87 (d, J=8.5 Hz, 1H), 6.78 (dd, J=8.5, 2.5 Hz, 1H), 2.92 (ddt, J=17.8, 10.0, 7.0 Hz, 6H), 2.61 (dq, J=13.9, 6.7 Hz, 6H).

### Example 13: Synthesis of TCEP-10

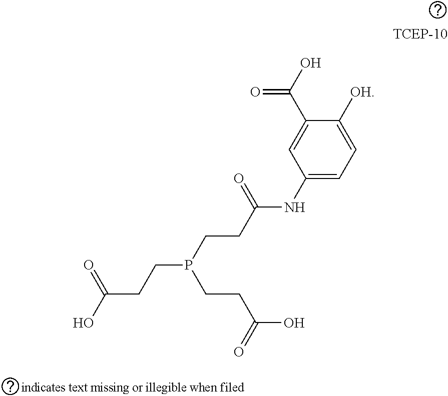 [SMILES: O=C(O)CCP(CCC(=O)O)CCC(=O)Nc1ccc(O)c(C(=O)O)c1]

TCEP-10 was synthesized as the synthesis procedure A wherein (5-Amino-2-hydroxybenzoic acid, Bidepharm) was amine reagent, yielding TCEP-10 (53.7 mg, 27.9%) as white solid. MS [M−H]⁻=384.20, exact mass calc. for C₁₆H₂₀NO₈P is 385.09. ¹H NMR (400 MHz, Deuterium Oxide) S 7.76 (d, J=2.7 Hz, 1H), 7.41 (dd, J=8.9, 2.7 Hz, 1H), 6.89 (d, J=8.9 Hz, 1H), 3.00-2.83 (m, 6H), 2.61 (dq, J=13.9, 6.7 Hz, 6H).

### Example 14: Synthesis of TCEP-11

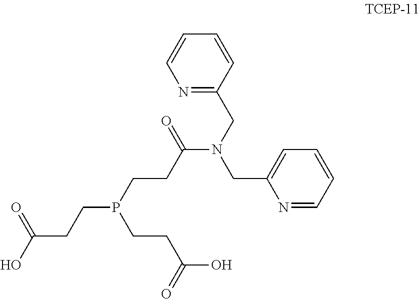 [SMILES: O=C(O)CCP(CCC(=O)O)CCC(=O)N(Cc1ccccn1)Cc1ccccn1]

TCEP-11 was synthesized as the synthesis procedure B wherein (Bis (pyridin-2-ylmethyl) amine, Shanghai Acmec Biochemical Co., Ltd) was amine reagent, yielding TCEP-11 (10.5 mg, 4.9%) as brown solid. MS [M+H]⁺=432.24, exact mass calc. for C₂₁H₂₆N₃O₅P is 431.16. ¹H NMR (400 MHz, Deuterium Oxide) S 8.82-8.68 (m, 2H), 8.58-8.36 (m, 2H), 8.01-7.81 (m, 4H), 5.34 (s, 1H), 5.29 (s, 1H), 5.04 (d, J=2.9 Hz, 2H), 4.48 (s, 1H), 3.16 (dt, J=19.2, 6.5 Hz, 1H), 2.95-2.79 (m, 3H), 2.73-2.48 (m, 5H), 2.20 (ddt, J=36.1, 11.6, 7.5 Hz, 2H).

### Example 15: Synthesis of TCEP-12

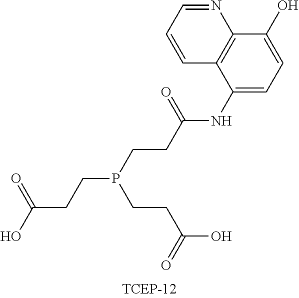 [SMILES: O=C(O)CCP(CCC(=O)O)CCC(=O)Nc1ccc(O)c2ncccc12]

TCEP-12 was synthesized as the synthesis procedure A wherein (5-Amino-8-hydroxyquinoline, Bidepharm) was amine reagent, yielding TCEP-12 (33.2 mg, 16.9%) as white solid. MS [M−H]⁻=391.24, exact mass calc. for C₁₈H₂₁N₂O₆P is 392.11. ¹H NMR (400 MHz, Deuterium Oxide) S 9.02-8.96 (m, 2H), 8.05-7.98 (m, 1H), 7.84 (s, 1H), 7.64 (d, J=8.4 Hz, 1H), 7.44 (d, J=8.4 Hz, 1H), 2.90-2.80 (m, 2H), 2.67-2.56 (m, 6H), 2.26-2.17 (m, 4H).

### Example 16: Synthesis of TCEP-15

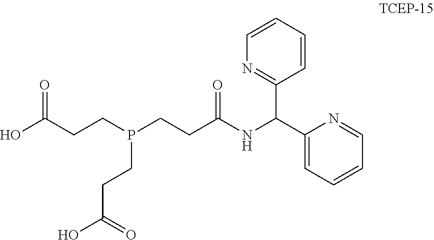 [SMILES: O=C(O)CCP(CCC(=O)O)CCC(=O)NC(c1ccccn1)c1ccccn1]

TCEP-15 was synthesized as the synthesis procedure B wherein (Bis (pyridin-2-yl) methanamine, Bidepharm) was amine reagent, yielding TCEP-15 (21.7 mg, 10.4%) as white solid. MS [M+H]⁺=418.26, exact mass calc. for C₂₀H₂₄N₃O₅P is 417.15. ¹H NMR (400 MHz, Deuterium Oxide) S 8.69 (td, J=6.2, 1.6 Hz, 2H), 8.37 (dtd, J=15.8, 7.9, 1.7 Hz, 2H), 7.93-7.79 (m, 4H), 3.09-2.72 (m, 6H), 2.70-2.52 (m, 6H).

### Example 17: Synthesis of TCEP-18

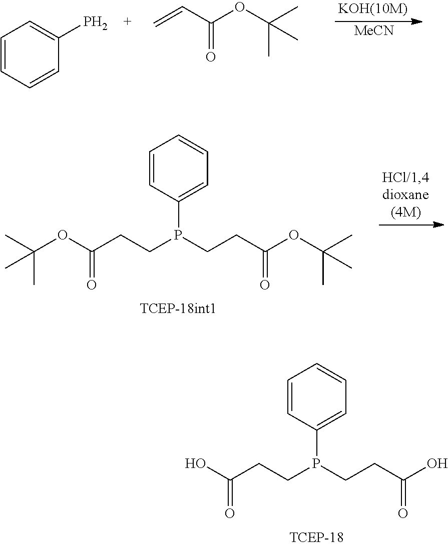

1. TCEP-18-int1

Phenyl phosphine (110 mg, 1.0 mmol, Adamas) was dissolved in acetonitrile (5 ml, degassed) in a flame-dried, round bottom flask under N₂ (g). Potassium hydroxide (ION, 10 ul) was added to this mixture, and the resulting solution was cooled to 0° C. Tert-Butyl acrylate (0.44 ml, 3.0 mmol, Adamas) was added. Upon complete addition of Tert-Butyl acrylate, the reaction was heated at 50° C. and stirred for 8 hours. The reaction mixture was taken up by EtOAc (10 mL), then washed with brine (2×5 ml). The organic layer was dried over Na₂SO₄, filtered, and concentrated in vacuo. The residue was purified by flash column (EtOAc/petroleum ether=0-20% (v/v)) to give product TCEP-18-int1 as a clear liquid (254 mg, 69.4%).

### 2. TCEP-18

The solution of TCEP-18-int1 (254 mg, 0.69 mmol) in HCl/1, 4-dioxane (4M, Adamas) was stirred for 2 h at room temperature under N₂ atmosphere. LCMS showed reaction was completed and the mixture was concentrated to remove 1, 4-dioxane, the resulting residue was taken up by water and lyophilized to give TCEP-18 (152.7 mg, 88.2%) as white solid. MS [M−H]⁻=253.19, exact mass calc. for C₁₂H₁₅O₄P is 254.07. ¹H NMR (400 MHz, DMSO-d₆): δ 7.74 (dd, J=10.9, 7.3 Hz, 2H), 7.62-7.49 (m, 3H), 2.46-2.35 (m, 2H), 2.33-2.00 (m, 6H).

### Example 18: Synthesis of TCEP-19

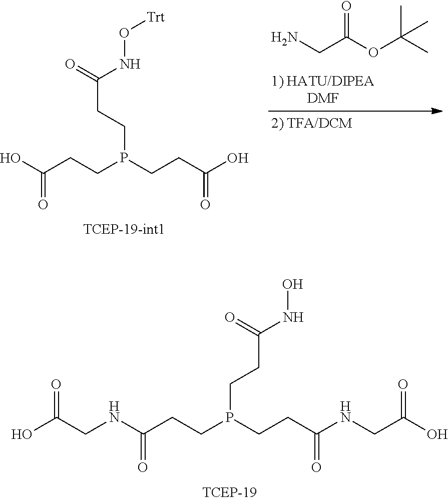

To a solution of TCEP-19-int1 (200 mg, 0.39 mmol, 1.0 eq.) in DMF (3 mL) was added HATU (380 mg, 1.0 mmol, 2.5 eq) followed by DIPEA (174 μL, 1.0 mmol, 2.5 eq) at 0° C. under N₂ atmosphere. The mixture was stirred for 30 min, tert-Butyl glycinate (1 mmol, Adamas) was added. The reaction was stirred at room temperature for 1 h. The reaction mixture was purified by RP-HPLC using a C18 column yielding the protected product. The product was dissolved in DCM (3 mL) and TFA (0.5 mL) was added. The mixture was stirred for 1 h, LCMS showed reaction was completed. The mixture was concentrated and the residue was taken up by distilled water (10 mL), washed with EtOAc (2*5 mL). The aqueous layer was lyophilized to give TCEP-19 (10.2 mg, 6.8%) as brown solid. MS [M+H]⁺=380.24, exact mass calc. for C₁₃H₂₂N₃ O₈P is 379.31. ¹H-NMR (400 MHz, Deuterium Oxide): δ 3.99 (s, 4H), 2.93-2.84 (m, 4H), 2.76-2.53 (m, 8H).

### Example 19: Synthesis of TCEP-20

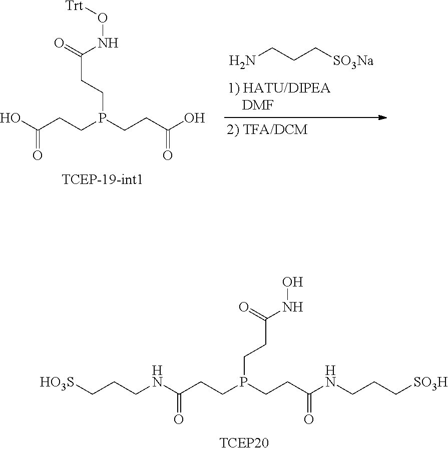

To a solution of TCEP-19-int1 (200 mg, 0.39 mmol, 1.0 eq.) in DMF (3 mL) was added HATU (380 mg, 1.0 mmol, 2.5 eq) followed by DIPEA (174 μL, 1.0 mmol, 2.5 eq) at 0° C. under N₂ atmosphere. The mixture was stirred for 30 min, Sodium 3-Aminopropane-1-Sulfonate (1 mmol, Adamas) was added. The reaction was stirred at room temperature for 1 h. The reaction mixture was purified by RP-HPLC using a C18 column yielding the protected product. The product was dissolved in DCM (3 mL) and TFA (0.5 mL) was added. The mixture was stirred for 1 h, LCMS showed reaction was completed. The mixture was concentrated and the residue was taken up by distilled water (10 mL), washed with EtOAc (2*5 mL). The aqueous layer was lyophilized to give TCEP-20 (23.7 mg, 11.7%) as brown solid. MS[M−H]⁻=506.22, exact mass calc. for C₁₅H₃₀N₃O₁₀PS₂ is 507.51. ¹H NMR (400 MHz, Deuterium Oxide): δ 3.27 (t, J=6.8 Hz, 4H), 2.91-2.85 (m, 4H), 2.80-2.71 (m, 4H), 2.69-2.61 (m, 2H), 2.58-2.49 (m, 6H), 1.94-1.85 (m, 4H).

### Example 20: Synthesis of TCEP-21

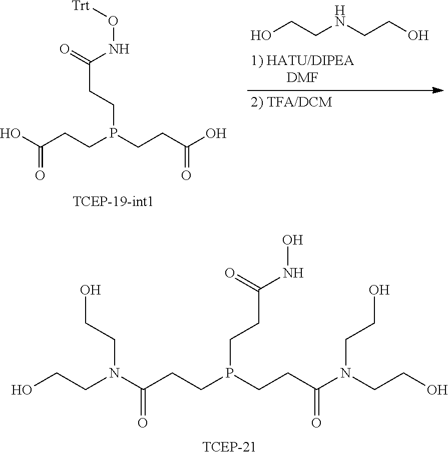

To a solution of TCEP-19-int1 (200 mg, 0.39 mmol, 1.0 eq.) in DMF (3 mL) was added HATU (380 mg, 1.0 mmol, 2.5 eq) followed by DIPEA (174 μL, 1.0 mmol, 2.5 eq) at 0° C. under N₂ atmosphere. The mixture was stirred for 30 min, Diethanolamine (1 mmol, Bidepharm) was added. The reaction was stirred at room temperature for 1 h. The reaction mixture was purified by RP-HPLC using a C18 column yielding the protected product. The product was dissolved in DCM (3 mL) and TFA (0.5 mL) was added. The mixture was stirred for 1 h, LCMS showed reaction was completed. The mixture was concentrated and the residue was taken up by distilled water (10 mL), washed with EtOAc (2*5 mL). The aqueous layer was lyophilized to give TCEP-20 (13.8 mg, 7.8 yield) as white solid. MS [M+H]⁺=440.27, exact mass calc. for C₁₇H₃₄N₃O₈P is 439.45. ¹H NMR (400 MHz, Deuterium Oxide): δ 4.50-4.28 (m, 4H), 3.78 (t, J=5.2 Hz, 4H), 3.38 (t, J=5.1 Hz, 4H), 3.17 (q, J=5.2 Hz, 4H), 2.96-2.80 (m, 4H), 2.68-2.51 (m, 8H).

### Example 21: Synthesis of TCEP-23

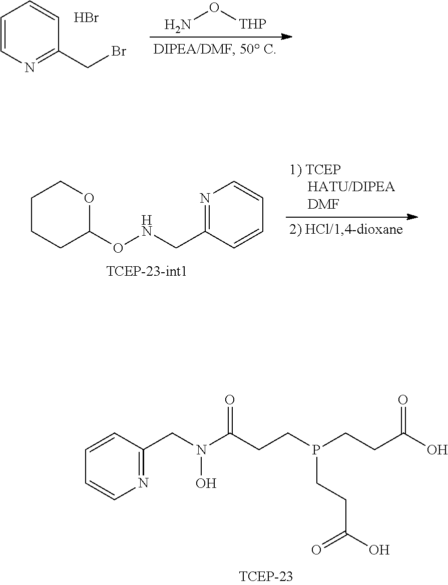

1. TCEP-23-int1

To a solution of 2-(Aminooxy) tetrahydro-2H-pyran (1.17 g, 10 mmol, 2.0 eq, Bidepharm) in DMF (15 mL) was added DIPEA (3.5 mL, 20 mmol, 4. eq) followed by 2-(Bromomethyl) pyridine hydrobromide (1.3 g, 5.0 mmol, 1.0 eq, Adamas). The mixture was stirred for 16 h at 50° C. The reaction mixture was poured into water (100 mL), extracted with EtOAc (30Ml*3). The organic layer was washed with brine (30 mL), dried over Na₂SO₄ and filtered. The filtrate was concentrated and purified by flash column, to give TCEP-23-int1 (800 mg, 80%), as colorless oil.

### 2. TCEP-23

To a solution of TCEP (286.6 mg, 1.0 mmol, 2.0 eq.) in DMF (3 mL) was added HATU (190 mg, 0.5 mmol, 1 eq) followed by DIPEA (2.0 mmol, 4.0 eq) under N₂ atmosphere. The mixture was stirred for 30 min and TCEP-23-int1 (100 mg, 0.5 mmol, 1.0 eq) was added. The reaction was stirred at room temperature for 4 h. The reaction mixture was purified by RP-HPLC using a C18 column yielding the desired product. The product was dissolved in HCl/1, 4-dioxane (3 mL). The mixture was stirred for 1 h, LCMS showed reaction was completed. The mixture was concentrated and the residue was taken up by distilled water, lyophilized to give TCEP-23 (17.9 mg, 10.0%) as white solid. MS [M+H]⁺=357.19, exact mass calc. for C₁₅H₂₁N₂O₆P is 356.31. ¹H-NMR (400 MHz, Deuterium Oxide): δ 8.73 (dd, J=6.3, 1.7 Hz, 1H), 8.59 (td, J=7.9, 1.6 Hz, 1H), 8.02 (dd, J=6.4, 3.4 Hz, 2H), 5.21 (s, 2H), 3.21-2.97 (m, 2H), 2.95-2.80 (m, 4H), 2.69-2.56 (m, 6H).

### Example 22: Synthesis of TCEP-24

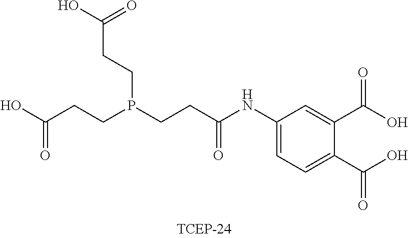 [SMILES: O=C(O)CCP(CCC(=O)O)CCC(=O)Nc1ccc(C(=O)O)c(C(=O)O)c1]

TCEP-24 was synthesized as the synthesis procedure A wherein (4-Aminophthalic acid, Bidepharm) was amine reagent, yielding TCEP-24 (21.5 mg, 10.4% yield) as white solid. MS [M−H]⁻=412.22, exact mass calc. for C₁₇H₂₀NO₉P is 413.09. ¹H NMR (400 MHz, Deuterium Oxide): δ 7.87 (d, J=8.5 Hz, 1H), 7.77 (d, J=2.1 Hz, 1H), 7.71-7.64 (m, 1H), 3.08-3.00 (m, 2H), 2.96-2.86 (m, 4H), 2.70-2.59 (m, 6H).

### Example 23: Synthesis of TCEP-25

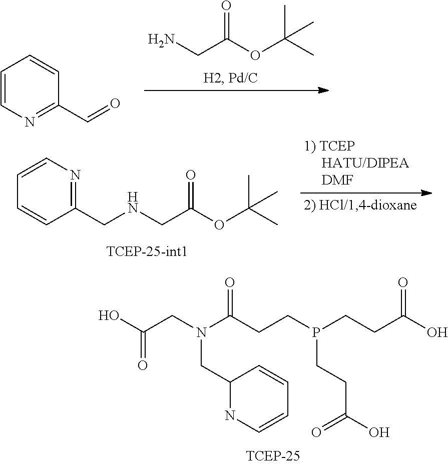

1. TCEP-25-int1

To a solution of 2-Pyridinecarboxaldehyde (1.0 g, 10 mmol, 1.0 eq. Adamas) and tert-Butyl glycinate (1.3 g, 10.0 mmol, 1.0 eq) in MeOH (25 mL) was added Pd/C (150 mg) and two drops of AcOH. The mixture was degassed 3 times and purged with H₂, then stirred for 16 h at room temperature under H₂ atmosphere. The reaction mixture was filtered through a Celite pad and the filtrate was concentrate then purified by flash column, to give TCEP-25-int1 (1.6 g, 72.0%) as yellow oil.

### 2. TCEP-25

To a solution of TCEP (286.6 mg, 1.0 mmol, 2.0 eq.) in DMF (3 mL) was added HATU (190 mg, 0.5 mmol, 1 eq) followed by DIPEA (2.0 mmol, 4.0 eq) under N₂ atmosphere. The mixture was stirred for 30 min and TCEP-25-int1 (111 mg, 0.5 mmol, 1.0 eq) was added. The reaction was stirred at room temperature for 4 h. The reaction mixture was purified by RP-HPLC using a C18 column yielding the desired product. The product was dissolved in HCl/1, 4-dioxane (3 mL). The mixture was stirred for 1 h, LCMS showed reaction was completed. The mixture was concentrated and the residue was taken up by distilled water, lyophilized to give TCEP-25 (51.3 mg, 17.2% yield) as white solid. MS [M+H]⁺=399.25, exact mass calc. for C₁₇H₂₃N₂O₇P is 398.12. ¹H NMR (400 MHz, Deuterium Oxide): δ 8.60 (dd, J=5.9, 1.6 Hz, 1H), 8.45 (td, J=8.0, 1.6 Hz, 1H), 7.92 (d, J=8.3 Hz, 1H), 7.87 (ddd, J=7.5, 5.9, 1.3 Hz, 1H), 4.88 (s, 2H), 4.39 (s, 2H), 2.84-2.68 (m, 6H), 2.51-2.41 (m, 6H).

### Example 24: Synthesis of TCEP-26

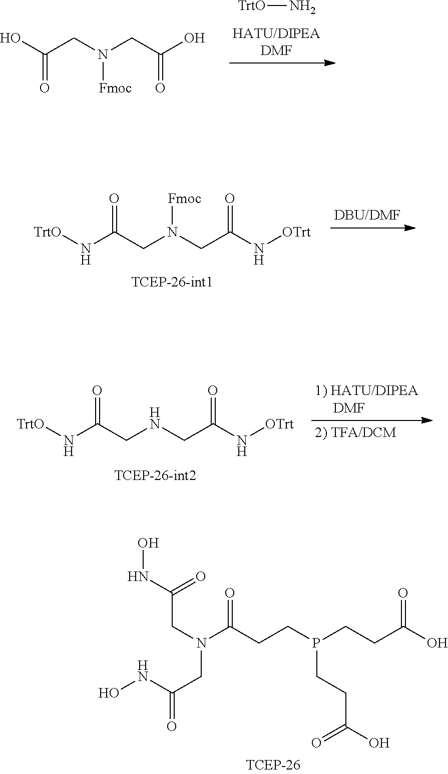

1. TCEP-26-int1

To a solution of Fmoc-iminodiacetic acid (1.8 g, 5.0 mmol, 1.0 eq, Bidepharm) in DMF (30 mL) was added HATU (4.3 g, 11.0 mmol, 2.2 eq) followed by DIPEA (2.0 mmol, 4.0 eq) under N₂ atmosphere. The mixture was stirred for 30 min and 0-Tritylhydroxylamine (3.0 g, 11.0 mmol, 2.2 eq) was added. The reaction was stirred at room temperature for 4 h. The reaction mixture was poured into water (200 mL). The precipitate was collected by filtration and the filter cake was dried over vacuum to give TCEP-23-int1 (4.0 g, 92.0%), as white solid.

2. TCEP-26-int2

To a solution of TCEP-26-int1 (2.0 g, 2.3 mmol, 1.0 eq) in DMF (10 mL) was added DBU (2 mL). The mixture was stirred for 1 h at room temperature, then poured into ice-water (100 mL), extracted with EtOAc (50 mL*3). The combined organic layer was washed with brine, dried over Na₂SO₄ and filtered. The filtrate was concentrated and purified by flash column (EtOAc/petroleum ether=0-50%, v/v) to give TCEP-26-int2 (1.2 g, 80%).

### 3. TCEP-26

The compound was synthesized as the synthesis procedure A-1 wherein TCEP-26-int2 was amine reagent, yielding TCEP-26 (31.5 mg, 21.3% yield) as white solid. MS [M+H]⁺=396.17, exact mass calc. for C₁₃H₂₂N30₉P is 395.11. ¹H NMR (400 MHz, Deuterium Oxide) S 4.13 (s, 2H), 3.97 (s, 2H), 2.85-2.77 (m, 4H), 2.54-2.47 (m, 6H), 2.18-2.08 (m, 2H).

### Example 25: Synthesis of TCEP-28

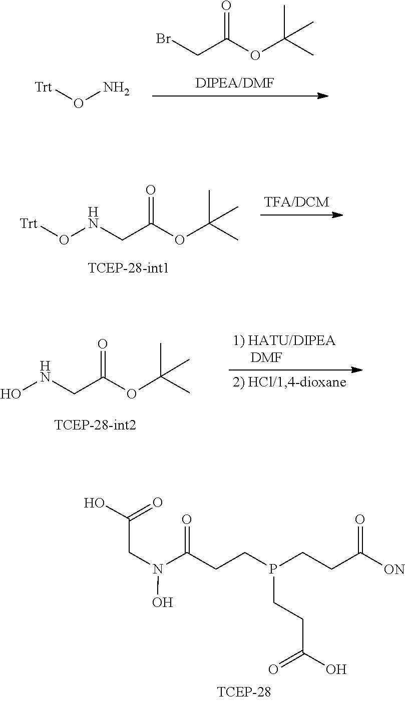

1. TCEP-28-int1

To a solution of O-Tritylhydroxylamine (1.4 g, 5.0 mmol, 1.0 eq) in DMF (15 mL) was added DIPEA (1.7 mL, 10 mmol, 2. eq) followed by tert-Butyl bromoacetate (1.0 g, 5.0 mmol, 1.0 eq, Adamas). The mixture was stirred for 16 h at 50° C. The reaction mixture was poured into water (100 mL), extracted with EtOAc (30 mL*3). The organic layer was washed with brine (30 mL), dried over Na₂SO₄ and filtered. The filtrate was concentrated and purified by flash column, to give TCEP-28-int1 (1.4 g, 70%), as white solid.

2. TCEP-28-int2

To a solution of TCEP-28-int1 (1.4 g, 3.6 mmol, 1.0 eq) in DCM (15 mL) was added TFA (1.5 mL). The mixture was stirred for 2 h at room temperature. The reaction mixture was concentrated and purified by flash column, to give TCEP-28-int2 (380 mg, 71.8%), as colorless oil.

### 3. TCEP-28

To a solution of TCEP (286.6 mg, 1.0 mmol, 2.0 eq.) in DMF (3 mL) was added HATU (190 mg, 0.5 mmol, 1 eq) followed by DIPEA (2.0 mmol, 4.0 eq) under N₂ atmosphere. The mixture was stirred for 30 min and TCEP-28-int2 (73.5 mg, 0.5 mmol, 1.0 eq) was added. The reaction was stirred at room temperature for 2 h. The reaction mixture was purified by RP-HPLC using a C18 column yielding the desired product. The product was dissolved in HCl/1, 4-dioxane (3 mL, Adamas). The mixture was stirred for 1 h, LCMS showed reaction was completed. The mixture was concentrated and the residue was taken up by distilled water, lyophilized to give TCEP-28 (43.6 mg, 27.1% yield) as white solid. MS [M−H]=322.16, exact mass calc. for C₁₁H₁₈NO₈P is 323.08. ¹H NMR (400 MHz, Deuterium Oxide): δ 4.36 (s, 2H), 2.86-2.77 (m, 6H), 2.56-2.48 (m, 6H).

### Example 26: Synthesis of TCEPA

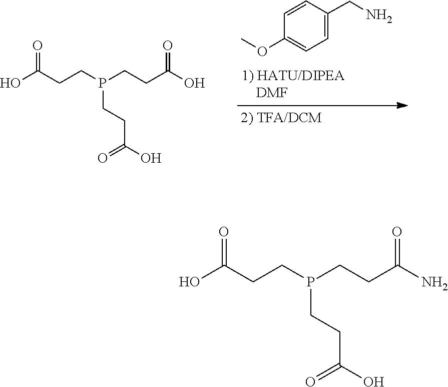

TCEPA was synthesized as the synthesis procedure A-1 wherein 4-methoxybenzylamine was amine reagent, yielding TCEPA (13.5 mg, 11%). MS [M+H]⁺=250.18, exact mass calc. for C₉H₁₆NO₅P is 249.08. ¹H NMR (400 MHz, Deuterium Oxide) S 2.85-2.70 (m, 4H), 2.61-2.43 (m, 6H), 2.15-2.06 (m, 2H).

### Example 27: Synthesis of TCEP-30

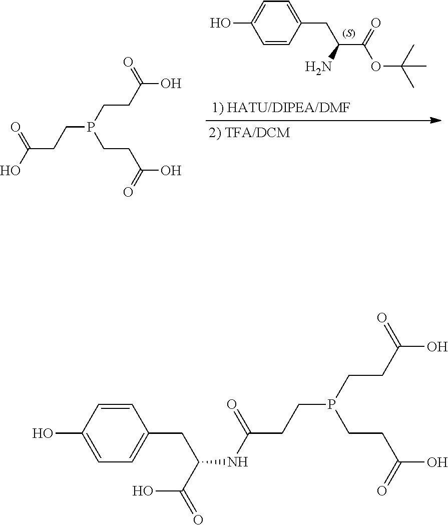

TCEP-30 was synthesized as the synthesis procedure A-1 wherein [tert-Butyl L-tyrosinate, Adamas) was amine reagent, yielding TCEP-30 (25.3 mg, 12.25%) as white solid. MS [M+H]⁺=414.23, exact mass calc. for C₁₈H₂₄NO₈P is 413.12. ¹H NMR (400 MHz, Deuterium Oxide): δ 7.24-7.14 (m, 2H), 6.91-6.82 (m, 2H), 4.67 (dd, J=10.3, 4.7 Hz, 1H), 3.26 (dd, J=14.0, 4.7 Hz, 1H), 2.92-2.64 (m, 7H), 2.57-2.30 (m, 6H).

### Example 28: Synthesis of TCEP-31

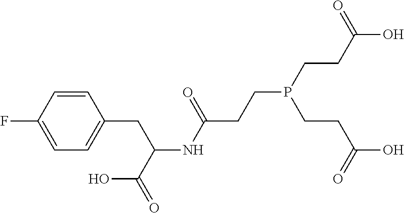 [SMILES: O=C(O)CCP(CCC(=O)O)CCC(=O)NC(Cc1ccc(F)cc1)C(=O)O]

TCEP-31 was synthesized as the synthesis procedure A wherein (DL-3-(4-Fluorophenyl) alanine, Bidepharm) was amine reagent, yielding TCEP-31 (27.1 mg, 13.10%) as white solid. MS [M+H]⁺=416.01, exact mass calc. for C18H₂₃NO₇P is 415.12. ¹H NMR (400 MHz, Deuterium Oxide): δ 7.36-7.26 (m, 2H), 7.12 (t, J=8.8 Hz, 2H), 4.70 (dd, J=10.1, 4.8 Hz, 1H), 3.31 (dd, J=14.0, 4.9 Hz, 1H), 2.95 (dd, J=14.0, 10.1 Hz, 1H), 2.90-2.65 (m, 6H), 2.56-2.40 (m, 6H).

### Example 29: Synthesis of TCEP-32

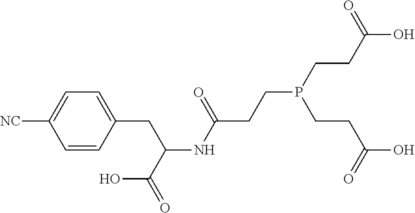 [SMILES: N#Cc1ccc(CC(NC(=O)CCP(CCC(=O)O)CCC(=O)O)C(=O)O)cc1]

TCEP-32 was synthesized as the synthesis procedure A wherein (DL-4-Cyanophenylalanine, Bidepharm) was amine reagent, yielding TCEP-32 (18.5 mg, 8.76%) as white solid. MS [M+H]⁺=423.24, exact mass calc. for C₁₉H₂₃N₂O₇P is 422.12. ¹H NMR (400 MHz, Deuterium Oxide): δ 7.77 (d, J=8.1 Hz, 2H), 7.49 (d, J=8.1 Hz, 2H), 4.77-4.71 (m, 1H), 3.42 (dd, J=14.0, 5.0 Hz, 1H), 3.05 (dd, J=14.0, 10.0 Hz, 1H), 2.91-2.70 (m, 6H), 2.68-2.39 (m, 6H).

### Example 30: Synthesis of TCEP-33

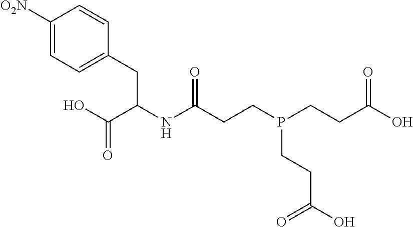 [SMILES: O=C(O)CCP(CCC(=O)O)CCC(=O)NC(Cc1ccc([N+](=O)[O-])cc1)C(=O)O]

TCEP-33 was synthesized as the synthesis procedure A wherein (DL-4-nitro-phenylalanine, Bidepharm) was amine reagent, yielding TCEP-33 (20.7 mg, 9.37%) as white solid. MS [M+H]⁺=443.24, exact mass calc. for C₁₉H₂₃N₂O₉P is 442.11. ¹H NMR (400 MHz, Deuterium Oxide): δ 8.22 (d, J=8.2 Hz, 2H), 7.54 (d, J=8.2 Hz, 2H), 4.84-4.80 (m, 1H), 3.47 (dd, J=14.0, 4.8 Hz, 1H), 3.11 (dd, J=13.9, 10.3 Hz, 1H), 2.91-2.73 (m, 6H), 2.58-2.38 (m, 6H).

### Example 31: Synthesis of TCEP-34

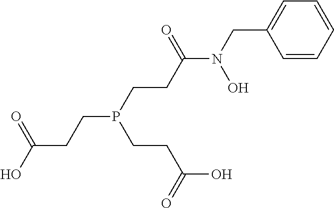 [SMILES: O=C(O)CCP(CCC(=O)O)CCC(=O)N(O)Cc1ccccc1]

TCEP-34 was synthesized as the synthesis procedure A wherein (N-Benzylhydroxylamine hydrochloride, Bidepharm) was amine reagent, yielding TCEP-34 (15.7 mg, 8.85%) as white solid. MS [M+H]⁺=356.05, exact mass calc. for C₁₆H₂₂NO₆P is 355.12. ¹H NMR (400 MHz, Deuterium Oxide): δ 7.55-7.34 (m, 5H), 4.84 (s, 2H), 3.14 (dt, J=19.8, 6.9 Hz, 2H), 2.92 (dt, J=18.2, 7.0 Hz, 4H), 2.62 (dq, J=14.5, 7.3 Hz, 6H).

### Example 32: Synthesis of TCEP-35

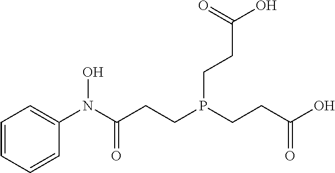 [SMILES: O=C(O)CCP(CCC(=O)O)CCC(=O)N(O)c1ccccc1]

TCEP-35 was synthesized as the synthesis procedure A wherein (N-Phenylhydroxylamine, Bidepharm) was amine reagent, yielding TCEP-35 (17.1 mg, 10.0%) as white solid. MS [M+H]⁺=341.97, exact mass calc. for C₁₅H₂NO₆P is 341.10. ¹H NMR (400 MHz, Deuterium Oxide): δ 7.57-6.88 (m, 5H), 3.27-3.22 (m, 1H), 2.92-2.78 (m, 3H), 2.65-2.53 (m, 6H), 2.29-1.98 (m, 2H).

### Example 33: Synthesis of TCEP-37

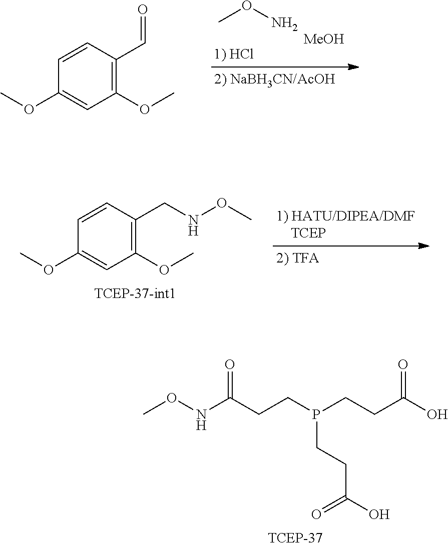

1. TCEP-37-int1

To a solution of 2, 4-Dimethoxybenzaldehyde (1.66 g, 10.0 mmol, 1.0 eq, Adamas) in MeOH (25 mL) was added O-methylhydroxylamine hydrochloride (1.66 g, 20.0 mmol, 2.0 eq, Bidepharm). The resulting mixture was stirred for 16 h at room temperature, LCMS showed reaction was completed. The mixture was concentrated and the residue was taken up by AcOH (20 mL), NaBH₃CN was added and stirred for 5 h at room temperature. LCMS showed reaction was completed. The reaction mixture was concentrated and residue was poured into ice-water (200 mL) extracted with EtOAc (50 mL*3). The combined organic layer was dried over Na₂SO₄ and filtered. The filtrate was concentrated over vacuum and purified with flash column (EtOAc/petroleum ether=0-50%) to give product TCEP-37-int1 (N-(2, 4-dimethoxybenzyl)-O-methyl hydroxylamine, 1.5 g, 76.1%, colorless oil).

### 2. TCEP-37

TCEP-37 was synthesized as the synthesis procedure A-1 wherein TCEP-73-int1 was amine reagent, yielding TCEP-37 (12.8 mg, 9.14%) as white solid. MS [M+H]⁺=280.18, exact mass calc. for C₁₀H₁₈NO₆P is 279.09. ¹H NMR (400 MHz, Deuterium Oxide): δ (s, 3H), 2.89 (dt, J=18.4, 7.1 Hz, 4H), 2.72 (dd, J=18.1, 6.6 Hz, 2H), 2.61 (dq, J=13.9, 6.7 Hz, 6H).

### Examples 34-66: Preparation of ADCs with D2

The ADC is prepared in a one-pot reaction:
(1) ZnCl₂ (0.24 mM) and reductant (0.02 mMv) were added to a solution of a monoclonal antibody (0.012 mM, in MES buffer, pH6.7, 20 mM) and the reaction mixture was allowed to stay at 4° C. for 4 h, 8 h, and 12 h, respectively;
(2) EDTA (0.6 mMv) was added to trap Zn²⁺;
(3) MC-VC-PAB-MMAE (0.06 mM) in DMA was introduced and the reaction was continued at 24° C. for 1 h;
(4) Cysteine (0.08 mM) was added to deplete excessive MC-VC-PAB-MMAE;
(5) The reaction mixture was subjected to purification using a desalting column.

The monoclonal antibody and reductant used, the molar ratio of the antibody and reductant, and the incubation time in step (1) are as follows. Meanwhile, the buffer system is MOPS buffer and the pH value is 7.4 in example 40.

| | | | | | The |
|---|---|---|---|---|---|
| | | | | Antibody/the | incubation |
| | | | | reductant | time in |
| | Serial | | | (molar | step |
| Example | NO. | Antibody | Reductant | ratio) | (1) (h) |
| 34 | 537 | Trastuzumab | TCEP-NO | 1:1.67 | 4 |
| 35 | 542 | Sacituzumab | TCEP-NO | 1:1.67 | 4 |
| 36 | 511 | Belantamab | TCEP-NO | 1:1.67 | 4 |
| 37 | 616 | Trastuzumab | TCEP-3NO | 1:1.67 | 8 |
| 38 | 678 | Sacituzumab | TCEP-3NO | 1:1.67 | 8 |
| 39 | 680 | Belantamab | TCEP-3NO | 1:1.67 | 8 |
| 40 | 660 | Trastuzumab | TCEP-CO | 1:1.67 | 12 |
| 41 | 681 | Sacituzumab | TCEP-CO | 1:1.67 | 12 |
| 42 | 682 | Belantamab | TCEP-CO | 1:1.67 | 12 |
| 43 | / | Trastuzumab | TCEP-1 | 1:1.3 | 4 |
| 44 | / | Trastuzumab | TCEP-2 | 1:1.2 | 4 |
| 45 | / | Trastuzumab | TCEP-3 | 1:1.2 | 4 |
| 46 | / | Trastuzumab | TCEP-4 | 1:0.9 | 4 |
| 47 | / | Trastuzumab | TCEP-5 | 1:1.6 | 4 |
| 48 | / | Trastuzumab | TCEP-6 | 1:1.6 | 4 |
| 49 | / | Trastuzumab | TCEP-7 | 1:0.9 | 4 |
| 50 | / | Trastuzumab | TCEP-8 | 1:2.2 | 4 |
| 51 | / | Trastuzumab | TCEP-9 | 1:1.4 | 4 |
| 52 | / | Trastuzumab | TCEP-10 | 1:1.8 | 4 |
| 53 | / | Trastuzumab | TCEP-15 | 1:1 | 4 |
| 54 | / | Trastuzumab | TCEP-18 | 1:1 | 4 |
| 55 | / | Trastuzumab | TCEP-19 | 1:1.4 | 4 |
| 56 | / | Trastuzumab | TCEP-20 | 1:1.6 | 4 |
| 57 | / | Trastuzumab | TCEP-21 | 1:1.4 | 4 |
| 58 | / | Trastuzumab | TCEP-23 | 1:1.2 | 4 |
| 59 | / | Trastuzumab | TCEP-24 | 1:1.2 | 4 |
| 60 | / | Trastuzumab | TCEP-25 | 1:1.7 | 4 |
| 61 | / | Trastuzumab | TCEP-26 | 1:1.1 | 4 |
| 62 | / | Trastuzumab | TCEP-28 | 1:1.7 | 4 |
| 63 | / | Trastuzumab | TCEPA | 1:2.3 | 4 |
| 64 | / | Trastuzumab | TCEP-34 | 1:1.3 | 4 |
| 65 | / | Trastuzumab | TCEP-35 | 1:1.6 | 4 |
| 66 | / | Trastuzumab | TCEP-37 | 1:1.2 | 4 |

### Examples 67-81 and Comparative Example 10: Preparation of Trastuzumab-[MC-VC-PAB-MMAE]₂ Conjugate with Different Molar Ratio of the ZnCl₂ and the Reductant

The preparation of Trastuzumab-[MC-VC-PAB-MMAE]₂ conjugate is similar to the preparation of the ADC with D2 of example 34, but it adjusts the dosage of ZnCh₂ in step (1). The dosage of ZnCh₂ and the molar ratio of the ZnCh₂ and the reductant are as follows:

| No. | Serial NO. | ZnCl₂ (mM) | ZnCl₂/TCEP-NO (Molar Ratio) |
|---|---|---|---|
| E67 | 632 | 0.008 | 0.4 |
| E68 | 633 | 0.02 | 1 |
| E69 | 599 | 0.04 | 2 |
| E70 | 600 | 0.08 | 4 |
| E71 | 601 | 0.12 | 6 |
| E72 | 602 | 0.16 | 8 |
| E73 | 603 | 0.2 | 10 |
| E74 | 605 | 0.28 | 14 |
| E75 | 606 | 0.32 | 16 |
| E76 | 729 | 0.48 | 24 |
| E77 | / | 0.6 | 30 |
| E78 | 733 | 1.2 | 60 |
| E79 | / | 1.4 | 70 |
| E80 | / | 2.5 | 125 |
| E81 | / | 4 | 200 |
| C10 | / | 5 | 250 |

“E” was short for Example, and “C” was short for comparative example in the application.

### Examples 82-85: Preparation of Trastuzumab-[MC-VC-PAB-MMAE]₂ Conjugate with Different Molar Ratio of the Antibody and the Reductant

The preparation of Trastuzumab-[MC-VC-PAB-MMAE]₂ conjugate is similar to the preparation of ADC with D2 of example 34, but it adjusts the dosage of the antibody in step (1) or the incubation time in step (1). The dosage of antibody and the molar ratio of the antibody and the reductant are as follows:

| | | | Intubation time |
|---|---|---|---|
| | The antibody | The antibody/TCEP-NO | in step |
| Example | (mM) | (Molar Ratio) | (1) (h) |
| 82 | 0.011 | 1:1.8 | 4 |
| 83 | 0.01 | 1:2.0 | 1 |
| 84 | 0.008 | 1:2.5 | 1 |
| 85 | 0.0067 | 1:3.0 | 1 |

### Examples 86-101: Preparation of Trastuzumab-[MC-VC-PAB-MMAE]₂ Conjugate in Different Buffers

The preparation of Trastuzumab-[MC-VC-PAB-MMAE]₂ conjugate is similar to the preparation of ADC of example 34, but it adjusts the buffer as follows:

| Example | Serial NO. | The buffer system | pH |
|---|---|---|---|
| 86 | 561 | Bis-Tris buffer | 6.7 |
| 87 | 562 | PIPES buffer | 6.7 |
| 88 | 563 | MOPS buffer | 6.7 |
| 89 | 564 | BES buffer | 6.7 |
| 90 | 566 | HEPES buffer | 6.7 |
| 91 | 567 | ADA buffer | 6.7 |
| 92 | 568 | PB | 6.7 |
| 93 | 626 | DIPSO buffer | 7.4 |
| 94 | 627 | MOBS buffer | 7.4 |
| 95 | 628 | MOPSO buffer | 7.4 |
| 96 | 629 | TES buffer | 7.4 |
| 97 | 630 | ACES buffer | 7.4 |
| 98 | 631 | TAPSO buffer | 7.4 |
| 99 | / | MES buffer | 5.8 |
| 100 | / | MES buffer | 6.4 |
| 101 | / | MES buffer | 6.7 |

All the buffers are commercially available from Macklin.

### Examples 102-113: Preparation of Trastuzumab-[MC-VC-PAB-MMAE]₂ Conjugate in Different Incubation Temperature or Time in Step (1)

The preparation of Trastuzumab-[MC-VC-PAB-MMAE]₂ conjugate is similar to the preparation of ADC of example 34, but it adjusts the incubation temperature or time in step (1) as follows:

| Example | The incubation temperature (° C.) | The incubation time (h) |
|---|---|---|
| 102 | 15 | 4 |
| 103 | 24 | 4 |
| 104 | 37 | 4 |
| 105 | 4 | 0.25 |
| 106 | 4 | 0.5 |
| 106 | 4 | 0.75 |
| 108 | 4 | 1 |
| 109 | 4 | 2 |
| 110 | 4 | 3 |
| 111 | 4 | 4 |
| 112 | 4 | 5 |
| 113 | 4 | 6 |

### Example 114: Preparation of Trastuzumab-[MC-VC-PAB-MMAE]₂ Conjugate by the Engineered Antibody

The preparation of Trastuzumab-[MC-VC-PAB-MMAE]₂ conjugate is similar to the preparation of ADC of example 34, but it used the engineered antibody.

The engineered antibody is the mutant of trastuzumab by replacing disulfide bonds in-between heavy-light chain through cysteine to serine mutation (Order from Biointron).

### Example 115: Preparation of Trastuzumab-[Maleimide-PEG4-N3-DBCO-Cy3]1 (the ADC with D1)

### 1. Synthesis of Dibromomaleimide-PEG4-N3

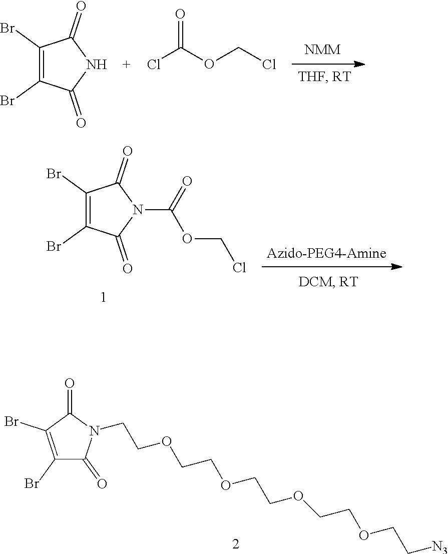 [SMILES: O=C1NC(=O)C(Br)=C1Br ; O=C(Cl)OCCl ; O=C(OCCl)N1C(=O)C(Br)=C(Br)C1=O ; [N-]=[N+]=NCCOCCOCCOCCOCCN1C(=O)C(Br)=C(Br)C1=O]

To a solution of 3, 4-dibromomaleimide (127 mg, 0.5 mmol, 1 eq) and N-methylmorpholine (0.22 mL, 2 mmol, 4 eq) in THF (3.5 mL), chloromethyl chloroformate (0.18 mL, 2 mmol, 4 eq) was added and the mixture was stirred for 20 min at room temperature. Then DCM (10 mL) was added, the organic phase was washed with H₂O, dried over MgSO₄ and the solvent removed in vacuo to yield the title product 1 (139 mg, 0.4 mmol, 80%).

A solution of Azido-PEG4-Amine (105 mg, 0.4 mmol, 1 eq, Xi'an Confluore Biological Technology Co., Ltd) in dichloromethane (2 mL) was added to a stirred solution of product 1 (139 mg, 0.4 mmol, 1 eq) in dichloromethane (2 mL).

After 30 minutes, dichloromethane (6 mL) was added and the solution washed with a 0.68 M acetate buffer pH 5 (10 mL), water (1 mL), and dried with MgSO₄. Concentration in vacuo followed by purification by column chromatography (100% EtOAc as the mobile phase) yielded dibromomaleimide-PEG4-N3 (the title product 2) as a pale yellow oil (150 mg, 0.3 mmol, 75%).

### 2. Preparation of Trastuzumab-[Maleimide-PEG4-N3-DBCO-Cy3]1

(1) incubating the first reductant TCEP-NO (0.02 mM) and trastuzumab (0.012 mM) in the presence of an effective amount of ZnCl₂ (0.24 mM) in BES (20 mM, pH7.0), The incubation temperature is 4° C. and the incubation time is 4 h;
(2) introducing EDTA (0.6 mM) and dibromomaleimide-PEG4-N3 (0.013 mM) to react with reduced thiol groups resulted from step (1), the reaction temperature is 24° C. and the reaction time is 3 h, then recovering the product using a desalting column to afford Trastuzumab-[Maleimide-PEG4-N3]₁;
(3) incubating Trastuzumab-Maleimide-PEG4-N3 and DBCO-Cy3 (0.02 mM) in MES (20 mM, pH6.7), the reaction temperature is 25° C. and the reaction time is 8 h, then recovering Trastuzumab-Maleimide-PEG4-N3-DBCO-Cy3 using a desalting column.

### Example 116: Preparation of Trastuzumab-[Maleimide]₁ [MC-GGFG-DXd]₆ (the ADC with D0+D6)

(1) incubating the first reductant TCEP-NO (0.02 mM) and trastuzumab (0.012 mM) in the presence of an effective amount of ZnCl₂ (0.24 mM) in MES (20 mM, pH6.7), The incubation temperature is 4° C. and the incubation time is 4 h;
(2) introducing EDTA (0.6 mM) and dibromomaleimide (0.013 mM) to react with reduced thiol groups resulted from step (1), the reaction temperature is 24° C. and the reaction time is 3 h, then recovering the product using a desalting column to afford Trastuzumab-[Maleimide]₁;
(3) introducing Trastuzumab-[Maleimide]₁ and TCEP (0.08 mM) in MES (20 mM, pH6.7), the reaction temperature is 25° C. and the reaction time is 12 h;
(4) introducing MC-GGFG-DXd (0.14 mM) to solution from step (3), and the reaction mixture was allowed to stay at 24° C. for 1 h, then recovering Trastuzumab-[Maleimide]₁ [MC-GGFG-DXd]₆ using a desalting column.

### Example 117: Preparation of Trastuzumab-[MC-MMAF]2 [MC-GGFG-DXd]₆ (the ADC with D2+D6)

(1) incubating the first reductant TCEP-NO prepared by example 1 (0.02 mM) and trastuzumab (0.012 mM) in the presence of an effective amount of ZnCl₂ (0.24 mM) in MES (20 mM, pH6.7), The incubation temperature is 4° C. and the incubation time is 4 h;
(2) introducing EDTA (0.6 mM) and an excess amount of MC-MMAF (0.06 mM) to react with reduced thiol groups resulted from step (1), the reaction temperature is 24° C. and the reaction time is 1 h, then recovering the product using a desalting column to afford Trastuzumab [MC-MMAF]2;
(3) introducing Trastuzumab-[MC-MMAF]2 and TCEP (0.08 mM) in MES (20 mM, pH6.7), the reaction temperature is 25° C. and the reaction time is 8 h;
(4) introducing MC-GGFG-DXd (0.14 mM) to solution from step (3), and the reaction mixture was allowed to stay at 24° C. for 1 h, then recovering the resultant Trastuzumab-[MC-MMAF]2 [MC-GGFG-DXd]₆ using a desalting column.

### Examples 118-119: Preparation of Trastuzumab-[MC-VC-PAB-MMAE]2 [MC-GGFG-DXd]₂ (the ADC with D2+D2)

(1) incubating the first reductant TCEP-NO (0.02 mM) and trastuzumab (0.012 mM) in the presence of an effective amount of ZnCl₂ (0.12 mM) in BES (20 mM, pH7.0), The incubation temperature is 4° C. and the incubation time is 4 h;
(2) introducing EDTA (0.6 mM) and MC-VC-PAB-MMAE (0.048 mM) to react with reduced thiol groups resulted from step (1), the reaction temperature is 24° C. and the reaction time is 1 h, then recovering the product using a desalting column to afford Trastuzumab-[MC-VC-PAB-MMAE]2;
(3) incubating ZnCl₂ (1.2 mM), the second reductant TCEP-3 (0.0144 mM)/TCEP-6 (0.0216 mM) and the product from step (2) in BES buffer (pH7.0, 20 mM) and the reaction mixture was allowed to stay at 4° C. for 4 h;
(4) introducing EDTA (3 mM) to trap Zn²⁺, and introducing MC-GGFG-DXd (0.1 mM) to react with the reduced thiol groups resulted from step (3), the reaction temperature is 24° C. and the reaction time is 1 h;
(5) the reaction mixture was subjected to purification using a desalting column.

### Example 120: Preparation of Trastuzumab-[Maleimide-PEG4-N3-DBCO-Cy3]1 [MC-VC-PAB-MMAE]₂ (the ADC with D1+D2)

(1) incubating the first reductant TCEP-NO (0.02 mM) and trastuzumab (0.012 mM) in the presence of an effective amount of ZnCl₂ (0.24 mM) in BES (20 mM, pH7.0), The incubation temperature is 4° C. and the incubation time is 4 h;
(2) introducing EDTA (0.6 mM) and dibromomaleimide-PEG4-N3 (0.013 mM) to react with reduced thiol groups resulted from step (1), the reaction temperature is 25° C. and the reaction time is 1 h, then recovering the product using a desalting column to afford Trastuzumab-[Maleimide-PEG4-N3]1;
(3) incubating Trastuzumab-[Maleimide-PEG4-N3]₁ from step (2) and DBCO-Cy3 (0.02 mM) in MES (20 mM, pH6.7), the reaction temperature is 25° C. and the reaction time is 6 h, then recovering Trastuzumab-[Maleimide-PEG4-N3-DBCO-Cy3]₁ using a desalting column.
(4) incubating ZnCl₂ (1.2 mM), the second reductant TCEP-3 (0.0144 mM) and the product prepared from step (3) in BES buffer (pH7.0, 20 mM) and the reaction mixture was allowed to stay at 4° C. for 4 h;
(5) introducing EDTA (3 mM) to trap Zn²⁺, and introducing MC-VC-PAB-MMAE (0.1 mM) to react with the reduced thiol groups resulted from step (4), the reaction temperature is 25° C. and the reaction time is 1 h;
(6) the reaction mixture was subjected to purification using a desalting column.

### Examples 121-122: Preparation of Trastuzumab-[Maleimide]₁ [MC-VC-PAB-MMAE]₄ (the ADC with D0+D4)

(1) incubating the first reductant TCEP-NO (0.02 mM) and trastuzumab (0.012 mM) in the presence of an effective amount of ZnCl₂ (0.24 mM) in MES (20 mM, pH6.7), The incubation temperature is 4° C. and the incubation time is 4 h;
(2) introducing EDTA (0.6 mM) and dibromomaleimide (0.013 mM) to react with reduced thiol groups resulted from step (1), the reaction temperature is 24° C. and the reaction time is 3 h, then recovering the product using a desalting column to afford Trastuzumab-Maleimide;
(3) incubating ZnCl₂ (0.36 mM), the second reductant TCEP-3 (0.036 mM) or TCEP-6 (0.048 mM) and the product from step (2) in BES buffer (pH7.0, 20 mM) and the reaction mixture was allowed to stay at 4° C. for 16 h;
(4) introducing EDTA (0.6 mM) to trap Zn²⁺, and introducing MC-VC-PAB-MMAE (0.1 mM) to react with the reduced thiol groups resulted from step (4), the reaction temperature is 24° C. and the reaction time is 1 h;
(5) the reaction mixture was subjected to purification using a desalting column.

### Examples 123-124: Preparation of Trastuzumab-[MC-GGFG-DXd]2 [MC-VC-PAB-MMAE]₄ (the ADC with D2+D4)

(1) incubating the first reductant TCEP-NO (0.02 mM) and trastuzumab (0.012 mM) in the presence of an effective amount of ZnCl₂ (0.24 mM) in BES (20 mM, pH7.0), The incubation temperature is 4° C. and the incubation time is 4 h;
(2) introducing EDTA (0.6 mM) and MC-GGFG-DXd (0.072 mM) to react with reduced thiol groups resulted from step (1), the reaction temperature is 24° C. and the reaction time is 1 h, then recovering the product using a desalting column to afford Trastuzumab-[MC-GGFG-DXd]2;
(3) incubating ZnCl₂ (0.36 mM), the second reductant TCEP-3 (0.036 mM) or TCEP-6 (0.048 mM) and the product from step (2) in BES buffer (pH7.0, 20 mM) and the reaction mixture was allowed to stay at 4° C. for 16 h;
(4) introducing EDTA (0.6 mM) to trap Zn²⁺, and introducing MC-VC-PAB-MMAE (0.1 mM) to react with the reduced thiol groups resulted from step (4), the reaction temperature is 24° C. and the reaction time is 1 h;
(5) the reaction mixture was subjected to purification using a desalting column.

### Example 125: Preparation of Trastuzumab-[Maleimide-PEG4-N3-DBCO-Cy3]1 [MC-VC-PAB-MMAE]₄ (the ADC with D1+D4)

(1) incubating the first reductant TCEP-NO (0.02 mM) and trastuzumab (0.012 mM) in the presence of an effective amount of ZnCl₂ (0.24 mM) in BES (20 mM, pH7.0), The incubation temperature is 4° C. and the incubation time is 4 h;
(2) introducing EDTA (0.6 mM) and dibromomaleimide-PEG4-N3 (0.013 mM) to react with reduced thiol groups resulted from step (1), the reaction temperature is 25° C. and the reaction time is 6 h, then recovering the product using a desalting column to afford Trastuzumab-Maleimide-PEG4-N3;
(3) incubating Trastuzumab-Maleimide-PEG4-N3 and DBCO-Cy3 (0.02 mM) in MES (20 mM, pH6.7), the reaction temperature is 25° C. and the reaction time is 6 h, then recovering Trastuzumab-Maleimide-PEG4-N3-DBCO-Cy3 using a desalting column.
(4) incubating ZnCl₂ (0.36 mM), the second reductant TCEP-3 (0.0408 mM) and the product from step (3) in BES buffer (pH7.0, 20 mM) and the reaction mixture was allowed to stay at 4° C. for 16 h;
(5) introducing EDTA (0.6 mM) to trap Zn²⁺, and introducing MC-VC-PAB-MMAE (0.1 mM) to react with the reduced thiol groups resulted from step (4), the reaction temperature is 24° C. and the reaction time is 1 h;
(6) the reaction mixture was subjected to purification using a desalting column.

Comparative Examples 1-9: Preparation of ADCs without the transition metal ions ADCs with D2 were prepared as follows:
(1) TCEP-NO, TCEP-3NO or TCEP-CO (0.02 mM) was added to a solution of a monoclonal antibody (0.012 mM, in MES buffer, pH6.7, 20 mM) and the reaction mixture was allowed to stay at 4° C. for 4 h, 8 h, or 12 h, respectively;
(2) MC-VC-PAB-MMAE (0.06 mM) in DMA was introduced and the reaction was continued at 24° C. for 1 h;
(3) Cysteine (0.08 mM) was added to deplete excessive MC-VC-PAB-MMAE;
(4) The reaction mixture was subjected to purification using a de-salting column.

The monoclonal antibodies and reductants used are as follows:

| Comparative | Serial | | | |
|---|---|---|---|---|
| Example | NO. | ADC | Antibody | Reductant |
| 1 | 381 | Trastuzumab-MC-VC-PAB-MMAE | Trastuzumab | TCEP-NO |
| 2 | 683 | Sacituzumab-MC-VC-PAB-MMAE | Sacituzumab | TCEP-NO |
| 3 | 684 | Belantamab-MC-VC-PAB-MMAE | Belantamab | TCEP-NO |
| 4 | 693 | Trastuzumab-MC-VC-PAB-MMAE | Trastuzumab | TCEP-3NO |
| 5 | 677 | Sacituzumab-MC-VC-PAB-MMAE | Sacituzumab | TCEP-3NO |
| 6 | 679 | Belantamab-MC-VC-PAB-MMAE | Belantamab | TCEP-3NO |
| 7 | 378 | Trastuzumab-MC-VC-PAB-MMAE | Trastuzumab | TCEP-CO |
| 8 | 673 | Sacituzumab-MC-VC-PAB-MMAE | Sacituzumab | TCEP-CO |
| 9 | 675 | Belantamab-MC-VC-PAB-MMAE | Belantamab | TCEP-CO |

### Comparative Example 11: Preparation of ADC with TCEP

(1) TCEP (0.02 mM) was added to a solution of Transtuzumab (0.012 mM, in MES buffer, pH6.7, 20 mM) and the reaction mixture was allowed to stay at 4° C. for 4 h;
(2) MC-VC-PAB-MMAE (0.06 mM) in DMA was introduced and the reaction was continued at 24° C. for 30 min;
(3) cysteine (0.08 mM) was added to deplete excessive MC-VC-PAB-MMAE;
(4) The reaction mixture was subjected to purification using a de-salting column.

### Comparative Example 12: Preparation of ADC with TCEP

(1) ZnCl2 (0.24 mM) and TCEP (0.02 mM) were added to a solution of Transtuzumab (0.012 mM, in MES buffer, pH6.7, 20 mM) and the reaction mixture was allowed to stay at 4° C. for 4 h;
(2) EDTA (0.6 mM) was added to trap Zn²⁺;
(3) MC-VC-PAB-MMAE (0.06 mM) in DMA was introduced and the reaction was continued at 24° C. for 30 min;
(4) cysteine (0.08 mM) was added to deplete excessive MC-VC-PAB-MMAE;
(5) The reaction mixture was subjected to purification using a de-salting column.

### Homogeneity Assays

The drug/antibody ratio (DAR) and product distribution were analyzed using HIC-HPLC (Agilent1200) with a TSK gel Butyl-NPR column (4.6 mm IDX 3.5 cm) (commercially available from Tosoh Biosciences) at a flow rate of 0.5 mL/min at 30° C. Solvent A was 1.5 M (NH₄)₂SO₄ and 50 mM potassium phosphate pH 7. Solvent B was 75% v/v 50 mM potassium phosphate pH 7 and 25% v/v isopropanol. The washout procedure is as follows:

| Time [min] | Solvent A [%] | Solvent B [%] |
|---|---|---|
| 0.00 | 100.0 | 0.0 |
| 2.00 | 100.0 | 0.0 |
| 15.00 | 0.0 | 100.0 |
| 17.00 | 0.0 | 100.0 |
| 18.00 | 100.0 | 0.0 |
| 20.00 | 100.0 | 0.0 |

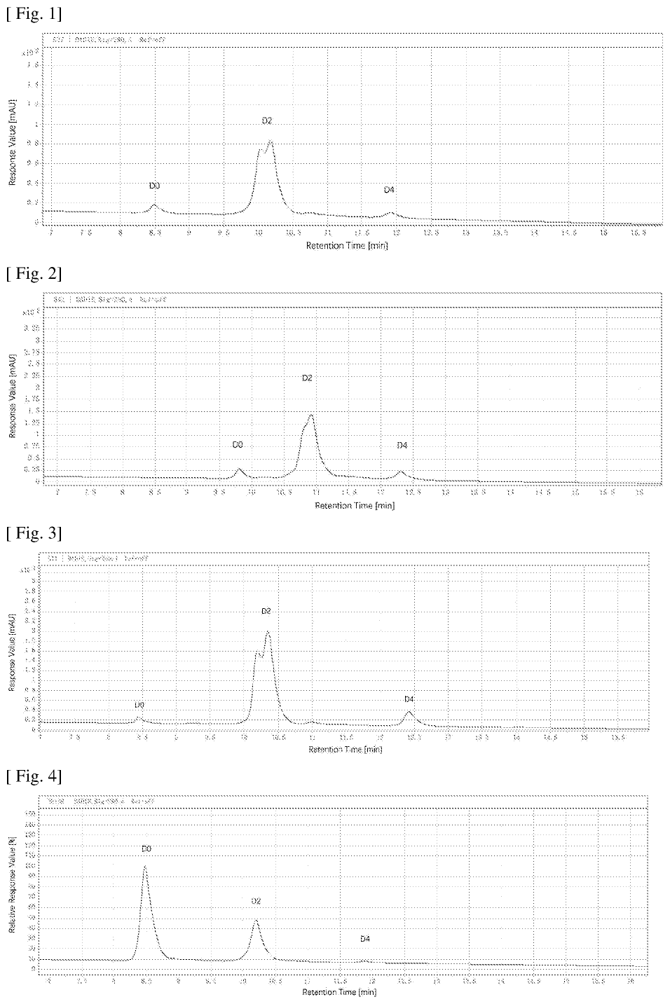

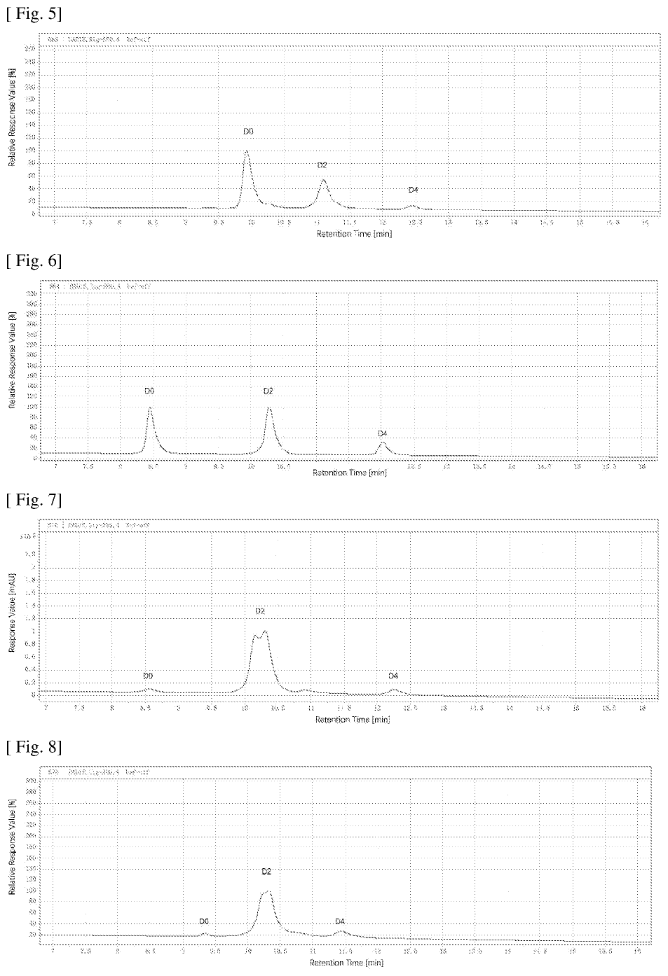

The results of Examples 34-36 and Comparative Examples 1-3 are shown in Table 1. The chromatograms are shown in FIGS. 1-6.

As the results shown in table 1, ADCs of Examples 34-36 and comparative examples 1-3 prepared by TCEP-NO obtain different D2 and D4 ratios, which indicates MC—VC-PAB-MMAE is successfully linked to Trastuzumab, Sacituzumab or Belantamab. TCEP-NO could be used as a reductant in antibody modification and preparation of ADC. In contrast to C1-C3, ADCs of Examples 34-36 prepared by TCEP-NO significantly increase D2 ratio. This indicates TCEP-NO has the reduction selectivity in the presence of Zn²⁺, TCEP-NO could be used to prepare the ADC with D2.

TABLE 1

| | | D0 | D2 | D4 | D6 | D8 |
|---|---|---|---|---|---|---|
| Example | Figure | (wt %) | (wt %) | (wt %) | (wt %) | (wt %) |
| 34 | 1 | 4.55 | 91.09 | 4.36 | 0 | 0 |
| 35 | 2 | 5.98 | 84.97 | 9.06 | 0 | 0 |
| 36 | 3 | 1.77 | 89.2 | 9.04 | 0 | 0 |
| C1 | 4 | 69.85 | 30.15 | 0 | 0 | 0 |
| C2 | 5 | 58.11 | 37.6 | 4.29 | 0 | 0 |
| C3 | 6 | 38.79 | 48.82 | 12.39 | 0 | 0 |

“E” was short for Example. “C” was short for Comparative example.

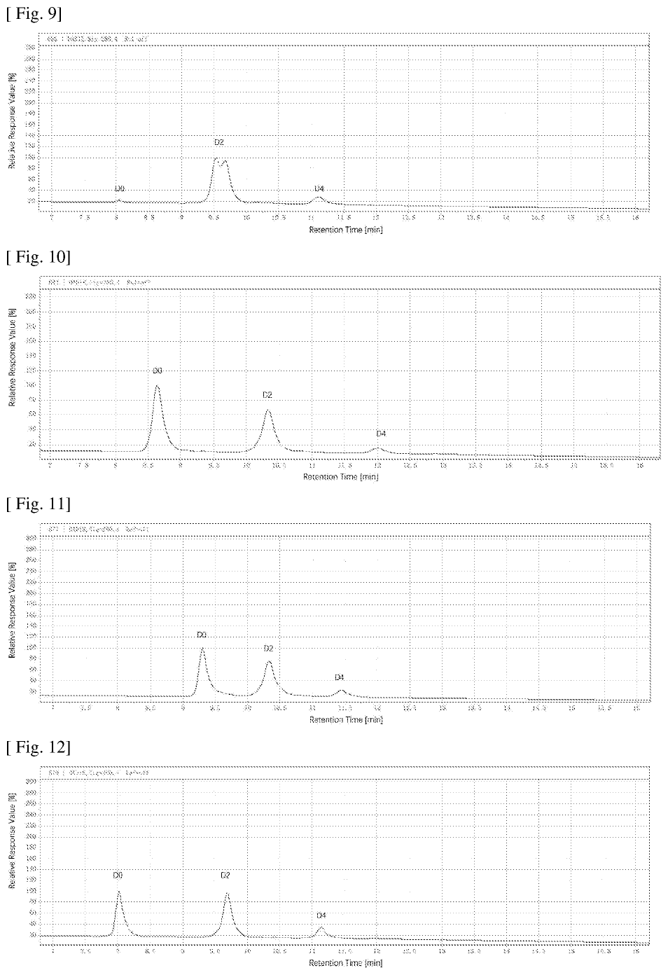

The results of Examples 37-39 and Comparative Examples 4-6 are shown in Table 2, and the chromatograms are shown in FIGS. 7-12.

As the results shown in table 2, ADCs of Examples 37-39 and C4-C6 prepared by TCEP-3NO obtain different D2 and D4 ratios, which indicates MC-VC-PAB-MMAE is successfully linked to Trastuzumab, Sacituzumab or Belantamab. TCEP-3NO could be used as a reductant in antibody modification and preparation of ADC. In contrast to C4-C6, ADCs of Examples 37-39 prepared by TCEP-3NO significantly increase D2 ratio. This indicates TCEP-3NO has the reduction selectivity in the presence of Zn²⁺, TCEP-3NO could be used to prepare the ADC with D2.

TABLE 2

| | | D0 | D2 | D4 | D6 | D8 |
|---|---|---|---|---|---|---|
| Example | Figure | (wt %) | (wt %) | (wt %) | (wt %) | (wt %) |
| 37 | 7 | 2.87 | 91.47 | 5.67 | 0 | 0 |
| 38 | 8 | 2.85 | 87.86 | 9.29 | 0 | 0 |
| 39 | 9 | 1.52 | 87.62 | 10.86 | 0 | 0 |
| C4 | 10 | 52.13 | 42.34 | 5.53 | 0 | 0 |
| C5 | 11 | 45.05 | 45.92 | 9.04 | 0 | 0 |
| C6 | 12 | 38.56 | 49.01 | 12.43 | 0 | 0 |

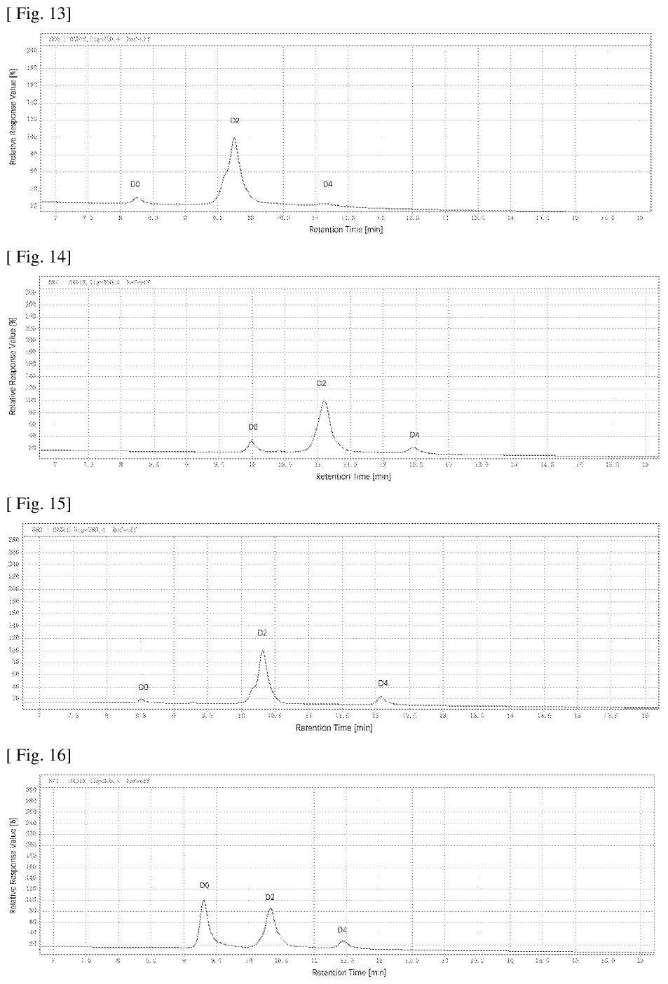

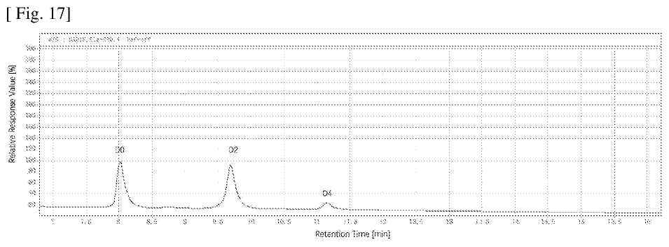

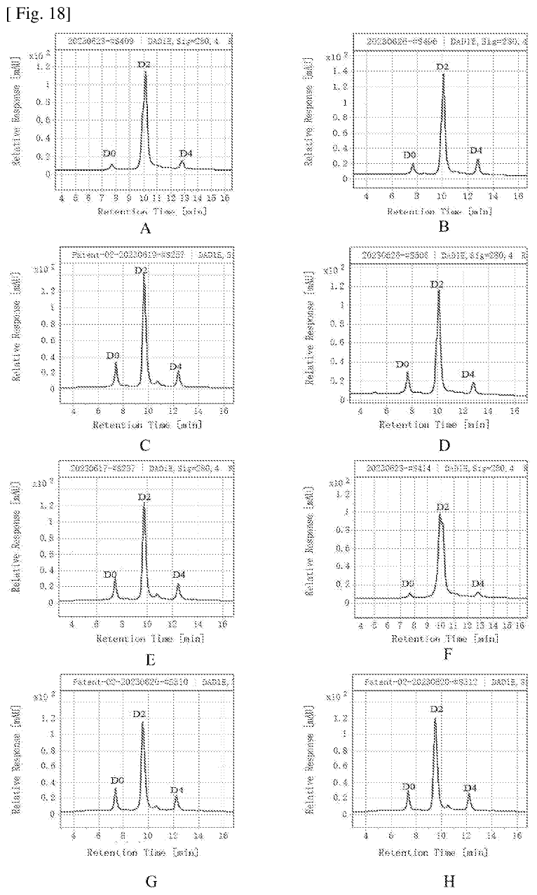

The results of Examples 40-42 and Comparative Examples 7-9 are shown in Table 3, and the chromatograms are shown in FIG. 13-17.

As the results shown in table 3, ADCs of Examples 40-42 and C7-C9 prepared by TCEP-CO obtain different D2 and D4 ratios, which indicates MC-VC-PAB-MMAE is successfully connected to Trastuzumab, Sacituzumab or Belantamab. TCEP-CO could be used as a reductant in antibody modification and preparation of ADC. In contrast to C7-C9, ADCs of Examples 40-42 prepared by TCEP-CO significantly increase D2 ratio. This indicates TCEP-CO has the reduction selectivity in the presence of Zn²⁺, TCEP-CO could be used to prepare the ADC with D2.

TABLE 3

| | | D0 | D2 | D4 | D6 | D8 |
|---|---|---|---|---|---|---|
| Example | Figure | (wt %) | (wt %) | (wt %) | (wt %) | (wt %) |
| 40 | 13 | 4.87 | 90.51 | 4.63 | 0 | 0 |
| 41 | 14 | 9.91 | 81.26 | 8.83 | 0 | 0 |
| 42 | 15 | 3.94 | 85.44 | 10.62 | 0 | 0 |
| C7 | — | — | — | — | — | — |
| C8 | 16 | 42.92 | 48.2 | 8.88 | 0 | 0 |
| C9 | 17 | 42.54 | 50 | 7.46 | 0 | 0 |

With a conjugation process using the same steps without the addition of transition metal ions in step (a) as a negative control (see table 1-3), the disclosure successfully demonstrated that combination of transition metal ions and novel reductants is responsible for higher level of D2 in the resultant ADCs. Furthermore, it confirmed this new process generates ADC products with a high Fc and/or Fab, preference. By using the process of the present disclosure to produce antibody-drug conjugates, the homogeneity of the antibody-drug conjugates is dramatically higher.

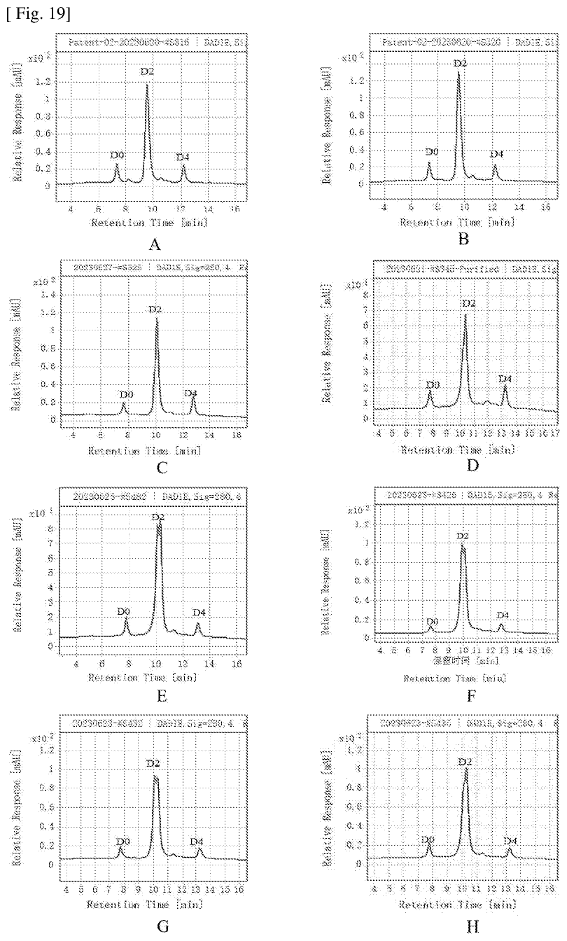

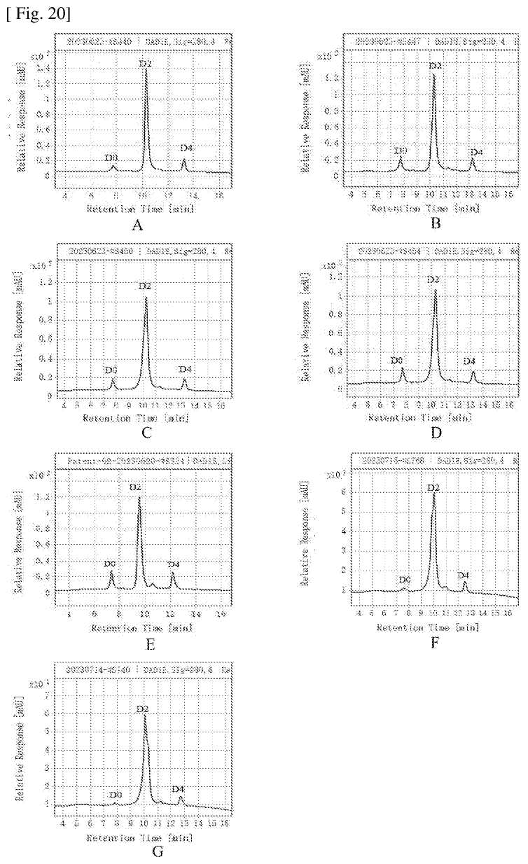

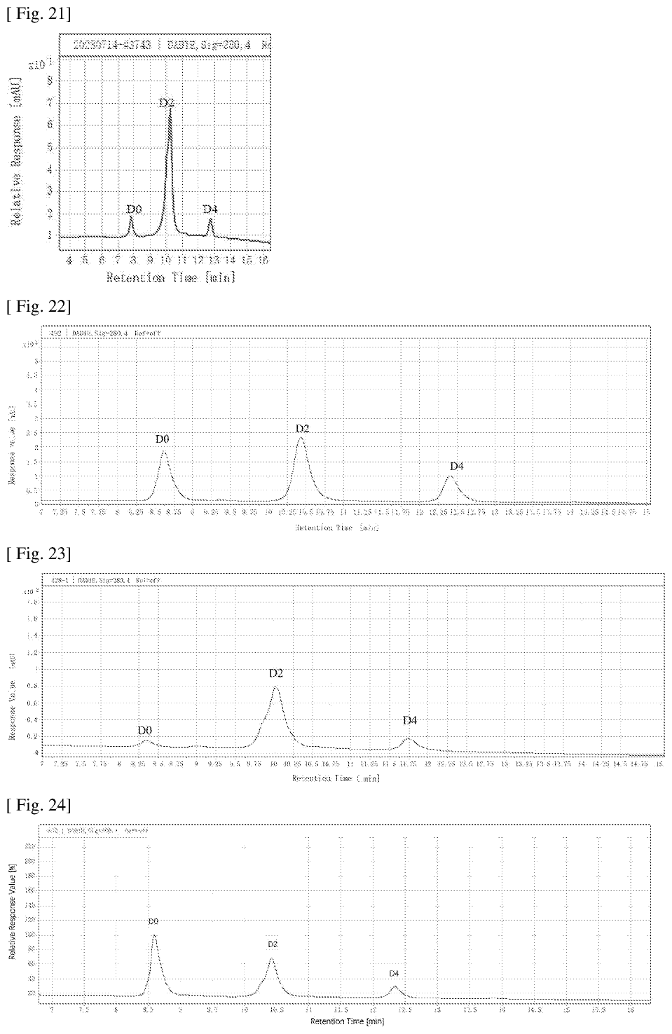

As the results of examples 43-66 and comparative examples 11-12 shown in table 4 and the FIGS. 18-23, the compounds in the present application could increase the homogeneity of the ADC with D2 compared with the traditional method using TCEP without Zn²⁺, wherein, the selective reduction ability of TCEO-6 is best, with a D2 content of up to 94.25%. Meanwhile, the selective reduction ability of TCEP-NO, TCEP-3NO, TCEP-CO, TCEP-1, TCEP-19, TCEP-20, TECP-21, TCEP-23, TCEP-24, TCEP-26, TCEP-28, TCEP-34, TCEP-35 and TCEP-37 is also wonderful, with a D2 content of up to 84%, 87%, even to 90% or 93%.

TABLE 4

| | | | D0 | D2 | D4 |
|---|---|---|---|---|---|
| Example | Figure | Reductant | (wt %) | (wt %) | (wt %) |
| 43 | 18A | TCEP-1 | 3.47 | 90.75 | 5.78 |
| 44 | 18B | TCEP-2 | 6.73 | 83.46 | 9.82 |
| 45 | 18C | TCEP-3 | 11.83 | 80.00 | 8.17 |
| 46 | 18D | TCEP-4 | 11.83 | 81.08 | 7.09 |
| 47 | 18E | TCEP-5 | 11.82 | 77.63 | 10.56 |
| 48 | 18F | TCEP-6 | 2.67 | 94.25 | 3.08 |
| 49 | 18G | TCEP-7 | 13.08 | 76.81 | 10.11 |
| 50 | 18H | TCEP-8 | 10.37 | 78.93 | 10.70 |
| 51 | 19A | TCEP-9 | 9.95 | 78.49 | 11.56 |
| 52 | 19B | TCEP-10 | 8.99 | 82.27 | 8.74 |
| 53 | 19C | TCEP-15 | 7.80 | 79.66 | 12.54 |
| 54 | 19D | TCEP18 | 10.27 | 77.66 | 12.07 |
| 55 | 19E | TCEP19 | 6.66 | 88.03 | 5.31 |
| 56 | 19F | TCEP20 | 3.87 | 91.57 | 4.55 |
| 57 | 19G | TCEP21 | 6.22 | 87.28 | 6.50 |
| 58 | 19H | TCEP23 | 7.10 | 87.62 | 5.28 |
| 59 | 20A | TCEP24 | 5.15 | 85.25 | 9.60 |
| 60 | 20B | TCEP25 | 10.14 | 81.34 | 8.52 |
| 61 | 20C | TCEP26 | 6.24 | 86.89 | 6.87 |
| 62 | 20D | TCEP28 | 8.23 | 84.97 | 6.80 |
| 63 | 20E | TCEPA | 10.11 | 79.17 | 10.71 |
| 64 | 20F | TCEP34 | 2.68 | 91.99 | 5.33 |
| 65 | 20G | TCEP35 | 1.69 | 93.05 | 5.26 |
| 66 | 21 | TCEP37 | 8.75 | 84.20 | 7.05 |
| C11 | 22 | TCEP without | 32.5 | 48.98 | 18.52 |
| | | Zn²⁺ | | | |
| C12 | 23 | TCEP with | 5.22 | 80.6 | 14.17 |
| | | Zn²⁺ | | | |

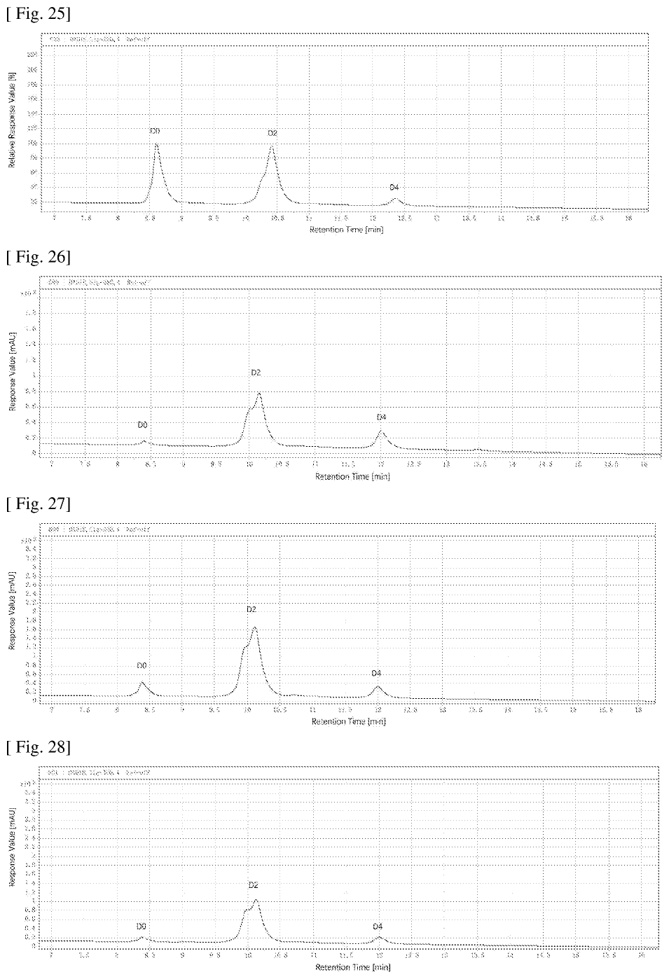

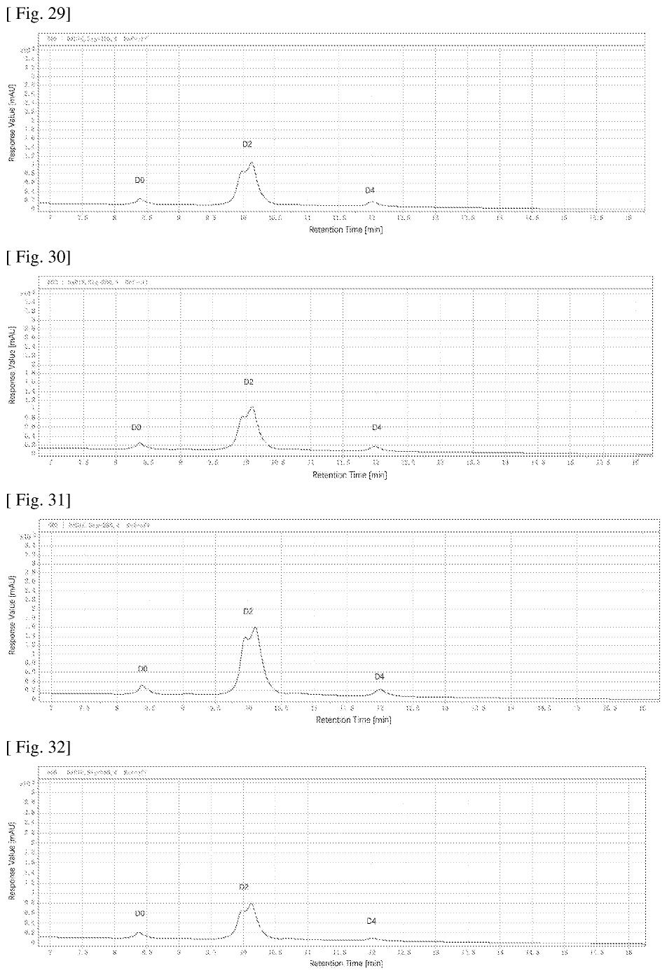

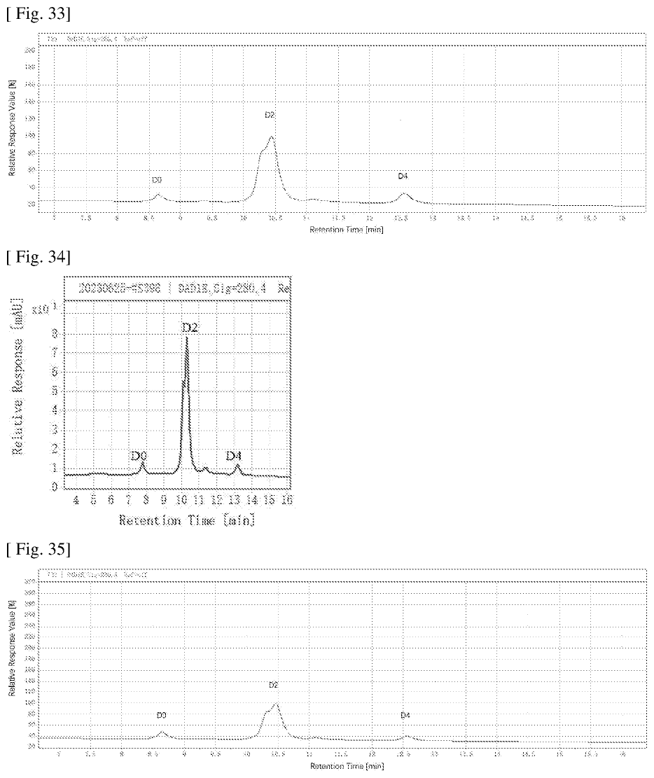

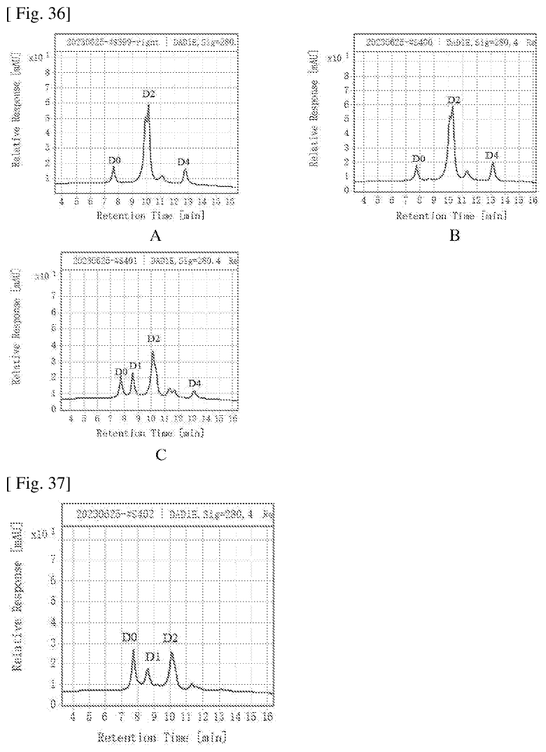

The results of Examples 67-81 and comparative examples 10 are shown in Table 5, and the chromatograms are shown in FIGS. 24-37. As the results shown in the table 5, by adding the transition metal ions, the content of D2 increases. D2 ratio increases as Zn²⁺/TCEP-NO molar ratio increases from 0.4 to 6. After that, D2 ratio reaches a plateau. when the molar ratio of Zn²⁺/TCEP-NO is up to 200:1 and 250:1, the content of D2 is lower than that of Zn²⁺/TCEP-NO molar ratio ranging from 2:1 to 125:1. This indicates the transition metal ions, especially the Zn²⁺/TCEP-NO ratio, plays a key role in determining the D2 ratio and the reduction selectivity.

TABLE 5

| | | Zn/TCEP-NO | D0 | D1 | D2 | D4 | D6 | D8 |
|---|---|---|---|---|---|---|---|---|
| Example | Figure | (Molar Ratio) | (wt %) | (wt %) | (wt %) | (wt %) | (wt %) | (wt %) |
| C1 | 4 | 0 | 69.85 | 0 | 30.15 | 0 | 0 | 0 |
| E67 | 24 | 0.4 | 48.78 | 0 | 41.35 | 9.87 | 0 | 0 |
| E68 | 25 | 1 | 39.19 | 0 | 55.53 | 5.28 | 0 | 0 |
| E69 | 26 | 2 | 3.15 | 0 | 78.12 | 18.74 | 0 | 0 |
| E70 | 27 | 4 | 8.57 | 0 | 82.55 | 8.88 | 0 | 0 |
| E71 | 28 | 6 | 4.19 | 0 | 87.32 | 8.48 | 0 | 0 |
| E72 | 29 | 8 | 5.16 | 0 | 88.52 | 6.33 | 0 | 0 |
| E73 | 30 | 10 | 6.09 | 0 | 87.83 | 6.08 | 0 | 0 |
| E74 | 31 | 14 | 5.45 | 0 | 89.64 | 4.91 | 0 | 0 |
| E75 | 32 | 16 | 6.37 | 0 | 89.59 | 4.04 | 0 | 0 |
| E76 | 33 | 24 | 4.44 | 0 | 87.03 | 8.53 | 0 | 0 |
| E77 | 34 | 30 | 4.99 | 0 | 90.38 | 4.63 | 0 | 0 |
| E78 | 35 | 60 | 8.91 | 0 | 86.52 | 4.57 | 0 | 0 |
| E79 | 36A | 70 | 8.29 | 0 | 82.00 | 9.71 | 0 | 0 |
| E80 | 36B | 125 | 7.47 | 0 | 81.22 | 11.32 | 0 | 0 |
| E81 | 36C | 200 | 14.31 | 15.11 | 63.75 | 6.83 | 0 | 0 |
| C10 | 37 | 250 | 32.03 | 14.64 | 53.33 | 0 | 0 | 0 |

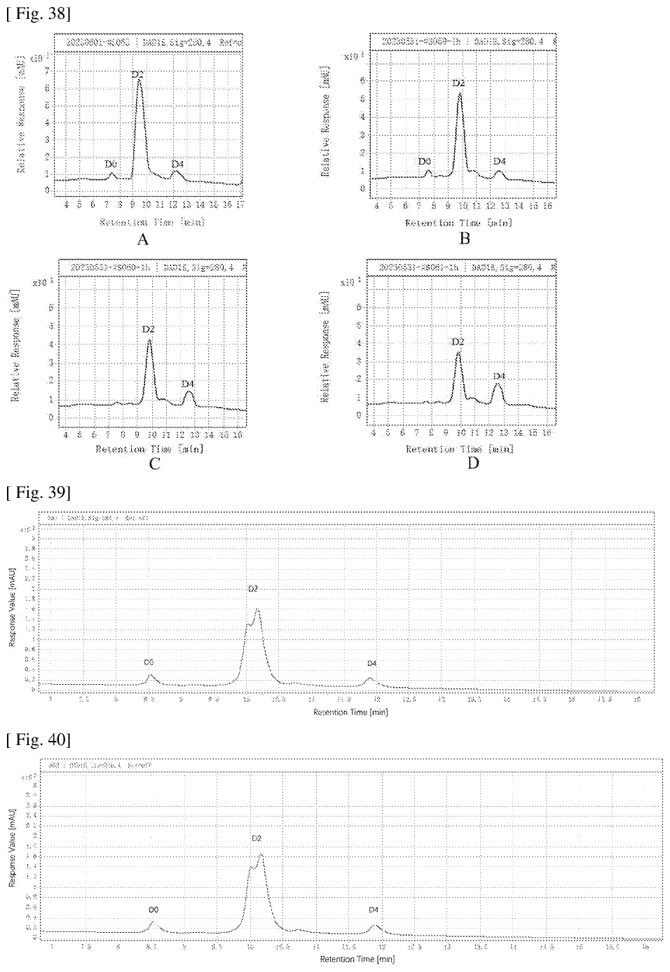

The results of Examples 82-85 are shown in Table 6, and the chromatograms are shown in FIG. 38. As the results shown in the table 6 and examples 46, 49, 53 and 54, when the molar ratio of antibody/TCEP-NO is 1:0.9 to 1:3.0, the content of the ADC with D2 is up to 55%, 60%, 70%, 75%, even to 80%, 85% or 90%. When the molar ratio of antibody/TCEP-NO is 1:2 and 1:2.5, the reduction time is shortened to 1 h and the content of D2 is greater than 80%.

TABLE 6

| | | Antibody/TCEP-NO | D0 | D2 | D4 |
|---|---|---|---|---|---|
| Example | Figure | (Molar Ratio) | (wt %) | (wt %) | (wt %) |
| 82 | 38A | 1:1.8 | 3.07 | 89.77 | 7.16 |
| 83 | 38B | 1:2.0 | 3.21 | 84.98 | 11.83 |
| 84 | 38C | 1:2.5 | 0 | 74.07 | 25.93 |
| 85 | 38D | 1:3.0 | 0 | 56.05 | 39.22 |

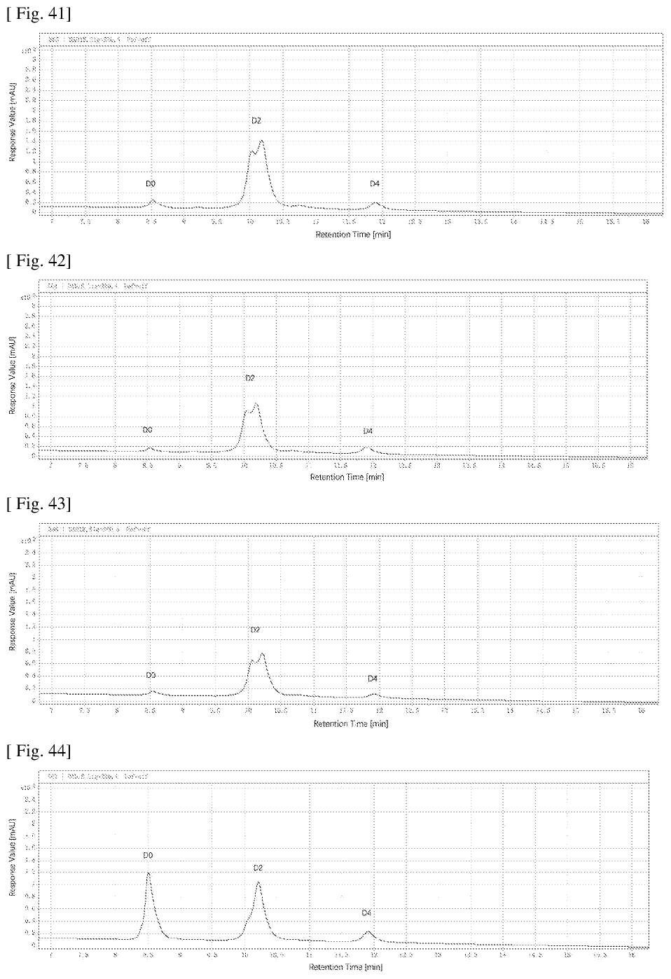

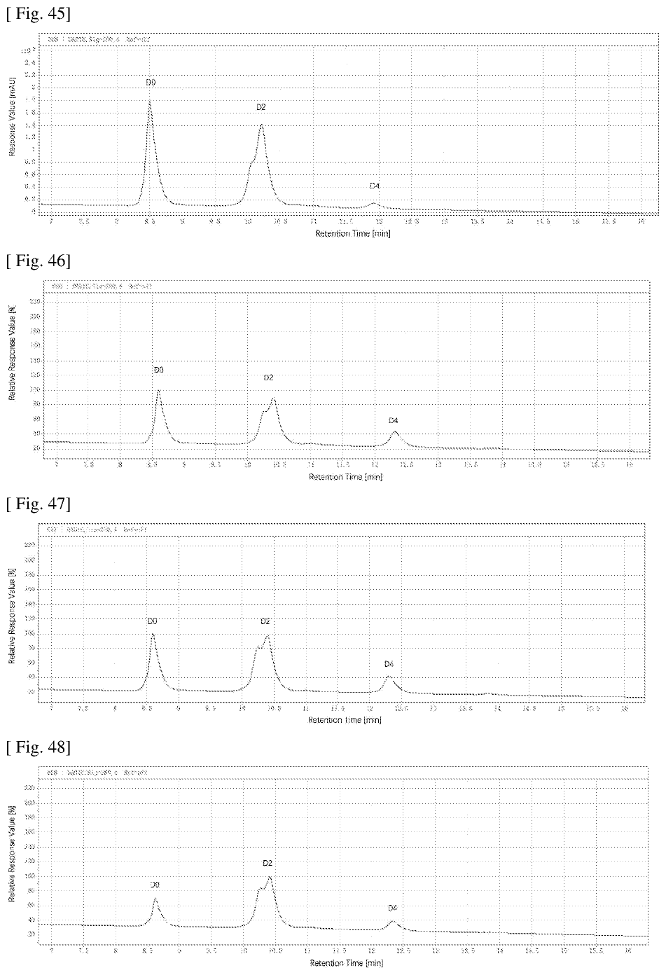

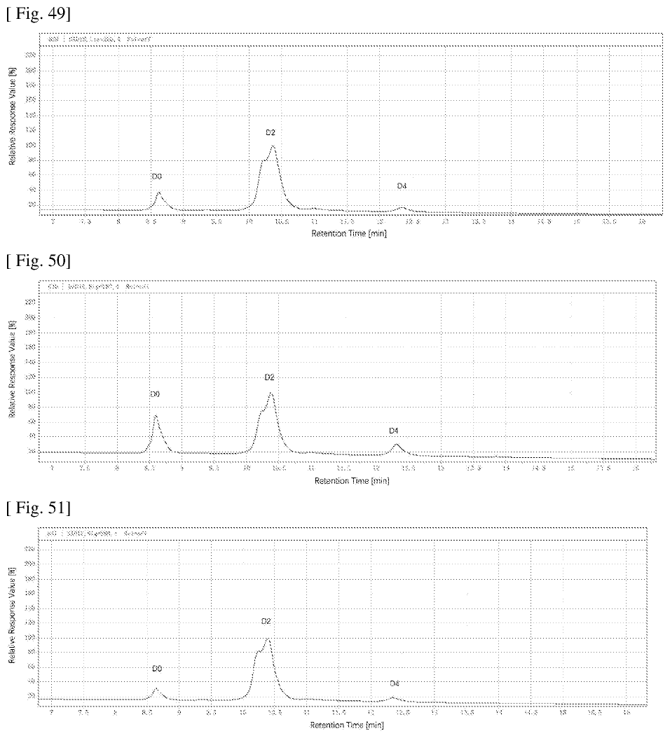

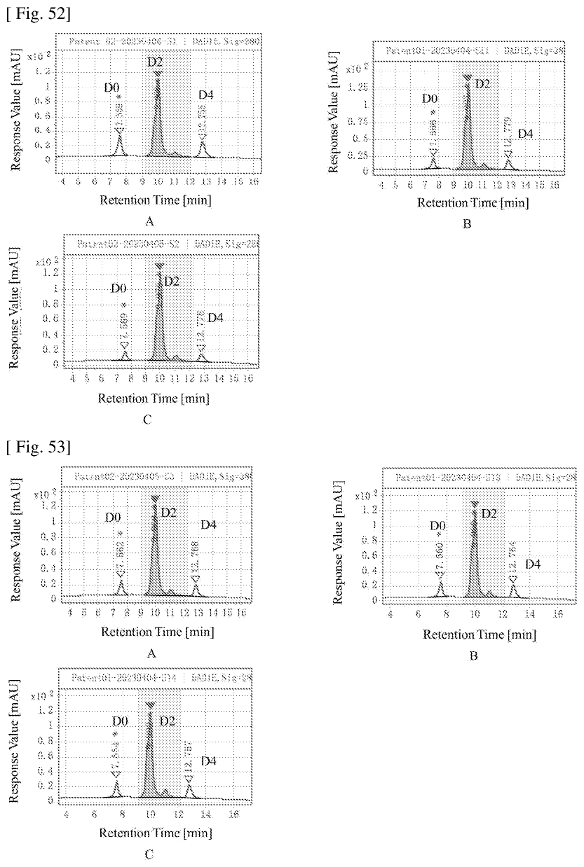

The results of Examples 86-101 are shown in Table 7, and the chromatograms are shown in FIGS. 39-52. As shown from the results in Table 7, the different buffers dramatically affect the reduction kinetics and selectivity. The buffer system in examples 86-101 are useful to improve the content of the ADC with D2.

TABLE 7

| | | | D0 | D2 | D4 | D6 | D8 |
|---|---|---|---|---|---|---|---|
| Example | Figure | Buffer | (wt %) | (wt %) | (wt %) | (wt %) | (wt %) |
| 86 | 39 | Bis-Tris buffer | 5.76 | 87.99 | 6.25 | 0 | 0 |
| 87 | 40 | PIPES buffer | 5.83 | 88.16 | 6.01 | 0 | 0 |
| 88 | 41 | MOPS buffer | 4.23 | 89.95 | 5.83 | 0 | 0 |
| 89 | 42 | BES buffer | 2.71 | 89.57 | 7.72 | 0 | 0 |
| 90 | 43 | HEPES buffer | 3.59 | 91.75 | 4.65 | 0 | 0 |
| 91 | 44 | ADA buffer | 39.6 | 51.8 | 8.6 | 0 | 0 |
| 92 | 45 | PB buffer | 42.99 | 54.23 | 2.77 | 0 | 0 |
| 93 | 46 | DIPSO buffer | 34.18 | 53.49 | 12.33 | 0 | 0 |
| 94 | 47 | MOBS buffer | 31.2 | 56.64 | 12.16 | 0 | 0 |
| 95 | 48 | MOPSO buffer | 19.71 | 70.93 | 9.36 | 0 | 0 |
| 96 | 49 | TES buffer | 11.6 | 84.7 | 3.7 | 0 | 0 |
| 97 | 50 | ACES buffer | 23.33 | 68.1 | 8.57 | 0 | 0 |
| 98 | 51 | TAPSO buffer | 7.98 | 88.41 | 3.62 | 0 | 0 |
| 99 | 52A | MES buffer | 13.40 | 74.51 | 12.09 | 0 | 0 |
| 100 | 52B | MES buffer | 7.14 | 85.48 | 7.38 | 0 | 0 |
| 101 | 52C | MES buffer | 6.18 | 87.57 | 6.25 | 0 | 0 |

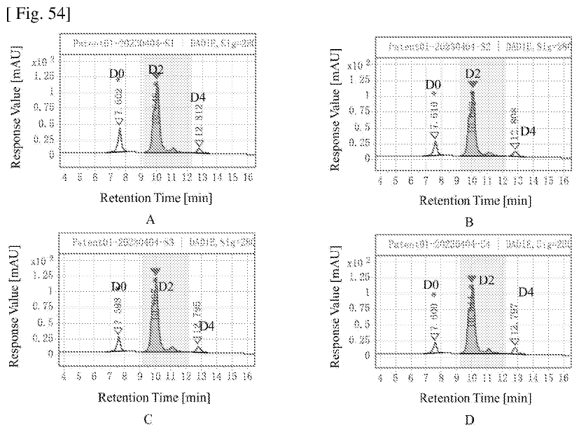

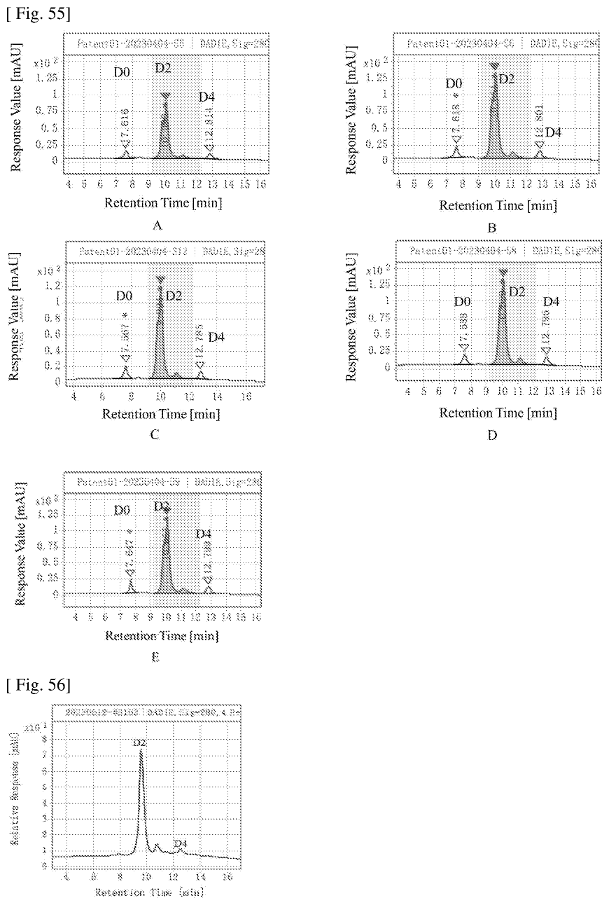

The results of Examples 102-113 are shown in Table 8, and the chromatograms are shown in FIGS. 53-55. As the results shown in the table 8, when the reduction temperature is 4-37° C. and the reduction time is 0.25 h to 6 h, the content of the ADC with D2 is up to 80%. the content of D2 increases as the reduction time of step (1) from 0.25 h to 1 h, and reaches plateau after 1 h, indicating a very fast reaction kinetics.

TABLE 8

| | | The | | | | |
|---|---|---|---|---|---|---|
| | | incubation | The | | | |
| | | temper- | incubation | | | |
| | | ature | time | D0 | D2 | D4 |
| Example | Figure | (° C.) | (h) | (wt %) | (wt %) | (wt %) |
| 102 | 53A | 15 | 4 | 8.88 | 83.16 | 7.96 |
| 103 | 53B | 24 | 4 | 9.32 | 81.24 | 9.45 |
| 104 | 53C | 37 | 4 | 9.29 | 80.73 | 9.98 |
| 105 | 54A | 4 | 0.25 | 15.98 | 80.25 | 3.77 |
| 106 | 54B | 4 | 0.5 | 10.89 | 84.77 | 4.34 |
| 107 | 54C | 4 | 0.75 | 9.63 | 85.52 | 4.85 |
| 108 | 54D | 4 | 1 | 7.86 | 86.76 | 5.38 |
| 109 | 55A | 4 | 2 | 7.36 | 87.41 | 5.23 |
| 110 | 55B | 4 | 3 | 6.89 | 87.46 | 5.66 |
| 111 | 55C | 4 | 4 | 7.41 | 87.29 | 5.30 |
| 112 | 55D | 4 | 5 | 6.57 | 87.86 | 5.58 |
| 113 | 55E | 4 | 6 | 6.54 | 87.19 | 6.27 |

The results of Example 114 are shown in Table 9, and the chromatograms are shown in FIG. 56. As the results shown in the table 9, the content of D2 prepared by the engineered antibody is as high as 96%. Those results indicated that this method is also applied to antibodies with simple mutations and might have even better reduction selectivity in some mutant antibodies.

TABLE 9

| | | D0 | D2 | D4 |
|---|---|---|---|---|
| Example | The engineered antibody | (wt %) | (wt %) | (wt %) |
| 114 | cysteine to serine mutation | 0 | 95.85 | 4.15 |

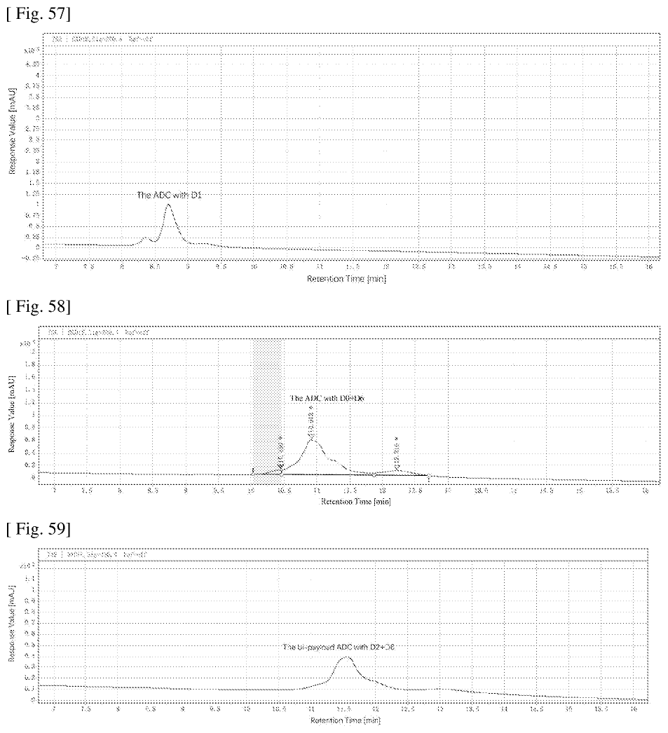

As shown in table 10, and FIG. 57, the results demonstrate that the content of the ADC with D1 is generally up to 83%. As shown in table 11, and FIG. 58, the results demonstrate that the content of the ADC with D0+D6 is generally up to 84.68%. As shown in table 12, and FIG. 59, the results demonstrate that the content of the ADC with D2+D6 is generally up to 81.31%. Those results indicate the method of the present application could modify the antibody with site-specific and prepare the different kinds of ADCs with improving the homogeneity.

TABLE 10

| | | The type | | | |
|---|---|---|---|---|---|
| | | of the | D0 | D1 | D2 |
| Example | Figure | ADCs | (wt %) | (wt %) | (wt %) |
| 115 | 57 | D1 | 11.0 | 83.61 | 5.39 |

TABLE 11

| | | The type | | | | | |
|---|---|---|---|---|---|---|---|
| | | of the | D0 | D2 | D0 + D4 | D0 + D6 | D8 |
| Example | Figure | ADCs | (%) | (%) | (%) | (%) | (%) |
| 116 | 58 | D0 + D6 | 0 | 0 | 4.17 | 84.68 | 11.16 |

TABLE 12

| | | The type | | | |
|---|---|---|---|---|---|
| | | of the | | | |
| Example | Figure | ADCs | D2 + D4/D4 + D2 (%) | D2 + D6 (%) | D4 + D4/D0 + D8 (%) |
| 117 | 59 | D2 + D6 | 7.49 | 81.31 | 11.2 |

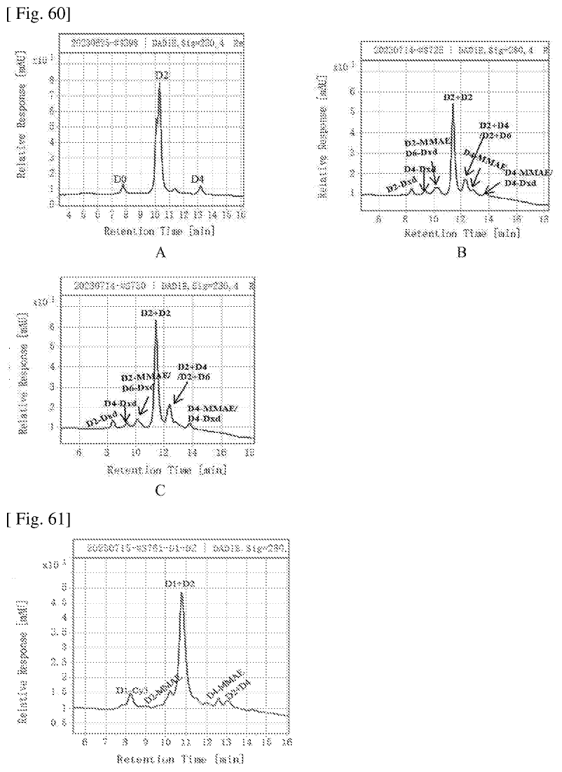

In step (3) of examples 118-119, one of the interchain disulfide bonds in the ADC with D2 was reduced. As shown in table 13, and FIG. 60, the results demonstrate that the content of the ADC with D2+D2 is generally up to 68% or 70%, which indicated the process of method was benefit for site-specific modifying the antibody with D2+D2 and improving the homogeneity.

TABLE 13

| No. | Figure | Reductant | D0(%) | D2 (%) | D4 (%) | / | / | / | / |
|---|---|---|---|---|---|---|---|---|---|
| E118- | 60A | TCEP-NO | 4.99 | 90.38 | 4.63 | / | / | / | / |
| step (2) | | | | | | | | | |

| | | | | | | D2 | | | |
|---|---|---|---|---|---|---|---|---|---|
| | | | D2 | D4 | D2 | (MMAE) + | D2 + D4/ | D4 | |
| | | | (DXd) | (DXd) | (MMAE)/ | D2 (DXd) | D2 + D6 | (MMAE) | D4 + D4 |
| No. | Figure | Reductant | (%) | (%) | D6(DXd)% | (%) | (%) | (%) | (%) |
| E118- | 60B | TCEP-3 | 4.32 | 2.83 | 9.59 | 68.47 | 10.80 | 1.73 | 2.26 |
| step (5) | | | | | | | | | |
| E119- | 60C | TCEP-6 | 4.95 | 2.06 | 6.77 | 70.55 | 12.50 | / | 3.18 |
| step (5) | | | | | | | | | |

As shown in table 14, and FIG. 61, the results demonstrate that the content of the ADC with D1+D2 is generally up to 80% or 83%, which indicated the process of method was benefit for site-specific modifying the antibody with D1+D2 and improving the homogeneity.

TABLE 14

| No. | Figure | D1(%) | D2 (%) | D1 + D2 (%) | D4 (%) | D2 + D4 (%) |
|---|---|---|---|---|---|---|
| E120 | 61 | 6.87 | 3.04 | 83.21 | 3.06 | 3.82 |

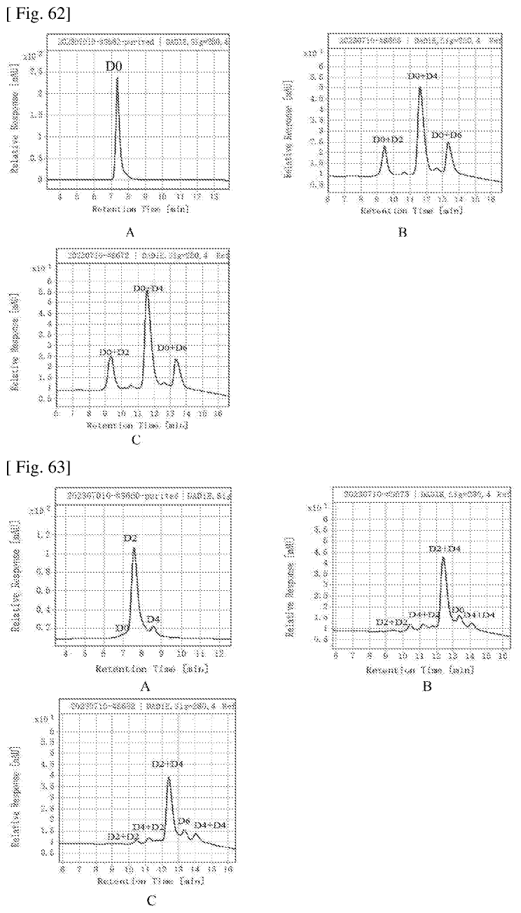

In step (3) of examples 121-122, two of the interchain disulfide bonds in the ADC with D2 was reduced. As shown in table 15, and FIG. 62, the results demonstrate that the content of the ADC with D0+D4 is generally up to 55% or 61%, which indicated the process of method was benefit for site-specific modifying the antibody with D0+D4 and improving the homogeneity.

TABLE 15

| No. | Figure | Reductant | D0(%) | / | / |
|---|---|---|---|---|---|
| E121-step (2) | 62A | TCEP-NO | 100 | / | / |

| | | | D0 + | D0 + | D0 + |
|---|---|---|---|---|---|
| No. | Figure | Reductant | D2 (%) | D4 (%) | D6 (%) |
| E121-step (5) | 62B | TCEP-3 | 18.96 | 61.27 | 19.77 |
| E122-step (5) | 62C | TCEP-6 | 21.23 | 61.85 | 16.92 |

As shown in table 16, and FIG. 63, the results demonstrate that the content of the ADC with D2+D4 is generally up to 70%, 75%, even to 78% or 80%, which indicated the process of method was benefit for site-specific modifying the antibody with D2+D4 and improving the homogeneity.

TABLE 16

| No. | Figure | Reductant | D0(%) | D2 (%) | / | D4 (%) | / |
|---|---|---|---|---|---|---|---|
| E123- | 63A | TCEP-NO | 3.99 | 87.26 | / | 8.75 | / |
| step (2) | | | | | | | |

| | | | D2 + D2 | D4 + D2 | D2 + D4 | D6 | D4 + D4 |
|---|---|---|---|---|---|---|---|
| No. | Figure | Reductant | (%) | (%) | (%) | (%) | (%) |
| E123- | 63B | TCEP-3 | 5.34 | 4.01 | 79.67 | 6.78 | 4.21 |
| step (5) | | | | | | | |
| E124- | 63C | TCEP-6 | 3.03 | 3.53 | 79.37 | 6.93 | 7.14 |
| step (5) | | | | | | | |

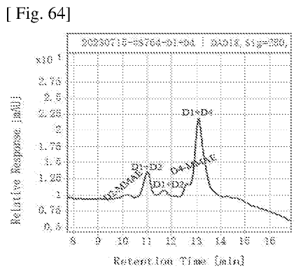

As shown in table 17, and FIG. 64, the results demonstrate that the content of the ADC with D1+D4 is generally up to 60%, 65%, even to 70%, which indicated the process of method was benefit for site-specific modifying the antibody with D1+D4 and improving the homogeneity.

TABLE 17

| | | | D2 | D1 + D2 | D4 | D1 + D4 |
|---|---|---|---|---|---|---|
| No. | Figure | Reductant | (%) | (%) | (%) | (%) |
| E125 | 64 | TCEP-3 | 3.09 | 23.96 | 2.45 | 70.49 |

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications, variations, improvements, and substantial equivalents.

1.-79. (canceled)

80. A compound having the following formula (I): 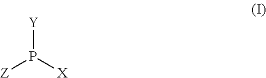
or a salt, solvate, stereoisomer thereof, which characterized in that,
X, Y and Z independently covalently connect the phosphorus atom through P—C bond, which is P—C(sp³) or P—C(sp²);
X is of formula (II): 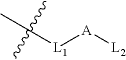
L₁ is selected from the group consisting of —CH(R₁)—, —C(CH₃)(R₁)—, —CH(R₁)CH(R₂)—, —CH(R₁)CH(R₂)CH(R₃)—, aryl group which is unsubstituted or independently substituted with group containing at least a coordinating atom selected from N, O and S, and heteroaryl group which is unsubstituted or independently substituted with group containing at least a coordinating atom selected from O and S;
R₁, R₂ and R₃ independently are H, C₁-C₅ alkyl group, C₁-C₅ hydroxyalkyl group, C₁-C₅ carboxy alkyl group, C₁-C₅ hydroxylamine alkyl group, C₁-C₅ N-hydroxy amide alkyl group, aryl group or heteroaryl group; or
R₂ or R₃ forms a 5-6 membered unsubstituted or substituted ring with L₂;
A is not present or present, when A is present and A is —C(O)—, or —C(O)J-;
J is organic group comprising amino or imino group and carbonyl group at the same time, of which the amino or imino group forms amide group with —C(O), the carboxyl group unlinked or covalently linked to L₂;
L₂ is not present or present, when L₂ is present and L₂ works as transition metal chelator motif and is —N(R₄)(R₅) or hydroxy;
R₄ and R₅ independently are hydrogen, C₀-C₅ hydroxyalkyl group, C₁-C₅ alkyl group, C₁-C₅ alkoxy group, —(CH₂)n¹(OCH₂CH₂O)n²CH(R₆)CO(R₇), unsubstituted or substituted 5-6 membered saturated heterocyclic group, unsubstituted or substituted arylalkyl group, unsubstituted or substituted aryl alkoxy group, unsubstituted or substituted aryl group, unsubstituted or substituted heteroaryl group, unsubstituted or substituted heteroaryl alkyl group, or R₄ and R₅ form a 5-6 membered unsubstituted or substituted ring, R₄ or R₅ forms a 5-6 membered unsubstituted or substituted ring with R₂ or R₃;
R₆ is hydrogen, amino, C₁-C₈alkyl, C₁-C₅ hydroxyalkyl group, C₁-C₅ carboxy alkyl group, aryl group, unsubstituted or substituted arylalkyl group, C₁-C₅ N-hydroxy amide alkyl group, heteroaryl group or heteroaryl alkyl group;
R₇ is hydroxy, C₁-C₈alkoxy group, —NH(CH₂CONH)n³OH;
n¹, n² and n³ independently are the number 0, 1, 2, 3, 4;
R₄ and R₅ are not hydroxy at the same time;
Y is same as X,
Z is same as X, or
Y and Z independently are 5-6 membered unsubstituted or substituted saturated heterocyclic group, C₁-C₅ alkyl group, C₁-C₅ hydroxyalkyl group, aryl group, C₁-C₅ carboxy alkyl group, 5-6 membered unsubstituted or substituted cycloalkyl group, or 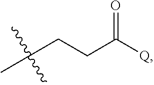
—C(O)Q is ester group, imide group or amide group,
X, Y and Z are not —CH₂CH₂C(O)OH at the same time.

81. The compound of claim 80, which characterized in that,
L₁ is —CH(R₁)CH(R₂)—,
R₁ and R₂ independently are H, methyl group, isopropyl group, hydroxymethyl group, hydroxyethyl group, carboxy methyl group, carboxy ethyl group, N-hydroxy ethyl amide group, phenyl group, 2-pyridyl group, 4-pyridyl group or 4-imidazole group, or
R₂ forms a 5-6 membered unsubstituted or substituted ring with L₂.

82. The compound of claim 81, which characterized in that,
L₁ is —CH(R₁)CH(R₂)—;
R₁ is H, and
R₂ forms a 5-6 membered unsubstituted or substituted ring with R₄ of L₂;
L₂ is —N(R₄)(R₅), R₅ is hydroxy.

83. The compound of claim 80, which characterized in that,
L₁ is unsubstituted phenyl group or phenyl group substituted with hydroxy or carboxy group, in ortho or meta position, and the phenyl group connected to A in ortho, meta or para position,
A is —C(O)—;
L₂ is —N(R₄)(R₅) or hydroxy;
R₄ is hydrogen, R₅ is hydroxy.

84. The compound of claim 80, which characterized in that,
L₁ is phenyl group which is unsubstituted or substituted with hydroxy, halogen, carboxyl, sulfonyl, amino, methoxy or ethoxy in ortho, meta or para position, or L₁ is unsubstituted or substituted 4-pyridyl group, or unsubstituted or substituted 4-quinolyl group;
A and L₂ are not present.

85. The compound of claim 84, which characterized in that,
L₁ is

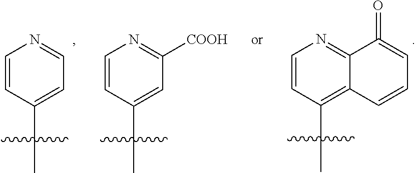

86. The compound of claim 81, which characterized in that,
L₁ is —CH(R₁)CH(R₂)—,
R₁ is methyl group, isopropyl group, carboxy ethyl group or N-hydroxy ethyl amide group,
R₂ is H,
A is —C(O)—;
L₂ is —N(R₄)(R₅);
R₄ is hydrogen, and R₅ is hydroxy.

87. The compound of claim 81, which characterized in that,
L₁ is —CH(R₁)CH(R₂)—,
R₁ is H,
R₂ is methyl group, hydroxymethyl group, hydroxyethyl group, carboxy ethyl group, phenyl group, N-hydroxy ethyl amide group, 2-pyridyl group, 4-pyridyl group or 4-imidazole group,
A is —C(O)—;
L₂ is —N(R₄)(R₅);
R₄ is hydrogen, unsubstituted or substituted 5-6 membered saturated heterocyclic group,
R₅ is hydroxy,
or R₄ and R₅ form a 5-6 membered unsubstituted or substituted ring.

88. The compound of claim 87, which characterized in that,
R₄ is hydrogen, or.

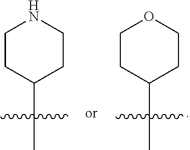 [SMILES: *C1CCNCC1 ; *C1CCOCC1]

89. The compound of claim 87, which characterized in that,
L₂ is N Or N

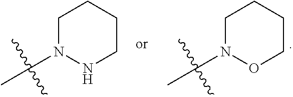 [SMILES: *N1CCCCN1 ; *N1CCCCO1]

90. The compound of claim 80, which characterized in that,
L₁ is —CH(R₁)CH(R₂)—;
R₁ and R₂ are H;
L₂ is —N(R₄)(R₅);
R₄ is hydrogen, C₁-C₅ alkyl group, —(CH₂)n¹(OCH₂CH₂O)n²CH(R₆)CO(R₇), unsubstituted or substituted 5-6 membered saturated heterocyclic group, unsubstituted or substituted arylalkyl group, unsubstituted or substituted aryl group, unsubstituted or substituted heteroaryl alkyl group or R₄ and R₅ form a 5-6 membered unsubstituted or substituted ring;
R₅ is hydroxy,
R₆ is hydrogen, amino, C₁-C₅ alkyl, C₁-C₅ hydroxyalkyl group, C₁-C₅ carboxy alkyl group, aryl group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4.

91. The compound of claim 90, which characterized in that,
R₄ is hydrogen, methyl group, —(CH₂)n¹(OCH₂CH₂O)n²CH(R₆)CO(R₇), 5-6 membered saturated heterocyclic group which comprises a heteroatom N or O, benzyl group, benzyl group which is substituted with hydroxy on the phenyl ring, phenyl which is unsubstituted or substituted with hydroxy, halogen or carboxyl group, heteroaryl alkyl group which comprises a heteroatom N, or R₄ and R₅ form a 5-6 membered ring;
R₅ is hydroxy,
R₆ is hydrogen, C₁-C₅ alkyl, C₁-C₅ hydroxyalkyl group, or heteroaryl alkyl group;
R₇ is hydroxy, C₁-C₈alkoxy group, —NH(CH₂CONH)n³OH;
n¹, n² and n³ independently are the number 0, 1, 2, 3, 4.

92. The compound of claim 90, which characterized in that,
R₄ is, 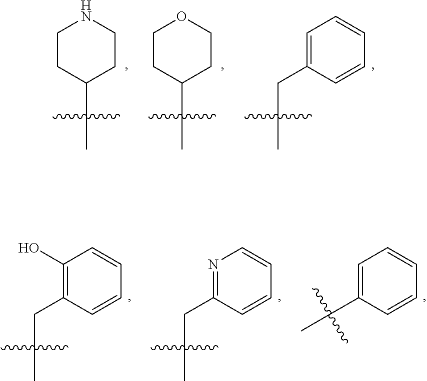
hydrogen or —(CH₂)n¹(OCH₂CH₂O)n²CH(R₆)CO(R₇),
R₅ is hydroxy,
R₆ is hydrogen, methyl group, hydroxymethyl group or 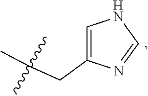 [SMILES: *Cc1c[nH]cn1]
R₇ is hydroxy or —NH(CH₂CONH)n³OH;
n¹, n² and n³ independently are the number 0.

93. The compound of claim 80, which characterized in that,
L₁ is —CH(R₁)CH(R₂)—;
R₁ and R₂ independently are H;
L₂ is —N(R₄)(R₅);
R₄ and R₅ are independently —(CH₂)n¹(OCH₂CH₂O)n²CH(R₆)CO(R₇) or unsubstituted or substituted heteroaryl alkyl group,
R₆ is hydrogen, amino, C₁-C₅ alkyl, C₁-C₅ hydroxyalkyl group, C₁-C₅ carboxy alkyl group, aryl group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4.

94. The compound of claim 93, which characterized in that,
R₄ and R₅ are independently —(CH₂)n¹(OCH₂CH₂O)n²CH(R₆)CO(R₇) or 6 membered heteroaryl alkyl group;
R₆ is hydrogen;
R₇ is hydroxy, C₁-C₅ alkoxy group, —NH(CH₂CONH)n³OH;
n¹, n² and n³ independently are the number 0, 1, 2, 3, 4.

95. The compound of claim 93, which characterized in that,
R₄ and R₅ are independently —(CH₂)n¹(OCH₂CH₂O)n²CH(R₆)CO(R₇) or 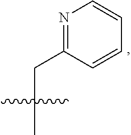 [SMILES: *Cc1ccccn1]
R₆ is hydrogen,
R₇ is hydroxy or —NH(CH₂CONH)n³OH;
n¹, n² and n³ independently are the number 0.

96. The compound of claim 80, which characterized in that,
L₁ is —CH(R₁)CH(R₂)—;
R₁ and R₂ independently are H;
L₂ is —N(R₄)(R₅);
R₄ is hydrogen, C₀-C₅ hydroxyalkyl group, C₁-C₅ alkyl group, unsubstituted or substituted C₁-C₅ alkoxy group, —(CH₂)n¹(OCH₂CH₂O)n²CH(R₆)CO(R₇), unsubstituted or substituted arylalkyl group, unsubstituted or substituted aryl alkoxy group, unsubstituted or substituted aryl group, unsubstituted or substituted heteroaryl group, unsubstituted or substituted heteroaryl alkyl group;
R₅ is hydrogen,
R₆ is hydrogen, amino, C₁-C₅ alkyl, C₁-C₅ hydroxyalkyl group, C₁-C₅ carboxy alkyl group, aryl group, unsubstituted or substituted arylalkyl group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4.

97. The compound of claim 96, which characterized in that,
R₄ is hydrogen, C₀-C₃ hydroxyalkyl group, C₁-C₃ alkoxy group, —(CH₂)n¹(OCH₂CH₂O)n²CH(R₆)CO(R₇), phenyl group which is substituted with carboxy, hydroxy, amino, halogen, pyridyl group, amino which is substituted with 2-methylpyridine, benzyl group which is substituted with carboxy, hydroxy, amino or halogen, aryl alkoxy group, pyridyl group which is substituted with carboxy, bipyridyl group, 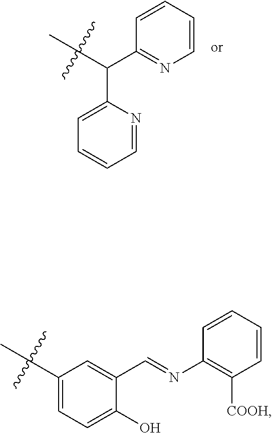 [SMILES: *C(c1ccccn1)c1ccccn1 ; *c1ccc(O)c(/C=N/c2ccccc2C(=O)O)c1]
or OH,
R₅ is hydrogen,
R₆ is hydrogen, amino, C₁-C₃ alkyl, C₁-C₃ hydroxyalkyl group, C₁-C₃ carboxy alkyl group, aryl group, arylalkyl group which is unsubstituted or substituted with hydroxy group, halogen, cyano group or nitro group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4.

98. The compound of claim 97, which characterized in that,
R₄ is hydrogen, hydroxy, ethyl hydroxyl group, methoxy group, 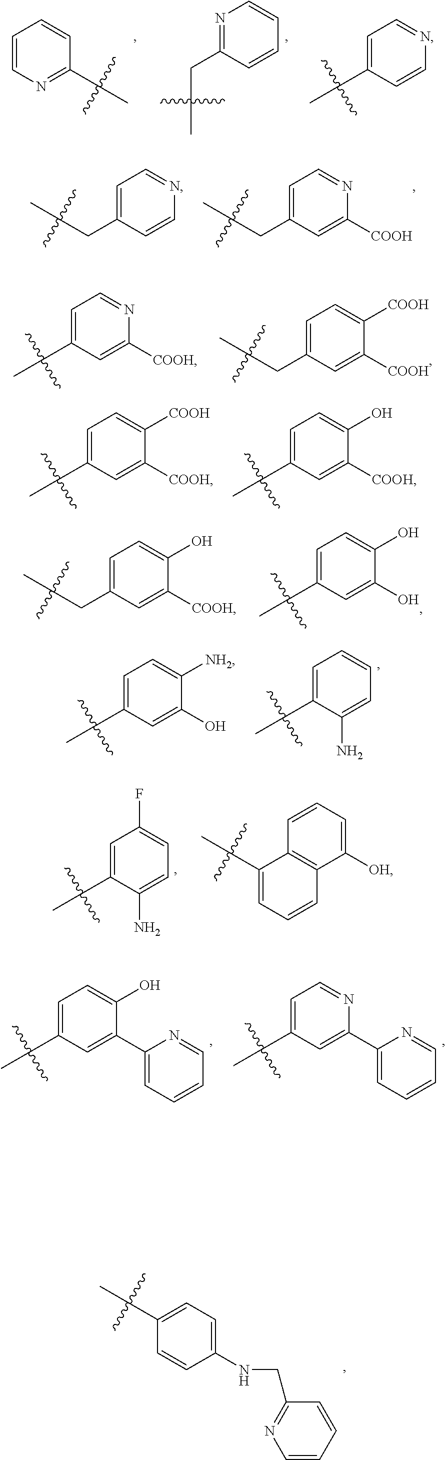 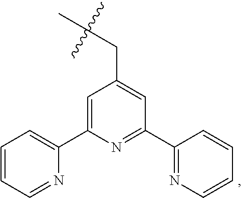 [SMILES: *Cc1cc(-c2ccccn2)nc(-c2ccccn2)c1] 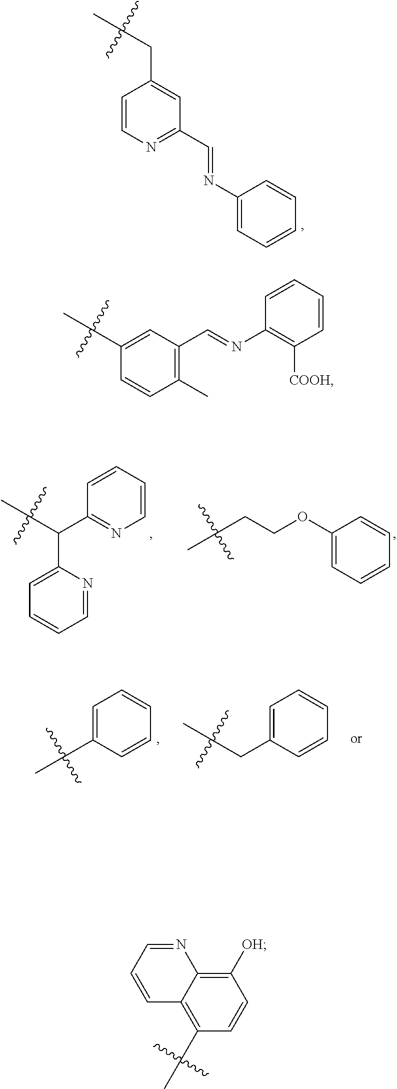
R₅ is hydrogen.

99. The compound of claim 96, which characterized in that,
R₄ is —(CH₂)n¹(OCH₂CH₂O)n²CH(R₆)CO(R₇),
R₅ is hydrogen,
R₆ is hydrogen, amino, methyl, hydroxymethyl group, carboxy ethyl group, benzyl group, benzyl group substituted with —OH, F, —CN or —NO₂, N-hydroxy ethyl amide group, 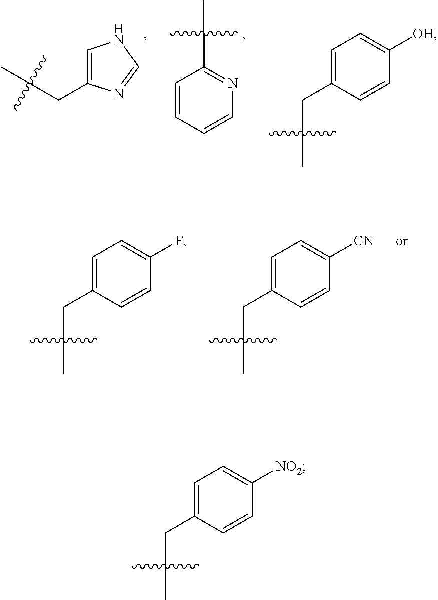
R₇ is hydroxy, —NH(CH₂CONH)n³OH;
n¹ and n³ independently are the number 0, 1, 2, 3, 4,
n² is the number 0 or 1.

100. The compound of claim 80, which characterized in that,
J is peptide residue comprising mono amino acid residue, dipeptide, tripeptide, tetrapeptide, pentapeptide, aminopropionic acid, aminobutyric acid, amino valeric acid, aminoacid, aminoheptanoic acid, aminooctanoic acid, or —NH(OCH₂CH₂O)n⁴CH₂COOH, n⁴ is the number of 2-10,
the amino acid is selected from the group consisting of glycine (Gly), alanine (Ala), serine (Ser), arginine (Arg), asparagine (Asn), asparticacid (Asp), cysteine (Cys), glutamine (Gln), glutamicacid (Glu), histidine (His), isoleucine (Ile), leucine (Leu), lysine (Lys), methionine (Met), phenylalanine (Phe), proline (Pro), threonine (Thr), tryptophan (Trp), tyrosine (Tyr) and valine (Val).

101. The compound of claim 100, which characterized in that,
J is the residue of histidine, serine, alanine, glycine, phenylalanine, asparagine, tyrosine or asparagine.

102. The compound of claim 100, which characterized in that,
A is —C(O)J-,
J is the residue of histidine, serine, alanine, glycine, phenylalanine, asparagine, tyrosine or asparagine,
L₂ is —N(R₄)(R₅), R₄ is hydrogen, R₅ is hydroxy.

103. The compound of claim 80, which characterized in that,
Y and Z independently are 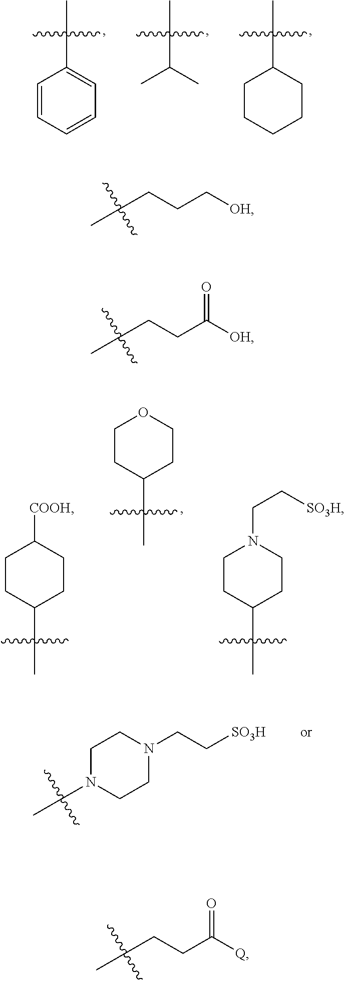
Q is —NHOH, —NHCH₂CH₂SO₃H, —N(CH₂CH₂OH)₂, —NHCH₂COOH, —NHCH(CH₃)COOH, —NH(CH₂CH₂O)₃CH₃.

104. The compound of claim 80, which characterized in that, the compound is selected from the group consisting of

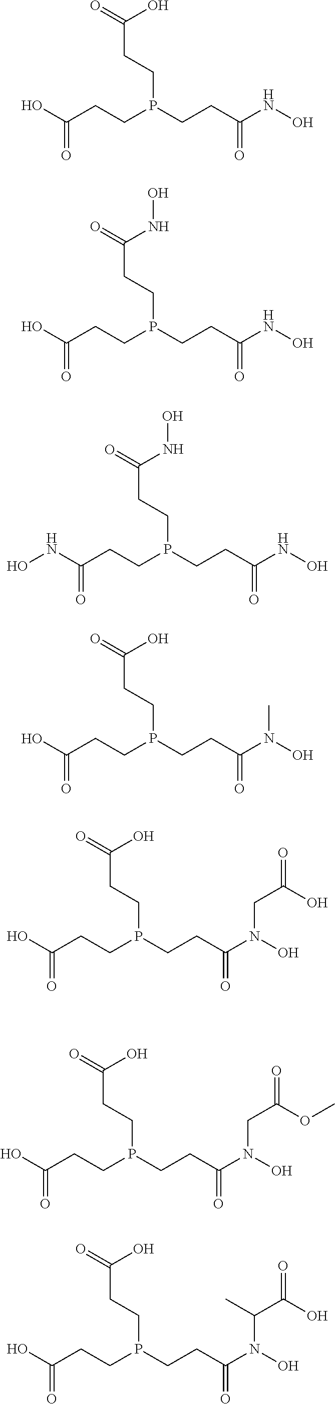

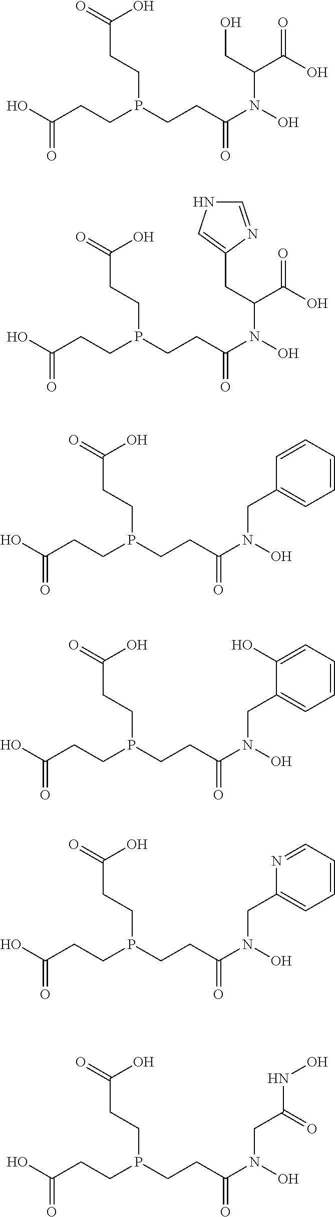 [SMILES: O=C(O)CCP(CCC(=O)O)CCC(=O)N(O)C(CO)C(=O)O ; O=C(O)CCP(CCC(=O)O)CCC(=O)N(O)C(Cc1c[nH]cn1)C(=O)O ; O=C(O)CCP(CCC(=O)O)CCC(=O)N(O)Cc1ccccc1 ; O=C(O)CCP(CCC(=O)O)CCC(=O)N(O)Cc1ccccc1O ; O=C(O)CCP(CCC(=O)O)CCC(=O)N(O)Cc1ccccn1 ; O=C(O)CCP(CCC(=O)O)CCC(=O)N(O)CC(=O)NO]

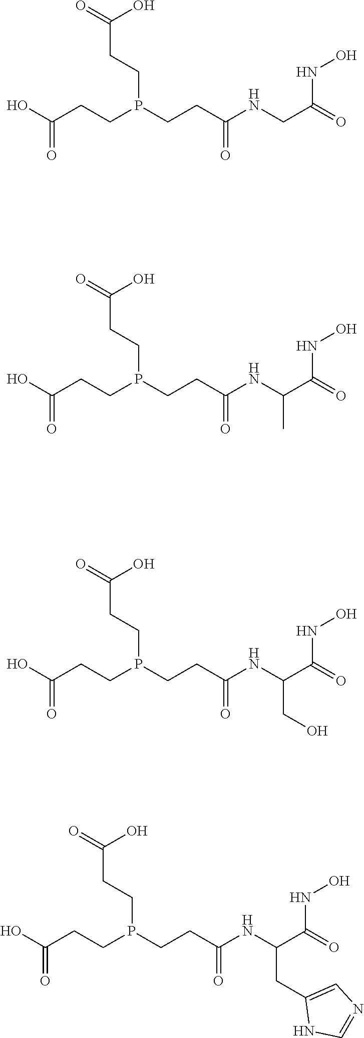 [SMILES: O=C(O)CCP(CCC(=O)O)CCC(=O)NCC(=O)NO ; CC(NC(=O)CCP(CCC(=O)O)CCC(=O)O)C(=O)NO ; O=C(O)CCP(CCC(=O)O)CCC(=O)NC(CO)C(=O)NO ; O=C(O)CCP(CCC(=O)O)CCC(=O)NC(Cc1cnc[nH]1)C(=O)NO]

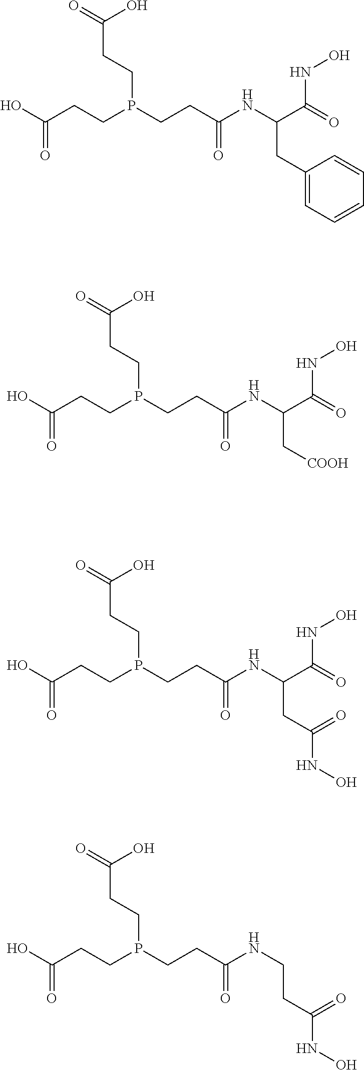 [SMILES: O=C(O)CCP(CCC(=O)O)CCC(=O)NC(Cc1ccccc1)C(=O)NO ; O=C(O)CCP(CCC(=O)O)CCC(=O)NC(CC(=O)O)C(=O)NO ; O=C(O)CCP(CCC(=O)O)CCC(=O)NC(CC(=O)NO)C(=O)NO ; O=C(O)CCP(CCC(=O)O)CCC(=O)NCCC(=O)NO]

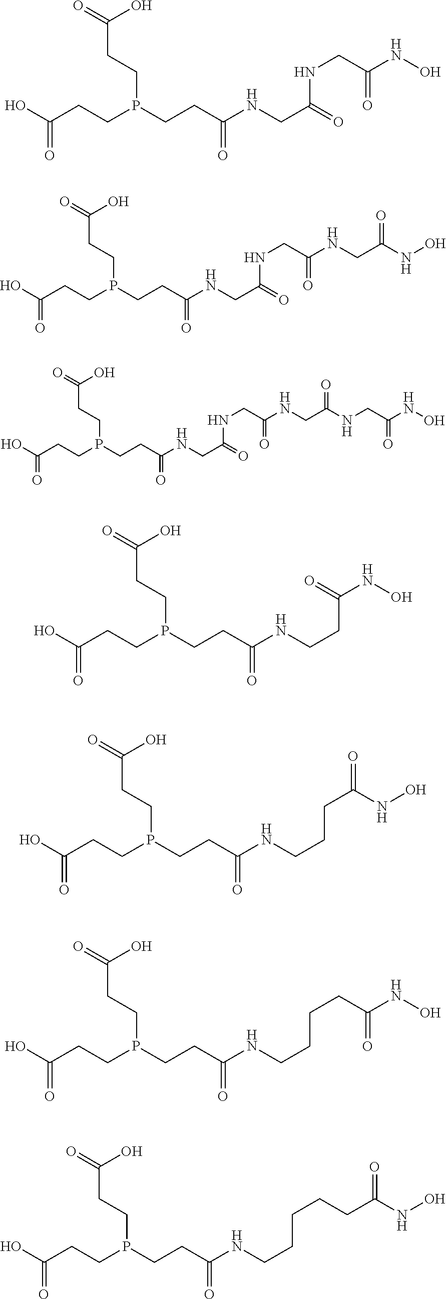

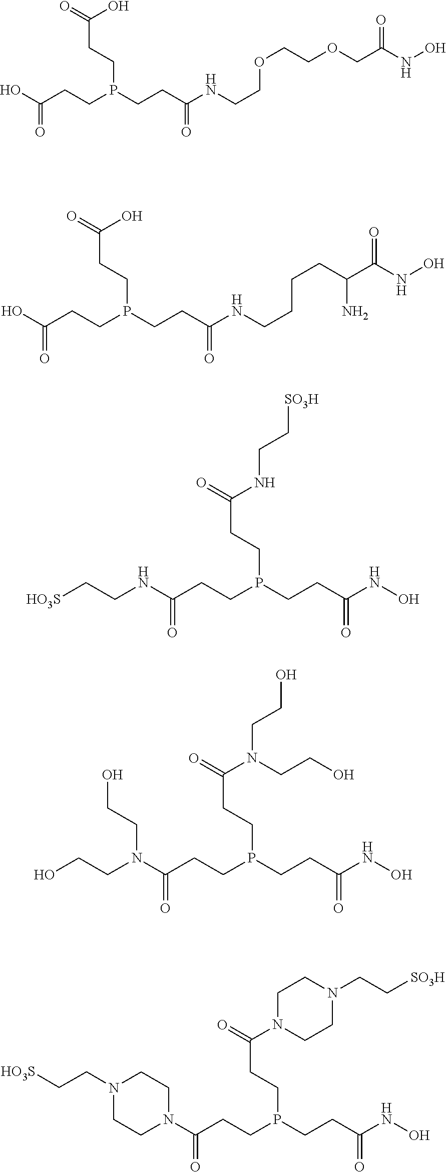 [SMILES: O=C(O)CCP(CCC(=O)O)CCC(=O)NCCOCCOCC(=O)NO ; NC(CCCCNC(=O)CCP(CCC(=O)O)CCC(=O)O)C(=O)NO ; O=C(CCP(CCC(=O)NCCS(=O)(=O)O)CCC(=O)NCCS(=O)(=O)O)NO ; O=C(CCP(CCC(=O)N(CCO)CCO)CCC(=O)N(CCO)CCO)NO ; O=C(CCP(CCC(=O)N1CCN(CCS(=O)(=O)O)CC1)CCC(=O)N1CCN(CCS(=O)(=O)O)CC1)NO]

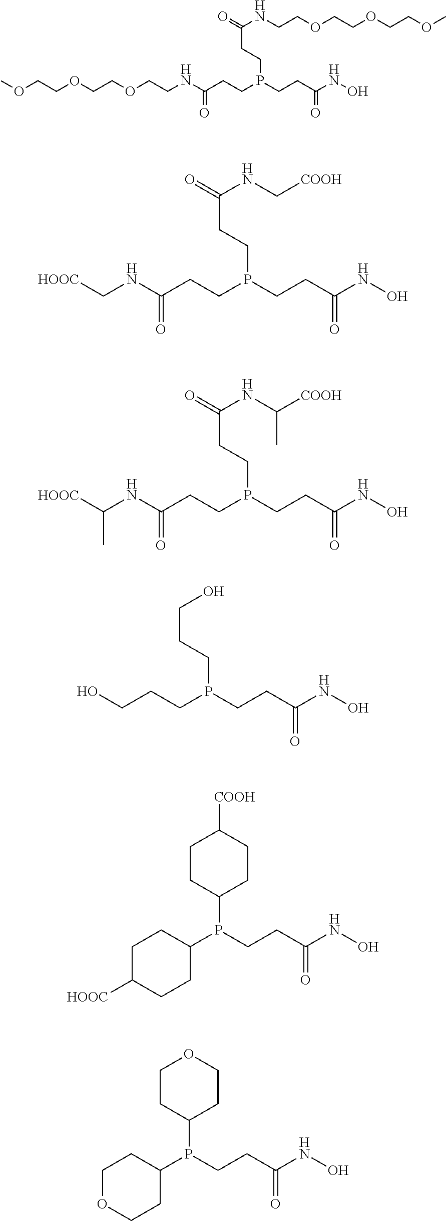

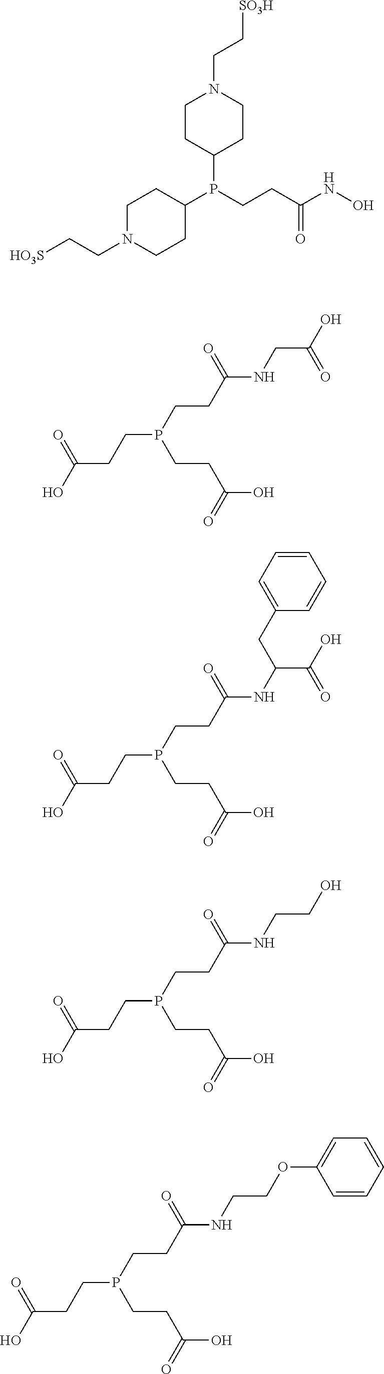 [SMILES: O=C(CCP(C1CCN(CCS(=O)(=O)O)CC1)C1CCN(CCS(=O)(=O)O)CC1)NO ; O=C(O)CCP(CCC(=O)O)CCC(=O)NCC(=O)O ; O=C(O)CCP(CCC(=O)O)CCC(=O)NC(Cc1ccccc1)C(=O)O ; O=C(O)CCP(CCC(=O)O)CCC(=O)NCCO ; O=C(O)CCP(CCC(=O)O)CCC(=O)NCCOc1ccccc1]

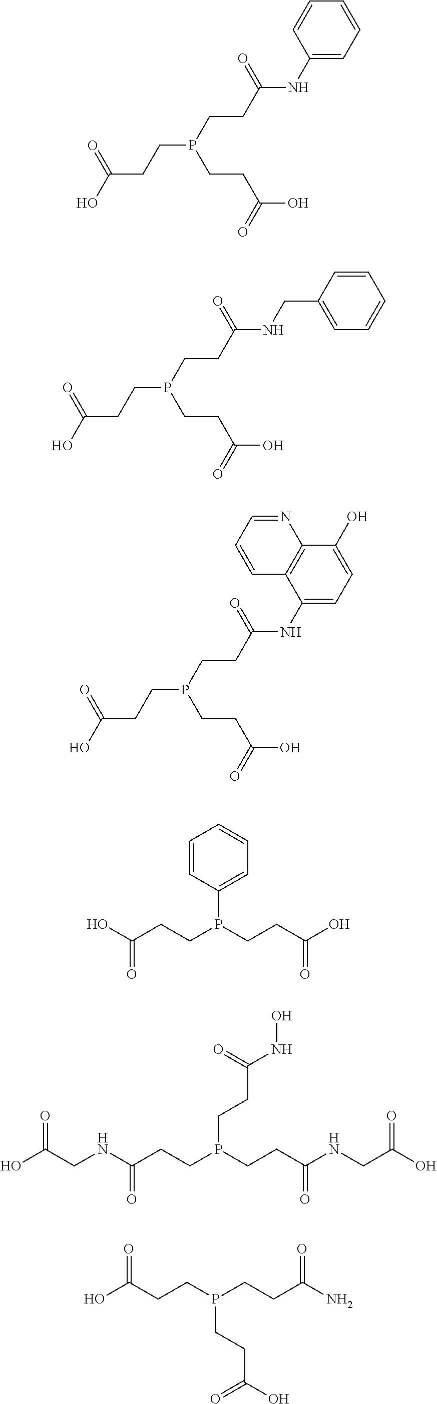 [SMILES: O=C(O)CCP(CCC(=O)O)CCC(=O)Nc1ccccc1 ; O=C(O)CCP(CCC(=O)O)CCC(=O)NCc1ccccc1 ; O=C(O)CCP(CCC(=O)O)CCC(=O)Nc1ccc(O)c2ncccc12 ; O=C(O)CCP(CCC(=O)O)c1ccccc1 ; O=C(O)CNC(=O)CCP(CCC(=O)NO)CCC(=O)NCC(=O)O ; NC(=O)CCP(CCC(=O)O)CCC(=O)O]

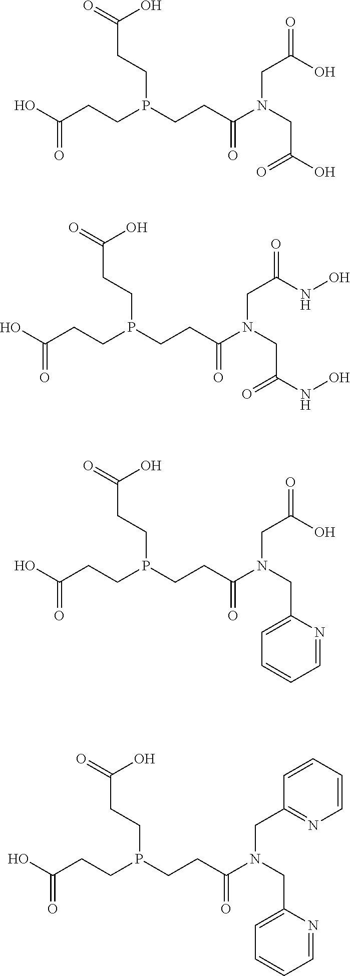 [SMILES: O=C(O)CCP(CCC(=O)O)CCC(=O)N(CC(=O)O)CC(=O)O ; O=C(O)CCP(CCC(=O)O)CCC(=O)N(CC(=O)NO)CC(=O)NO ; O=C(O)CCP(CCC(=O)O)CCC(=O)N(CC(=O)O)Cc1ccccn1 ; O=C(O)CCP(CCC(=O)O)CCC(=O)N(Cc1ccccn1)Cc1ccccn1]

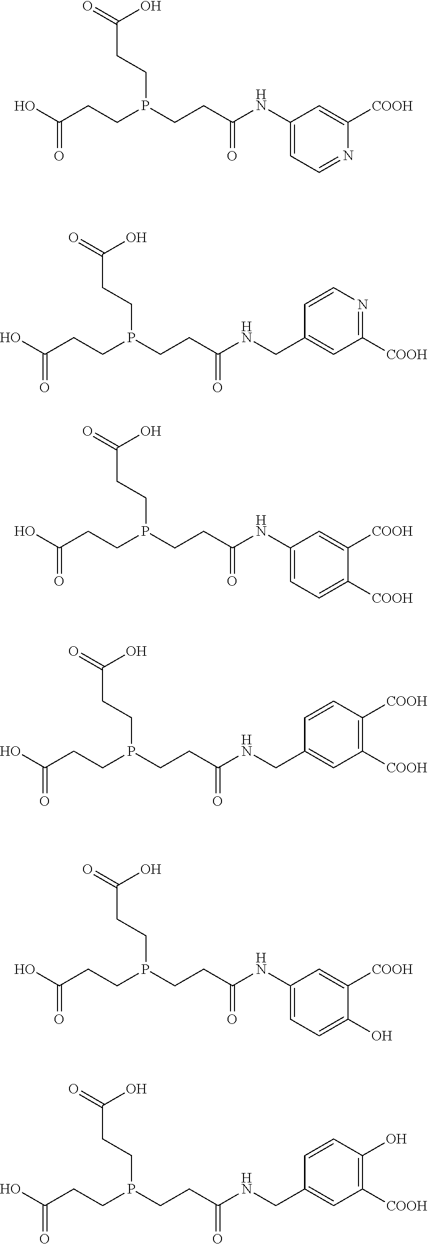 [SMILES: O=C(O)CCP(CCC(=O)O)CCC(=O)Nc1ccnc(C(=O)O)c1 ; O=C(O)CCP(CCC(=O)O)CCC(=O)NCc1ccnc(C(=O)O)c1 ; O=C(O)CCP(CCC(=O)O)CCC(=O)Nc1ccc(C(=O)O)c(C(=O)O)c1 ; O=C(O)CCP(CCC(=O)O)CCC(=O)NCc1ccc(C(=O)O)c(C(=O)O)c1 ; O=C(O)CCP(CCC(=O)O)CCC(=O)Nc1ccc(O)c(C(=O)O)c1 ; O=C(O)CCP(CCC(=O)O)CCC(=O)NCc1ccc(O)c(C(=O)O)c1]

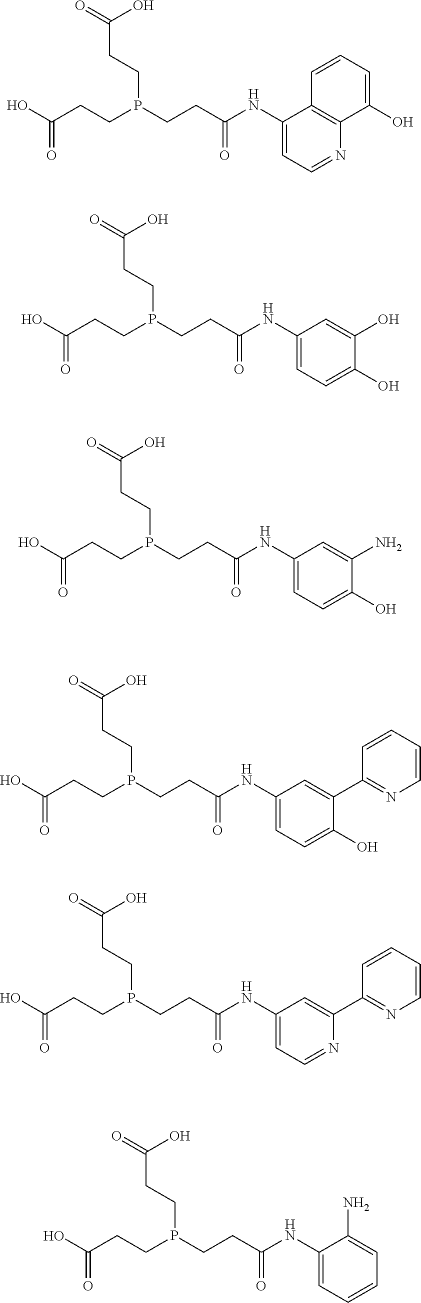 [SMILES: O=C(O)CCP(CCC(=O)O)CCC(=O)Nc1ccnc2c(O)cccc12 ; O=C(O)CCP(CCC(=O)O)CCC(=O)Nc1ccc(O)c(O)c1 ; Nc1cc(NC(=O)CCP(CCC(=O)O)CCC(=O)O)ccc1O ; O=C(O)CCP(CCC(=O)O)CCC(=O)Nc1ccc(O)c(-c2ccccn2)c1 ; O=C(O)CCP(CCC(=O)O)CCC(=O)Nc1ccnc(-c2ccccn2)c1 ; Nc1ccccc1NC(=O)CCP(CCC(=O)O)CCC(=O)O]

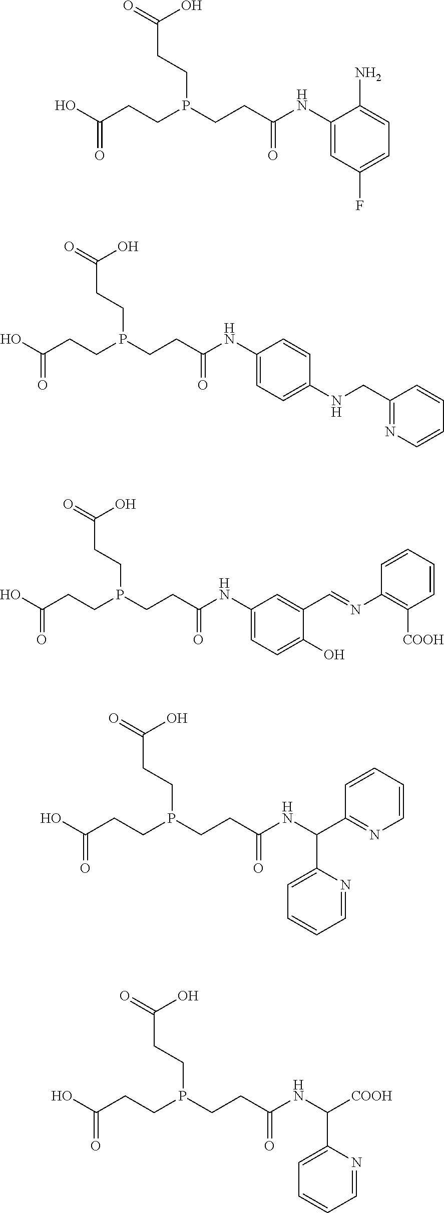 [SMILES: Nc1ccc(F)cc1NC(=O)CCP(CCC(=O)O)CCC(=O)O ; O=C(O)CCP(CCC(=O)O)CCC(=O)Nc1ccc(NCc2ccccn2)cc1 ; O=C(O)CCP(CCC(=O)O)CCC(=O)Nc1ccc(O)c(/C=N/c2ccccc2C(=O)O)c1 ; O=C(O)CCP(CCC(=O)O)CCC(=O)NC(c1ccccn1)c1ccccn1 ; O=C(O)CCP(CCC(=O)O)CCC(=O)NC(C(=O)O)c1ccccn1]

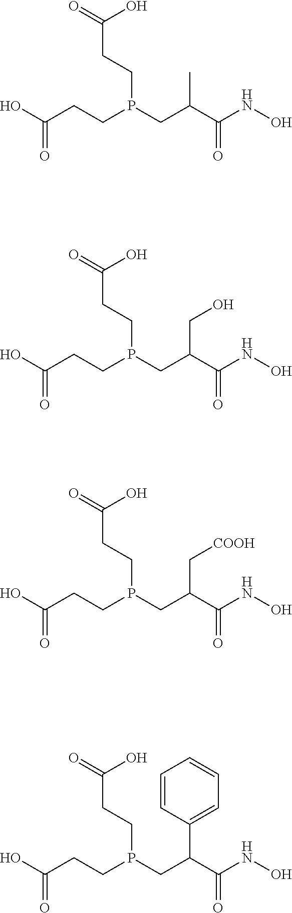 [SMILES: CC(CP(CCC(=O)O)CCC(=O)O)C(=O)NO ; O=C(O)CCP(CCC(=O)O)CC(CO)C(=O)NO ; O=C(O)CCP(CCC(=O)O)CC(CC(=O)O)C(=O)NO ; O=C(O)CCP(CCC(=O)O)CC(C(=O)NO)c1ccccc1]

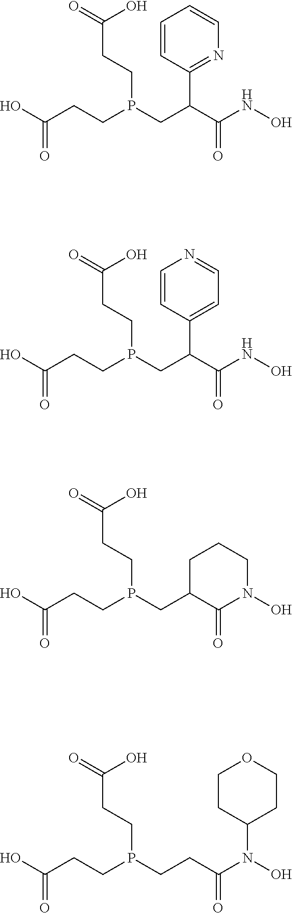 [SMILES: O=C(O)CCP(CCC(=O)O)CC(C(=O)NO)c1ccccn1 ; O=C(O)CCP(CCC(=O)O)CC(C(=O)NO)c1ccncc1 ; O=C(O)CCP(CCC(=O)O)CC1CCCN(O)C1=O ; O=C(O)CCP(CCC(=O)O)CCC(=O)N(O)C1CCOCC1]

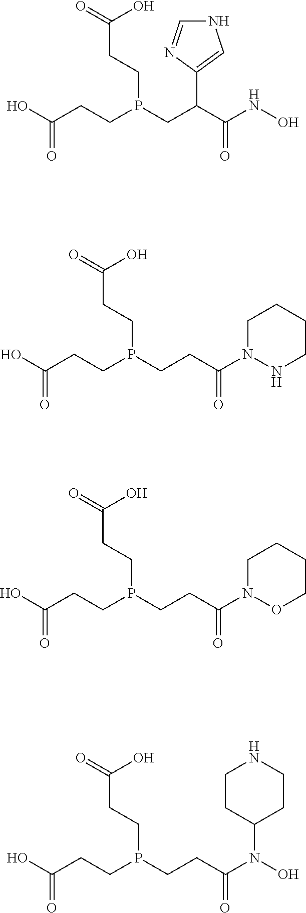 [SMILES: O=C(O)CCP(CCC(=O)O)CC(C(=O)NO)c1c[nH]cn1 ; O=C(O)CCP(CCC(=O)O)CCC(=O)N1CCCCN1 ; O=C(O)CCP(CCC(=O)O)CCC(=O)N1CCCCO1 ; O=C(O)CCP(CCC(=O)O)CCC(=O)N(O)C1CCNCC1]

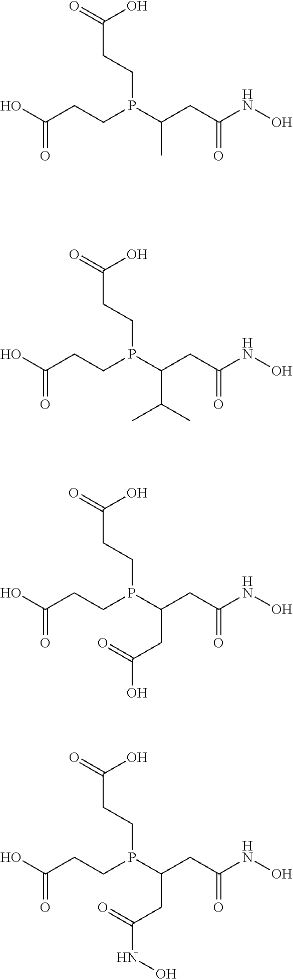 [SMILES: CC(CC(=O)NO)P(CCC(=O)O)CCC(=O)O ; CC(C)C(CC(=O)NO)P(CCC(=O)O)CCC(=O)O ; O=C(O)CCP(CCC(=O)O)C(CC(=O)O)CC(=O)NO ; O=C(O)CCP(CCC(=O)O)C(CC(=O)NO)CC(=O)NO]

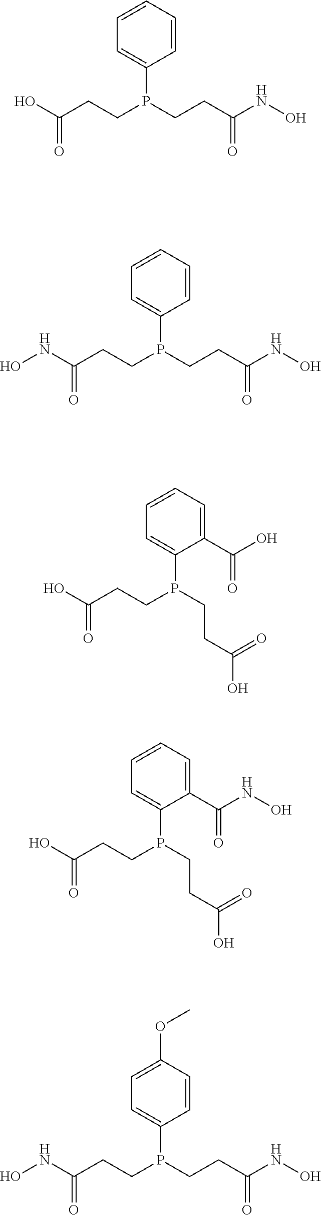 [SMILES: O=C(O)CCP(CCC(=O)NO)c1ccccc1 ; O=C(CCP(CCC(=O)NO)c1ccccc1)NO ; O=C(O)CCP(CCC(=O)O)c1ccccc1C(=O)O ; O=C(O)CCP(CCC(=O)O)c1ccccc1C(=O)NO ; COc1ccc(P(CCC(=O)NO)CCC(=O)NO)cc1]

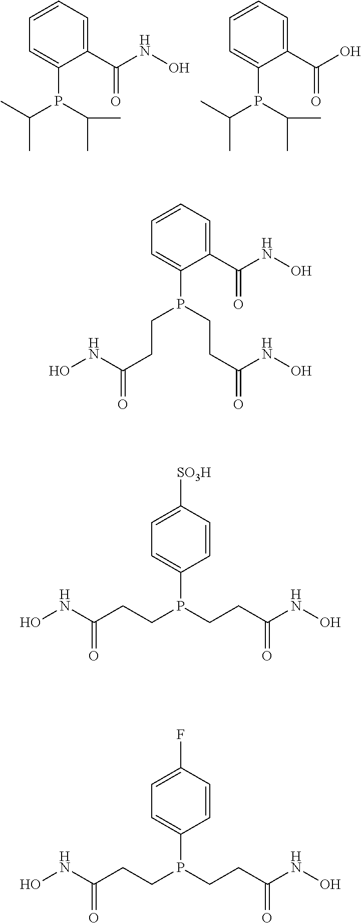

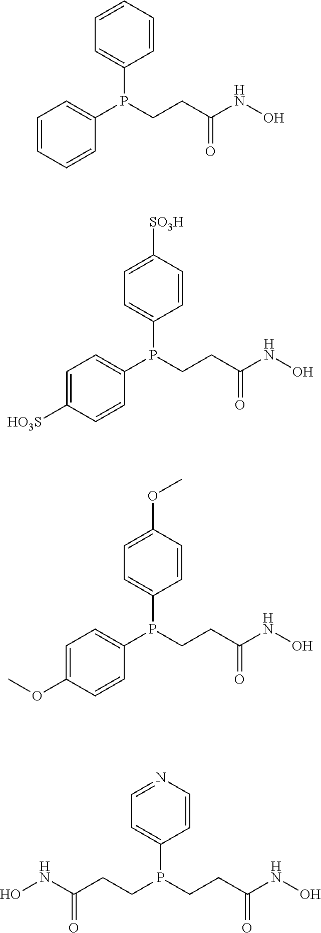 [SMILES: O=C(CCP(c1ccccc1)c1ccccc1)NO ; O=C(CCP(c1ccc(S(=O)(=O)O)cc1)c1ccc(S(=O)(=O)O)cc1)NO ; COc1ccc(P(CCC(=O)NO)c2ccc(OC)cc2)cc1 ; O=C(CCP(CCC(=O)NO)c1ccncc1)NO]

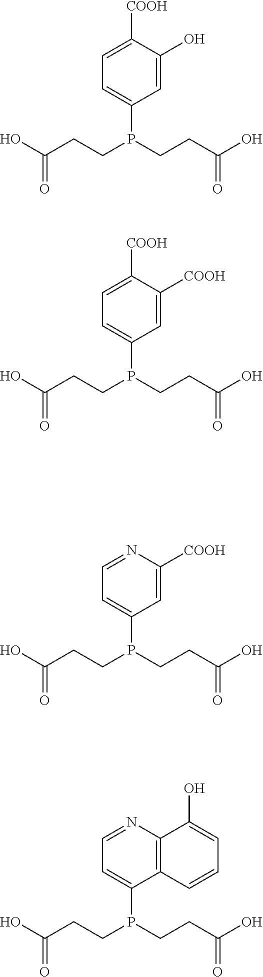 [SMILES: O=C(O)CCP(CCC(=O)O)c1ccc(C(=O)O)c(O)c1 ; O=C(O)CCP(CCC(=O)O)c1ccc(C(=O)O)c(C(=O)O)c1 ; O=C(O)CCP(CCC(=O)O)c1ccnc(C(=O)O)c1 ; O=C(O)CCP(CCC(=O)O)c1ccnc2c(O)cccc12]

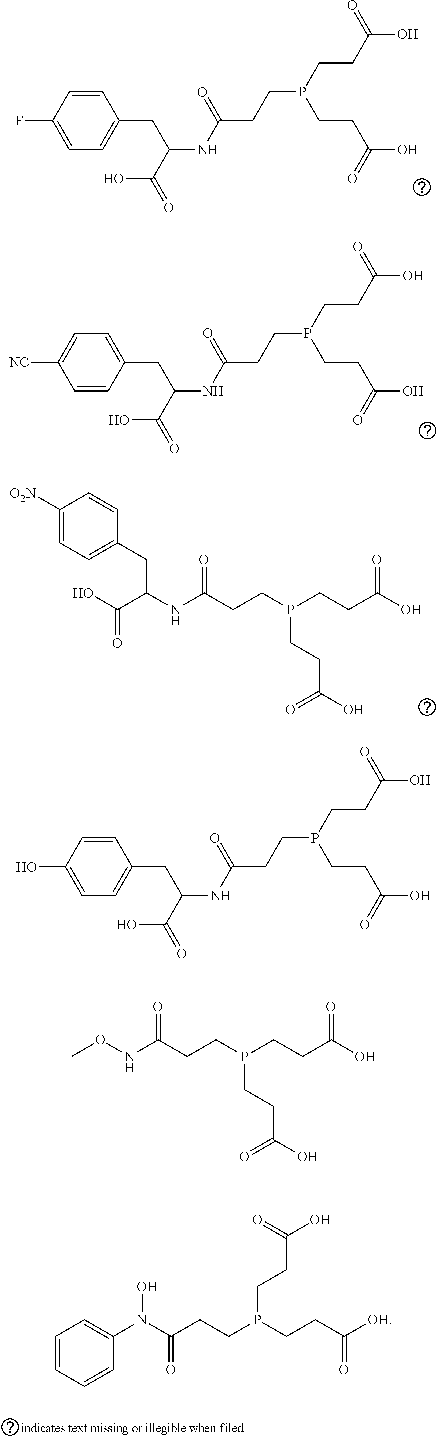

105. A composition comprising a compound according to claim 80 and transition metal ions.

106. The composition according to claim 105, which characterized in that, the transition metal ion is Zn²⁺, Cd²⁺, Hg²⁺, Ni²⁺, CO²⁺ or the combination thereof, optionally, the transition metal ion is Zn²⁺.

107. The composition according to claim 105, which characterized in that, the molar ratio of the compound and the transition metal ion is 1:0.4 to 1:250, optionally, the molar ratio of the compound and the transition metal ion is 1:0.4 to 1:200.

108. The composition according to claim 105, which characterized in that, the molar ratio of the compound and the transition metal ion is 1:0.4 to 1:60, optionally, the molar ratio of the compound and the transition metal ion is 1:6 to 1:16.

109. A method of preparing the compound of claim 80, which characterized in that, at least one carboxyl group of following formula III is connected to the heteroatom of a transition metal chelator moiety 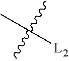
by introducing a condensation reagent under an inert atmosphere, 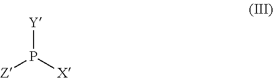
wherein X′ is 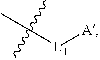
L₁ is selected from the group consisting of —CH(R₁)—, —CH(R₁)CH(R₂)—,
—CH(R₁)CH(R₂)CH(R₃)—, aryl group which is independently unsubstituted or substituted with group containing at least a coordinating atom selected from N, O and S, and heteroaryl group which is independently unsubstituted or substituted with group containing at least a coordinating atom selected from O and S;
R₁, R₂ and R₃ independently are H, C₁-C₅ alkyl group, C₁-C₅ hydroxyalkyl group, C₁-C₅ carboxy alkyl group, C₁-C₅ hydroxylamine alkyl group, C₁-C₅ N-hydroxy amide alkyl group, aryl group or heteroaryl group; or
R₂ or R₃ forms a 5-6 membered unsubstituted or substituted ring with L₂;
A′ is —COOH or —C(O)J-COOH;
J is organic group comprising amino or imino group and carbonyl group at the same time, of which the amino or imino group forms amide group with —C(O), the carboxyl group unlinked or covalently linked to L₂;
L₂ is not present or present, when L₂ is present and L₂ works as transition metal chelator motif and is —N(R₄)(R₅) or hydroxy;
R₄ and R₅ independently are hydrogen, C₀-C₅ hydroxyalkyl group, C₁-C₅ alkyl group, C₁-C₅ alkoxy group, —(CH₂)n¹(OCH₂CH₂O)n²CH(R₆)CO(R₇), unsubstituted or substituted 5-6 membered saturated heterocyclic group, unsubstituted or substituted arylalkyl group, unsubstituted or substituted aryl alkoxy group, unsubstituted or substituted aryl group, unsubstituted or substituted heteroaryl group, unsubstituted or substituted heteroaryl alkyl group, or R₄ and R₅ form a 5-6 membered unsubstituted or substituted ring, R₄ or R₅ forms a 5-6 membered unsubstituted or substituted ring with R₂ or R₃;
R₆ is hydrogen, amino, C₁-C₅ alkyl, C₁-C₅ hydroxyalkyl group, C₁-C₅ carboxy alkyl group, aryl group, unsubstituted or substituted arylalkyl group, C₁-C₅ N-hydroxy amide alkyl group, heteroaryl group or heteroaryl alkyl group;
R₇ is hydroxy, C₁-C₅ alkoxy group, —NH(CH₂CONH)n³OH;
n¹, n² and n³ independently are the number 0, 1, 2, 3, 4;
R₄ and R₅ are not hydroxy at the same time;
Y′ is same as X′;
Z′ is same as X′, or
Y′ and Z′ independently are 5-6 membered unsubstituted or substituted saturated heterocyclic group, C₁-C₅ alkyl group, C₁-C₅ hydroxyalkyl group, aryl group, C₁-C₅ carboxy alkyl group, 5-6 membered unsubstituted or substituted cycloalkyl group, or 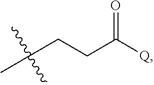
—C(O)Q is ester group, imide group or amide group.

110. A method according to claim 109, which characterized in that, the transition metal chelator moiety can be provided by 2-phenoxy-ethylamine, Phenylamine, Benzylamine, 4-Aminobenzene-1,2-diol, 5-Amino-2-hydroxybenzoic acid, Bis(pyridin-2-ylmethyl)amine, 5-Amino-8-hydroxyquinoline, Bis(pyridin-2-yl) methanamine, 4-Aminophthalic acid, tert-Butyl L-tyrosinate, DL-3-(4-Fluorophenyl)alanine, DL-4-Cyanophenylalanine, DL-4-nitro-phenylalanine, N-Benzylhydroxylamine hydrochloride, N-Phenylhydroxylamine,

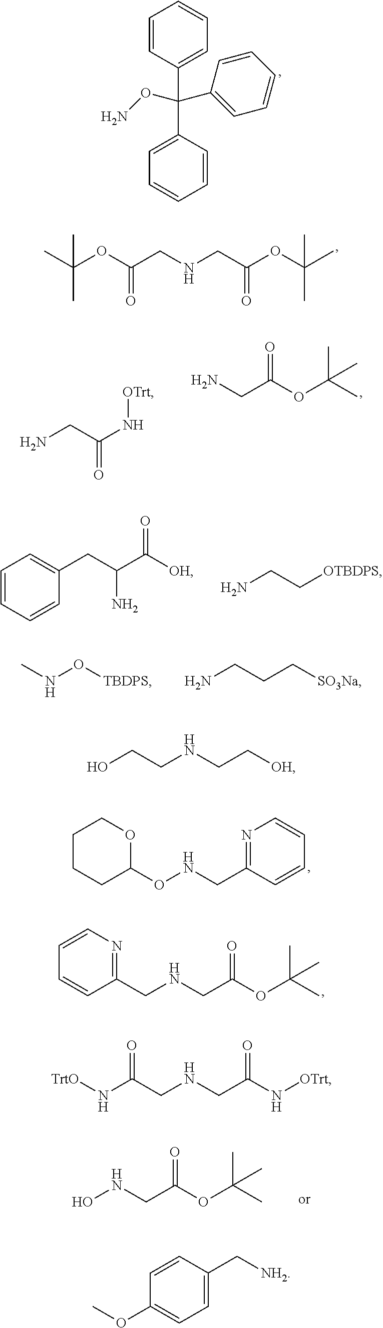

111. A method according to claim 109, which characterized in that, the structure of the formula III is

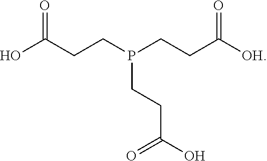 [SMILES: O=C(O)CCP(CCC(=O)O)CCC(=O)O]

112. A method of modifying an antibody, which characterized in that, the method comprises use the compound according to claim 80.

113. The method of claim 112, which characterized in that, the antibody is modified by reducing the interchain S—S bonds, optionally, the antibody is modification by selective reducing one of the interchain S—S bond.

114. The method according to claim 112, which characterized in that, modifying the antibody is in the preparation of an antibody with thiol group site-specific modifications, optionally, the antibody with thiol group site-specific modifications is an antibody drug conjugate (ADC), more optionally, the ADC is the ADC with D2, the ADC with D1, the ADC with D2+D6, the ADC with D2+D3, the ADC with D1+D6, the ADC with D1+D3, the ADC with D0+D6, the ADC with D0+D3, the ADC with D0+D4, the ADC with D2+D4, the ADC with D1+D4, the ADC with D2+D2 or the ADC with D1+D2.

115. A method of preparing the antibody with thiol group site-specific modifications, which characterized in that, the thiol group(s) is/are reduced from the interchain disulfide bonds within the antibody, and the method comprises using the compound or a salt, solvate, stereoisomer thereof according to claim 80 and transition metal ions.

116. The method according to claim 115, which characterized in that, the number of the thiol group(s) is/are 1, 2, 3, 4, 5, 6, 7 or 8.

117. The method according to claim 115, which characterized in that, the interchain disulfide bonds connect the two upper heavy chains in the hinge region, and/or the heavy chain to the light chain in the Fab region.

118. The method according to claim 115, which characterized in that, the method comprises the following steps,
(a) incubating the compound or a salt, solvate, stereoisomer thereof which works as a first reductant and the antibody in the presence of the transition metal ions in a first buffer system to selectively reduce the interchain disulfide bonds within the antibody; or
incubating the composition, wherein the compound works as the first reductant, and the antibody in the first buffer system to selectively reduce the interchain disulfide bonds within the antibody;
(b) introducing metal chelators and a modification reagent 1 to react with the reduced thiol groups resulted from step (a), wherein, the modification reagent 1 is an end capping reagent, a first linker-payload or a first thiobridge reagent, optionally, the first thiobridge reagent bears the first linker-payload or reactive groups.

119. The method according to claim 118, which characterized in that, the method further comprises the following steps,
(c) incubating the reaction product from step (b) and a second reductant in a second buffer system to reduce the interchain disulfide bonds in the reaction product, optionally, introducing the transition metal ions;
(d) introducing the incubation product from step (c) and a modification reagent 2 to react with the reduced thiol groups resulted from step (c), optionally, introducing the metal chelators, wherein, the modification reagent 2 is a second linker-payload or a second thiobridge reagent, optionally, the second thiobridge reagent bears the second linker-payload or the reactive groups.

120. The method according to claim 119, which characterized in that, the first thiobridge reagent and the second thiobridge reagent independently contain at least two substituted groups allowing a re-bridging of the thiol groups.

121. The method according to claim 119, which characterized in that, the first thiobridge reagent and the second thiobridge reagent are independently selected from the group consisting of

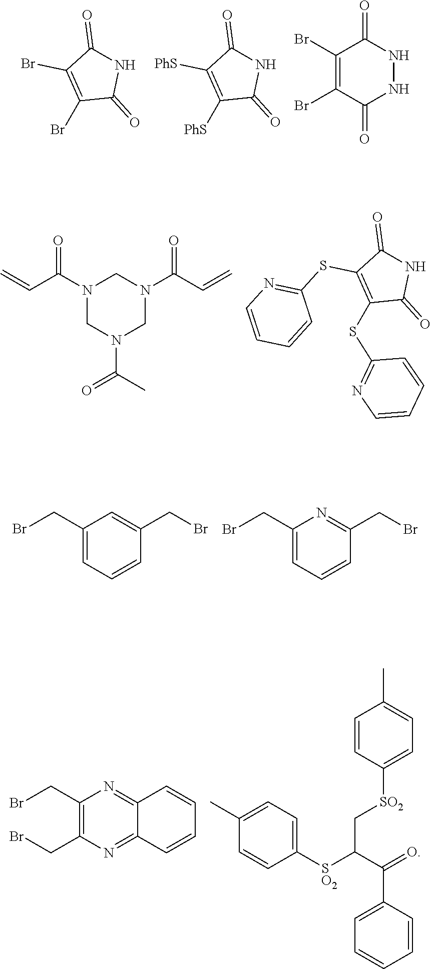

122. The method according to claim 118, which characterized in that, the reactive groups independently contain azido and/or dibenzocyclooctyne (DBCO).

123. The method according to claim 118, which characterized in that, the molar ratio of the first reductant and the transition metal ions is 1:0.4 to 1:250, optionally, the molar ratio of the first reductant and the transition metal ions is 1:0.4 to 1:60, more optionally, the molar ratio of the first reductant and the transition metal ions is 1:6 to 1:16, most optionally, the molar ratio of the first reductant and the transition metal ions is 1:12.

124. The method according to claim 118, which characterized in that, the molar ratio of the first reductant and the antibody is 3:1 to 0.5:1, optionally, the molar ratio of the first reductant and the antibody is 3:1 to 1:1, more optionally, the molar ratio of the first reductant and the antibody is 2:1 to 1:1.

125. The method according to claim 119, which characterized in that, the first buffer system and the second buffer system are independently selected from a group consisting of HEPES buffer, Histidine buffer, PBS, PB, MES buffer, BES buffer, MOPS buffer, Bis-Tris buffer, Acetate buffer, DIPSO buffer, MOPSO buffer, TES buffer, ACES buffer, MOBS buffer, TAPSO buffer, IPES buffer, ADA buffer, PIPES buffer, BTP buffer, HEPPSO buffer, POPSO buffer, EPPS buffer or Tris buffer; and/or
the concentration of the first buffer system and the second buffer system is 10 mM-100 mM; and/or
the pH value of the first buffer system and the second buffer system is 5.5 to 8, optionally, the pH value of the first buffer system and the second buffer system is 6.0 to 7.4, more optionally, the pH value of the first buffer system and the second buffer system is 6.7 to 7.4.

126. The method according to claim 118, which characterized in that, the transition metal ions are selected from a group consisting of Zn²⁺, Cd²⁺, Hg²⁺, Ni²⁺, Co²⁺ or the combination thereof, optionally, the transition metal ion is Zn²⁺.

127. The method according to claim 118, which characterized in that, the incubation temperature is 0° C. to 37° C., optionally, the incubation temperature is 0° C. to 25° C., more optionally, the incubation temperature is 0° C. to 15° C. in step (a), the incubation time is 0.2 h to 24 h in step (a);
optionally, the incubation temperature is 0° C. to 10° C. in step (a), and the incubation time is 2 h to 16 h in step (a).

128. The method according to claim 124, which characterized in that, the molar ratio of the first reductant and the antibody is 2.8:1 to 3:1, and the incubation time is 1 h to 9 h.

129. The method according to claim 119, in step (c), the molar ratio of the second reductant and the transition metal ions is 1:0.05 to 1:40, and/or the molar ratio of the second reductant and the antibody is 2.5:1 to 20:1, and/or the incubation time is 1 h to 24 h.

130. The method according to claim 119, in step (c), the molar ratio of the second reductant and the transition metal ions is 1:0.4 to 1:100, and/or the molar ratio of the second reductant and the antibody is 0.8:1 to 2.5:1, and/or the incubation time is 0.5 h to 24 h.

131. The method according to claim 119, which characterized in that, when the first thiobridge reagent bears the reactive groups, the step (b) comprises the following step,
introducing metal chelators and the first thiobridge reagent bearing the reactive groups to re-bridge the reduced thiol groups resulted from step (a), then, incubation the first linker-payload in the first buffer system to react with the reactive groups of the thiobridge group; and/or
when the second thiobridge reagent bears the reactive groups, the step (d) comprises the following step,
introducing the incubation product from step (c) and the second thiobridge reagent bearing the reactive groups to re-bridge the reduced thiol groups resulted from step (c), optionally, introducing the metal chelators, then incubating the second linker-payload in the second buffer system to react with the reactive groups of the thiobridge group.

132. The method according to claim 118, which characterized in that, the method comprises the following steps,
(a1) incubating the compound which works as the first reductant and the antibody in the presence of an effective amount of the transition metal ions in the first buffer system to selectively reduce the interchain disulfide bonds with the antibody; or
incubating the composition, in which the compound works as the first reductant, and the antibody in the first buffer system to selectively reduce the interchain disulfide bonds within the antibody;
(b1) introducing an excess amount of the metal chelators and an excess amount of the first linker-payload to react with the reduced thiol groups resulted from step (a1).

133. The method according to claim 132, which characterized in that, the antibody with thiol group site-specific modifications is the ADC with D2.

134. The method according to claim 132, which characterized in that, the method comprises the following steps,
(c2) incubating the reaction product from (b1) and the second reductant in the second buffer system to reduce the interchain disulfide bonds within the reaction product from (b1);
(d2) introducing the incubation product from step (c2) and an excess amount of the second linker-payload to react with the reduced thiol groups resulted from step (c2).

135. The method according to claim 134, which characterized in that, the antibody with thiol group site-specific modifications is the ADC with D2+D6.

136. The method according to claim 119, which characterized in that, the method further comprises the following steps:
introducing a compound which contains at least one thiol group to consume excessive said first linker-payload in step (b) and/or said second linker-payload in step (d);
purifying and recovering the resultant antibody with thiol group site-specific modifications in step (b) and/or in step (d).

137. The method according to claim 119, which characterized in that, the antibody is a monoclonal antibody, a polyclonal antibody, a mono-specific antibody or a multi-specific antibody, optionally, the antibody is a human antibody, a humanized antibody, a chimeric antibody or an antigen-binding moiety thereof, more optionally, the antibody is IgG1 or IgG4; and/or
a linker of the first linker-payload and the second linker payload is selected from any one of which the one terminal can be connected to the reduced thiol group of the antibody or the reactive groups of the thiobridge reagent, and the other terminal can be connected to the payload; and/or
the payload is selected from any one of which contains at least one substituted group allowing a connection from the payload to the linker, optionally, the payload is a cytotoxic drug, a cytokine, a nucleic acid, a radionuclide, a kinase or derivatives thereof.

138. The method according to claim 115, which characterized in that, the antibody is an engineered antibody having two amino acid substitutions of two interchain cysteines forming one interchain disulfide bond in the hinge region, optionally, the amino acid substitutions are selected from the following, cysteine to alanine, to leucine, to arginine, to lysine, to asparagines, to methionine, to aspartic acid, to phenylalanine, to praline, to glutamine, to serine, to glutamic acid, to threonine, to glycine, to tryptophan, to histidine, to tyrosine, to isoleucine or to valine, respectively, more optionally, the amino acid substitutions are selected from the following, cysteine to serine.

139. An antibody with thiol group site-specific modifications prepared by the method of claim 115.

140. The antibody with thiol group site-specific modifications according to claim 139, which characterized in that, the antibody with thiol group site-specific modifications is the ADC with D2, the ADC with D1, the ADC with D2+D6, the ADC with D2+D3, the ADC with D1+D6, the ADC with D1+D3, the ADC with D0+D6, the ADC with D0+D3, the ADC with D2+D2, the ADC with D2+D4, the ADC with D1+D2, the ADC with D1+D4 or the ADC with D0+D4.

141. A pharmaceutical composition comprising the antibody with thiol group site-modifications according to any one of claim 139 and at least a pharmaceutically acceptable carrier.

142. A method of preventing, diagnosing or treating a disease in a subject in need thereof, comprising administrating to the subject a therapeutically effective amount of the antibody with thiol group site-specific modifications according to claim 139.
